SEQUENCE LISTING (29 sequences)

SEQ ID NO: 1 - DNA, 23 bp, Unknown
  Description of Unknown TNFalpha sequence
gccgtgggtcagtatgtgagagg

SEQ ID NO: 2 - DNA, 22 bp, Unknown
  Description of Unknown TNFalpha sequence
ggagtgatcggcccccagaggg

SEQ ID NO: 3 - DNA, 23 bp, Unknown
  Description of Unknown TNFR1 sequence
ggaggagacaccatcaagagagg

SEQ ID NO: 4 - DNA, 23 bp, Unknown
  Description of Unknown TNFR1 sequence
ggggcactggatggtggcatggg

SEQ ID NO: 5 - DNA, 23 bp, Unknown
  Description of Unknown TNFR2 sequence
ggggcggggacctgggcatcagg

SEQ ID NO: 6 - DNA, 23 bp, Unknown
  Description of Unknown TNFR2 sequence
ggcccatactgcccagccggtgg

SEQ ID NO: 7 - DNA, 58 bp, Artificial Sequence
  Description of Artificial Sequence Synthetic oligonucleotide
ttaatacgactcactataggcgtgggtcagtatgtgaggttttagagctagaaatagc

SEQ ID NO: 8 - DNA, 57 bp, Artificial Sequence
  Description of Artificial Sequence Synthetic oligonucleotide
ttaatacgactcactataggagtgatcggcccccagagttttagagctagaaatagc

SEQ ID NO: 9 - DNA, 58 bp, Artificial Sequence
  Description of Artificial Sequence Synthetic oligonucleotide
ttaatacgactcactataggaggagacaccatcaagaggttttagagctagaaatagc

SEQ ID NO: 10 - DNA, 58 bp, Artificial Sequence
  Description of Artificial Sequence Synthetic oligonucleotide
ttaatacgactcactataggggcactggatggtggcatgttttagagctagaaatagc

SEQ ID NO: 11 - DNA, 58 bp, Artificial Sequence
  Description of Artificial Sequence Synthetic oligonucleotide
ttaatacgactcactataggggcggggacctgggcatcgttttagagctagaaatagc

SEQ ID NO: 12 - DNA, 58 bp, Artificial Sequence
  Description of Artificial Sequence Synthetic oligonucleotide
ttaatacgactcactataggcccatactgcccagccgggttttagagctagaaatagc

SEQ ID NO: 13 - DNA, 20 bp, Artificial Sequence
  Description of Artificial Sequence Synthetic primer
aaccgagacagaaggtgcag

SEQ ID NO: 14 - DNA, 20 bp, Artificial Sequence
  Description of Artificial Sequence Synthetic primer
tctgtgtgccagacacccta

SEQ ID NO: 15 - DNA, 21 bp, Artificial Sequence
  Description of Artificial Sequence Synthetic primer
gaagaaggggccatcactgaa

SEQ ID NO: 16 - DNA, 22 bp, Artificial Sequence
  Description of Artificial Sequence Synthetic primer
tgcaagtgagtgacaagaacag

SEQ ID NO: 17 - DNA, 20 bp, Artificial Sequence
  Description of Artificial Sequence Synthetic primer
ggtgggtcccatgtgagaga

SEQ ID NO: 18 - DNA, 20 bp, Artificial Sequence
  Description of Artificial Sequence Synthetic primer
acgagggcaagggttttgtc

SEQ ID NO: 19 - DNA, 21 bp, Artificial Sequence
  Description of Artificial Sequence Synthetic primer
gaaagcatgatccgggacgtg

SEQ ID NO: 20 - DNA, 21 bp, Artificial Sequence
  Description of Artificial Sequence Synthetic primer
gatggcagagaggaggttgac

SEQ ID NO: 21 - DNA, 21 bp, Artificial Sequence
  Description of Artificial Sequence Synthetic primer
tcaccgcttcagaaaaccacc

SEQ ID NO: 22 - DNA, 21 bp, Artificial Sequence
  Description of Artificial Sequence Synthetic primer
ggtccactgtgcaagaagaga

SEQ ID NO: 23 - DNA, 22 bp, Artificial Sequence
  Description of Artificial Sequence Synthetic primer
ttcatccacggatatttgcagg

SEQ ID NO: 24 - DNA, 20 bp, Artificial Sequence
  Description of Artificial Sequence Synthetic primer
gctggggtaagtgtactgcc

SEQ ID NO: 25 - DNA, 19 bp, Artificial Sequence
  Description of Artificial Sequence Synthetic primer
gaaggtgaaggtcggagtc

SEQ ID NO: 26 - DNA, 20 bp, Artificial Sequence
  Description of Artificial Sequence Synthetic primer
gaagatggtgatgggatttc

SEQ ID NO: 27 - protein, 467 aa, Artificial Sequence
  Description of Artificial Sequence Synthetic polypeptide
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - DNA, 18 bp, Artificial Sequence
  Description of Artificial Sequence Synthetic oligonucleotide
ttaatacgactcactata

SEQ ID NO: 29 - DNA, 20 bp, Artificial Sequence
  Description of Artificial Sequence Synthetic oligonucleotide
gttttagagctagaaatagc

(19) United States
(12) Patent Application Publication
(10) Pub. No.: US 2023/0103554 A1
(43) Pub. Date: Apr. 6, 2023

(54) TNFa SIGNALING TRIGGERS TUMOR-PROMOTING INFLAMMATION THAT CAN BE TARGETED TO THERAPY

(71) Applicant: Baylor College of Medicine, Houston, TX (US)

(72) Inventors: Leonid S. Metelitsa, Houston, TX (US); Julie Tomolonis, Houston, TX (US)

(21) Appl. No.: 17/907,312

(22) PCT Filed: Apr. 1, 2021
(86) PCT No.: PCT/US21/25398

§ 371 Date: Sep. 26, 2022

Related U.S. Application Data
(60) Provisional application No. 63/005,019, filed on Apr. 3, 2020.
(60) Provisional application No. 63/019,289, filed on May 2, 2020.

(51) Int. Cl. A61K 38/17; A61P 35/00; A61K 45/06; A61K 39/395; A61P 29/00

(52) U.S. Cl. CPC A61K 38/1793; A61P 35/00; A61K 45/06; A61K 39/3955; A61P 29/00; A61K 2039/545

(57) ABSTRACT

TNFR2-expressing neuroblastoma cells activate monocytes via contact-dependent mTNFα signaling, leading to production of sTNFα and IL-6 by monocytes that in turn bind to TNFR1 and IL-6R on neuroblastoma cells and stimulate tumor growth via activation of NF-κB and Sta3 signaling pathways. Growing tumor recruit additional monocytes resulting in a self-promoting inflammation that fuel tumor growth. TNF inhibitors can terminate this feed-forward signal amplification loop and inhibit tumor growth. Embodiments of the disclosure include a method of using TNF inhibitors for the treatment of neuroblastoma in a subject in need thereof. Also disclosed herein is a method of a method of using TNF inhibitors for reducing tumor-promoting inflammation in an individual with cancer.

This application claims priority to U.S. Provisional Patent Application Ser. No. 63/005019, filed Apr. 3, 2020, and also to U.S. Provisional Patent Application Ser. No. 63/019,289, filed May 2, 2020, both of which applications are incorporated by reference herein in their entirety.

### STATEMENT REGARDING FEDERALLY SPONSORED RESEARCH OR DEVELOPMENT

This invention was made with government support under CA116548 awarded by the National Institutes of Health. The government has certain rights in the invention.

### TECHNICAL FIELD

Embodiments of the disclosure concern at least the fields of cellular biology, molecular biology, physiology, and medicine, including cancer medicine.

### BACKGROUND

Neuroblastoma (NB) is a poorly differentiated, aggressive pediatric solid tumor for which half of high-risk cases have no identified genetic alteration. While such tumors are often infiltrated by M2-like macrophages that generate an inflammatory gene signature predictive of poor outcome, the initiation mechanism of this pro-tumorigenic inflammation remains undefined. As such, there is a need in the art for methods of inhibiting pro-tumorigenic inflammation associated with cancers, including neuroblastoma. The present disclosure provides solutions to long-felt needs in the art for treatment of neuroblastoma.

Neuroblastoma (NB) is a heterogeneous pediatric tumor of neural crest origin. It is the second most common solid tumor in children and causes 15% of all pediatric cancer deaths (1). Treatment remains a significant clinical challenge due to the range of courses the disease may follow, from spontaneous regression to treatment-resistant progression and death (2). High-risk NB represents about half of total diagnoses; these cases present with aggressive, unfavorable histology, are typically metastatic, and are difficult to treat (3). Despite intensive therapy regimens including surgery, radiation, high-dose chemotherapy with stem cell transplantation, retinoic acid, and antibody-based immunotherapy, the long-term survival for patients with high-risk disease is less than 50% (4).

Approximately half of high-risk NB tumors are characterized by amplification of the MYCN and/or ALK oncogene(s) (5). These genetic aberrations activate transcription of genes involved in cell cycle progression and repress those that induce cell differentiation leading to highly proliferative, dedifferentiated tumors (6, 7). However, the remaining half of high-risk NB tumors lack clearly defined genetic drivers of tumor progression. High-risk MYCN-non-amplified NB tumors are often infiltrated by immune cells including M2-like tumor-associated macrophages (TAMs) (8, 9). Moreover, patients with tumors expressing higher levels of an inflammatory gene signature associated with monocytes/TAMs (CD14, CD16, IL6, IL6R, and TGFB1) had worse five-year progression-free survival than patients without this signature, highlighting the role of TAMs in promoting a tumor microenvironment (TME) favorable for NB growth (9, 10).

TAMs are a major component of the TME in many types of solid tumors. They originate from tissue-resident macrophages and/or bone marrow-derived monocytes that extravasate and differentiate within the tumor parenchyma. In most tumor types, including NB, TAMs promote growth and metastasis and inhibit antitumor immunity (11, 12). Therefore, targeting TAMs may be an attractive strategy for cancer therapy (13). However, early-stage clinical testing of reagents that target TAMs, such as colony stimulating factor 1 receptor (CSF1R) inhibitors and monoclonal antibodies, have produced modest therapeutic activity in cancer patients as single agents (14-16).

To effectively neutralize TAMs and/or their tumor-promoting function, it is critical to understand the mechanisms by which they interact with tumor cells. Previous studies demonstrated that NB cells stimulate monocytes to produce IL-6, which was shown to be partially responsible for the promotion of NB growth in mice (9). However, the mechanism by which NB cells interact with monocytes/macrophages and initiate this tumor-promoting inflammatory response remains unknown.

Certain embodiments of the disclosure describe a novel inflammatory feedback loop between NB cells and monocytes/macrophages that is triggered and sustained by TNFα signaling. Certain embodiments demonstrate that NB cells activate monocytes through a contact-dependent reverse signaling interaction between TNFR2 on the NB cell surface and membrane-bound TNFα (mTNFα) on monocytes. This interaction may initiate downstream NF-κB signaling in monocytes, leading to production of pro-tumorigenic cytokines including IL-6, G-CSF, IL-1, and soluble (s)TNFα. These cytokines then complete the feedback loop by stimulating NB cell proliferation that results in enhanced tumor growth and angiogenesis. In certain embodiments, this pro-inflammatory signaling loop is completely abrogated by FDA-approved etanercept, a TNFα-neutralizing Fc-TNFR2 fusion protein, leading to reversal of monocyte/macrophage-mediated NB growth promotion in vitro and in vivo.

### BRIEF SUMMARY

The present disclosure is directed to compositions, kits, and methods for treating cancer, including neuroblastoma, and methods for reducing tumor-promoting inflammation, including tumor-promoting inflammation associated with neuroblastoma. Certain methods encompassed herein comprise the use of one or more tumor necrosis factor (TNF) inhibitors. In some embodiments, one or more TNF inhibitors are used to treat neuroblastoma. In some embodiments, one or more TNF inhibitors are used to reduce tumor-promoting inflammation.

The TNF inhibitor(s) used in any of the methods encompassed herein may comprise any TNF inhibitor known in the art. The TNF inhibitor(s) may directly or indirectly inhibit, bind, block, and/or neutralize TNFα (including soluble and/or membrane-bound TNFα), TNFR1, TNFR2, or a combination thereof, in any manner. The TNF inhibitor(s) may comprise at least one small molecule, immunotherapy, cell therapy, peptide, peptide derivative, antibody, fusion protein, glycoprotein, nucleic acid, nucleic acid derivative, or a combination thereof. The TNF inhibitor(s) may comprise etanercept, infliximab, certolizumab, golimumab, adalimumab, or a combination thereof.

Certain embodiments encompassed herein concern methods for reducing tumor-promoting inflammation in an individual. In some embodiments, reducing tumor-promoting inflammation inhibits cancer growth, reduces tumor size, reduces tumor aggressiveness, reduces angiogenesis, sensitizes tumors to chemotherapy, increases the efficacy of effector lymphocytes against tumors, or a combination thereof. Tumor-promoting inflammation may comprise an increase in the production and/or expression of IL-6 and/or TNFα (including soluble and/or membrane-bound TNFα), and other pro-promoting cytokines such as IL-1, IL-4, IL-8, IL-10, IL-13, IL-17, IL-33, etc. Tumor-promoting inflammation can be in part mediated via activation of NF-kB signaling pathway which activity is inversely correlated with the level of IkBa protein expression. The increase or decrease of IL-6, TNFα, and/or IκBα may be relative to a baseline established in an individual, a concentration found in a non-tumorous location in the individual, a normal concentration found in an individual who does not have cancer, or other normal concentration known in the art.

Certain embodiments encompassed herein concern contacting cells and/or extracellular fluid, in or from an individual, with one or more TNF inhibitors. The cells contacted may be any cell type, including any cell capable of producing IL-6 and/or TNFα, or expressing a TNF receptor, such as monocytes and/or macrophages. The cells contacted may be cancerous, including any cancer cell expressing one or more TNF and/or IL-6 receptors, such as neuroblastoma cells. The cells may be contacted in vitro, in vivo, and/or ex vivo. The extracellular fluid may comprise any extracellular fluid from an individual, such as blood, plasma, serum, interstitial fluid, tumor interstitial fluid, or a combination thereof. The extracellular fluid may comprise cell culture fluid, including media used to culture cells.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawing, in which:

FIGS. 1A-1C show that neuroblastoma (NB) induces monocyte IL-6 production in a contact and NF-κB dependent mechanism. (FIG. 1A) Representative panel of NB lines and freshly isolated monocytes were directly co-cultured in 4:1 (NB:mono) ratio for 24 hours. Co-culture supernatants were analyzed for IL-6 levels by ELISA. (FIG. 1B) Two different monocyte donors were cultured in control medium, 50% 7-day NB conditioned medium (0.22 μm sterile filtered), or directly with NB in 4:1 ratio for 24 hours and cytokine levels determined by IL-6 ELISA. From left to right the bars are 50% Control, 50% Condition, and NB Co-culture. (FIG. 1C) NB monocyte co-cultures were treated with IKK inhibitor VII, CAS 873225-46-8, (125 nM) or VEH Control (0.1% DMSO) for 24 hours and IL-6 levels measured by IL-6 ELISA. **** p<0.0001;

FIGS. 2A-2C show that NB expresses functional TNF receptors (TNFRs). (FIG. 2A) Representative panel of NB cell lines were analyzed for TNFR1 and TNFR2 surface expression by flow cytometry. (FIG. 2B) NB cell lines were treated with recombinant human TNFα (rhTNFα) (10 ng/mL) for 12 hours and intracellular IκBα levels were determined by PhosFlow flow cytometry and normalized to their respective untreated controls (set to 1). (FIG. 2C) NB cell lines were cultured with human monocytes for 24 hours and intracellular flow cytometry intracellular IκBα levels were determined by PhosFlow flow cytometry and normalized to their respective NB only controls (set to 1). Surface staining of CD45 and GD2 were used to differentiate Monocyte (CD45+, GD2−) and NB (CD45−, GD2Low-high) populations. * p<0.05, ** p<0.01;

FIGS. 3A-3C show that TNFR1 and TNFR2 can be successfully knocked out using CRISPR/Cas9 gene editing. (FIG. 3A) Schematic showing TNFRSF1A (TNFR1) and TNFRSF1B (TNFR2) exons targeted for deletion, designed to create frameshift mutations and disrupt protein synthesis. (FIG. 3B) NB cell lines were electroporated with sgRNA/Cas9 RNPs and PCR on genomic DNA was used to verify successful editing. Shown, PCR across deletion regions compared to WT and validated homozygous and heterozygous positive controls, respectively. Band densitometry in ImageJ was used to estimate approximate editing efficiency. (FIG. 3C) Single cell clones were isolated from edited cell lines and homozygous knockout confirmed by PCR, sequencing and loss of surface expression by flow cytometry (shown);

FIGS. 4A-4B show NB TNFR2 is critical for monocyte activation. (FIG. 4A) TNFR1 KO, TNFR2 KO, and WT SK-NBE2 cell lines were cultured with human monocytes for 24 hours. Intracellular expression of IκBα was evaluated within the GD2+,CD45− NB population. (FIG. 4B) Monocyte activation was assessed by measuring IL-6 concentration by ELISA from co-culture supernatants. ** p<0.01;

FIGS. 5A-5C demonstrate that monocyte mTNFa is critical for monocyte activation. (FIG. 5A) NB and monocyte co-cultures were treated with TNF converting enzyme inhibitor TAPI (40 μM) or Vehicle control (DMSO, 0.2%) for 24 hours. Surface expression of mTFNa within CD45+, GD2− monocyte population was performed using flow cytometry. TAPI is shifted right compared to Vehicle and Isotype. (FIG. 5B) TNFa and IL-6 levels within co-culture supernatants were determined by ELISA. (FIG. 5C) Intracellular IκBα expression level within CD45+, GD2− monocyte population was determined by PhosFlow flow cytometry. Mono Alone is shifted right compared to +NB+Vehicle (VEH) and compared to +NB+TAPI and compared to Isotype. ** p<0.01, **** p<0.0001;

FIGS. 6A-6C show that research-grade TNF neutralizing antibodies reduce tumor-promoting inflammation. A panel of 5 NB cell lines were co-cultured with freshly isolated monocytes in the presence of TNF, TNFR1, TNFR2, or Mouse IgG1 isotype control antibodies for 24 hours. (FIG. 6A) IκBα expression level in NB population (CD45−, CD56+) was determined by intracellular PhosFlow. The IκBα MFI values were normalized to the isotype control (dashed line) and the results from all 5 cell lines averaged. (FIG. 6B) Normalized IκBα expression, as described previously, in CD45+, CD56 monocyte population. (FIG. 6C) Co-culture supernatants were analyzed for IL-6 levels by ELISA. Cytokine production was normalized to the isotype treated condition to evaluate magnitude of reduction and pooled across the 5 cell lines. * p<0.05, ** p<0.01, *** p<0.001, **** p<0.0001; the bar graph reading from left to right corresponds to the legend from top to bottom;

FIGS. 7A-7C show that etanercept disrupts tumor promoting inflammation, in vitro. SK-N-AS cell line was co-cultured with freshly isolated human monocytes and treated with 10 μm/mL etanercept or 10 μm/mL IgG vehicle control for 24 hours. (FIG. 7A) The concentration of TNFa and IL-6 in the co-culture supernatants was determined by ELISA. (FIG. 7B) Intracellular IκBα levels within CD14− GD2+ NB (SK-N-AS) population was determined by PhosFlow flow cytometry. IκBα MFI was normalized to NB alone to show the magnitude of drug effect. (FIG. 7C) Intracellular IκBα levels within CD14+ GD2− freshly isolated human monocyte populations were determined by PhosFlow flow cytometry. IκBα MFI was normalized to NB alone to show the magnitude of drug effect. * p<0.05, ** p<0.01, *** p<0.001, **** p<0.0001;

FIGS. 8A-8B demonstrate that etanercept reduces NB growth in vitro. (FIG. 8A) Luciferase expressing CHLA-255-luc and SK-NAS-luc were cultured in 1-16,000 pg/mL rhTNF for 48 hours and viability of the cells was measured by luminescence. Data was normalized to untreated control and graphed as % Viability versus Log10 of TNF concentration. (FIG. 8B) CHLA-255-luc and SK-N-AS-luc were cultured with freshly isolated human monocytes treated with 10 μg/mL etanercept or 10 μg/mL IgG vehicle control for 72 hours. Luminescence of the NB was used to evaluate relative viability of the cell line following treatment with etanercept. * p<0.05, *** p<0.001, **** p<0.0001;

FIGS. 9A-9D show that etanercept treatment eliminates monocyte-induced tumor growth in vivo. Luciferase-labeled NB and freshly isolated human monocytes were embedded in Matrigel and injected into the subcutaneous right flank of 8-week female NSG mice. Tumors were treated biweekly with 100 μg etanercept or control IgG in a vehicle control. (FIG. 9A) Tumor growth was tracked indirectly through luminescence imaging using IVIS Imaging system. (FIG. 9B) Average tumor luminescence of each group over time. After 3 weeks, mice were sacrificed and the tumor size was analyzed directly by weight. The bottom line is NB+Mono+FcTNFR and the top line is NB+Mono+IgG. (FIG. 9C) Representative image of 5 tumors from each group. (FIG. 9D) Tumor weight by group after Log10 transformation of weight (in mg). * p<0.05, ** p<0.01;

FIG. 10 shows that etanercept eliminates and/or disrupts tumor-promoting inflammation, allowing for effective immunotherapy of neuroblastoma. TNFR2 expression by neuroblastoma reverse signals through mTNFa on monocytes leading to downstream activation of NF-κB. Activation of NF-κB in monocytes leads to production of tumor-promoting cytokines like IL-6 which activates downstream STAT3 in NB, as well as sTNF which activates NF-κB in NB through TNFR1. Together these functions promote increased NB survival and proliferation. Etanercept effectively neutralizes both TNF isoforms, blocking monocyte NF-κB activation and IL-6 production, reducing tumor growth;

FIGS. 11A-11C demonstrate that etanercept treatment eliminates monocyte-induced tumor growth in vivo. Luciferase-labeled NB and freshly isolated human monocytes were embedded in Matrigel and injected into the subcutaneous right flank of 8-week female NSG mice. Tumors were treated bi-weekly with 100 μg etanercept or control IgG in a vehicle control. (FIG. 11A) Tumor growth was tracked indirectly through luminescence imaging using IVIS Imaging system. (FIG. 11B) Average tumor luminescence of each group over time. NB+Mon.+FcTNFR is the bottom line, and NB+Mon+IgG is the top line. (FIG. 11C) Mice were sacrificed at day 25 and their tumors weights measured directly. *** p<0.001;

FIGS. 12A-12F demonstrate NB induces monocyte IL-6 production in contact-and NF-κB-dependent mechanism. (FIG. 12A) Representative panel of NB lines and freshly isolated monocytes were directly co-cultured at 4:1 (NB:mono) ratio for 24 hours. Co-culture supernatants were analyzed for IL-6 levels by ELISA. Mean±SD, representative of two independent experiments run in duplicate. A=MYCN amplified, NA=MYCN non-amplified. (FIG. 12B) Two monocyte donors were cultured in control medium (CTRL), 50% seven-day CHLA-255 conditioned medium (CM), or directly with CHLA-255 at a 4:1 ratio (+NB) for 24 hours and cytokine levels determined by IL-6 ELISA. Mean±SD, representative of two independent experiments run in duplicate. From left to right, the bars are CTRL, CM, and +NB. (FIG. 12C) CHLA-255/monocyte co-cultures were treated with IKK inhibitor VII (IKKi, 125 nM) or vehicle control (Veh., 0.1% DMSO) for 24 hours and IL-6 levels measured by IL-6 ELISA. Mean±SD, representative of two independent experiments run in duplicate. (FIG. 12D) TNFα levels in co-culture shown in (FIG. 12C) were measured by TNFα ELISA. (FIG. 12E) Five NB cell lines were co-cultured with monocytes treated with 10 μg/mL anti-TNFα antibody or isotype control for 24 hours. IL-6 levels were measured by ELISA and normalized to isotype treated control. Mean normalized IL-6 secretion across five cell lines±SD, representative from two experiments run in duplicate. (FIG. 12F) Intracellular IκBα expression level within CD45+GD2− monocyte population was determined by PhosFlow flow cytometry and IκBα MFI normalized to isotype treated control. Results are averaged across all five cell lines±SD, representative from two experiments run in duplicate. * p<0.05, **** p<0.0001;

FIGS. 13A-13F demonstrate monocyte mTNFα is sufficient for monocyte activation. (FIG. 13A) CHLA-255 and monocytes were cultured alone in the presence of LPS (100 ng/mL) and GolgiStop (1.5 μL/mL) for six hours and intracellular accumulation of TNFα was measured by flow cytometry. Data are from a representative of two experiments run in duplicate. (FIG. 13B) WT and TNFα KO CHLA-255 NB was co-cultured with monocytes for 24 hours and supernatant IL-6 levels were measured by IL-6 ELISA. Mean±SD of six validated KO clones. (FIG. 13C) NB and monocyte co-cultures were treated with TNF converting enzyme inhibitor TAPI (40 μM) or vehicle control (Veh., 0.2% DMSO) for 24 hours. Surface expression of mTNFα within CD45+GD2− monocyte population was performed using flow cytometry. MFI as indicated. Data are from a representative of two experiments run in duplicate. The TAPI peak is shifted right compared to Vehicle and Isotype. (FIG. 13D) TNFα and (FIG. 13E) IL-6 levels within co-culture supernatants were determined by ELISA. Mean fold change in cytokine production±SD from three independent experiments run in duplicate. (FIG. 13F) Intracellular IκBα expression level within CD45+GD2− monocyte population was determined by PhosFlow flow cytometry. Data are representative of two independent experiments. ** p<0.01, **** p<0.0001;

FIGS. 14A-14E demonstrate NB TNFR2 expression is required for monocyte activation. (FIG. 14A) Representative panel of NB cell lines were analyzed for TNFR1 and TNFR2 surface expression (black line) by flow cytometry. Results are shown as percentage TNFR positive with gating threshold from isotype (grey histogram). Data are representative of two experiments run in duplicate. (FIG. 14B) TNFRSF1A mRNA (TNFR1) expression was evaluated by qRT-PCR. C(t) values for TNFRSF1A were normalized against GAPDH housekeeping gene. Mean±SD run in duplicate. (FIG. 14C) TNFRSF1B mRNA (TNFR2) expression was evaluated by qRT-PCR. C(t) values for TNFRSF1B were normalized against GAPDH housekeeping gene. Mean±SD run in duplicate. (FIG. 14D) WT, TNFR1, and TNFR2 KO SK-N-BE(2) cells were co-cultured with human monocytes from two different donors for 24 hours. Supernatant IL-6 levels were determined by ELISA. Mean IL-6 production normalized from monocyte background to WT co-culture±SD, normalized data from two monocyte donors run in duplicate. (FIG. 14E) WT, TNFR1, and TNFR2 SK-N-BE(2) KO clones were co-cultured with human monocytes from two different donors for 24 hours. Intracellular IκBα expression level within CD45-GD2+NB population was determined by PhosFlow flow cytometry and IκBα MFI normalized to NB alone control. Mean normalized IκBα MFI±SD, normalized data from two monocyte donors run in duplicate. ** p<0.01, **** p<0.0001;

FIGS. 15A-15G demonstrate Etanercept reduces pro-tumorigenic signaling in vitro. (FIGS. 15A-E) CHLA-255 was cultured with freshly isolated human monocytes for 24 hours in the presence of 10 μg/mL etanercept (Etan) or IgG in a vehicle control (Ctrl). (15A) TNFα level within co-culture supernatant was determined by TNFα ELISA. Mean TNFα concentration±SD of representative data from two monocyte donors run in triplicate. (FIG. 15B) Supernatant IL-6 levels were determined by ELISA. Mean IL-6 concentration±SD of representative data from two monocyte donors run in triplicate (FIG. 15C) Intracellular IκBα expression level within CD14+GD2− monocyte population was determined by flow cytometry and IκBα MFI was normalized to monocyte only control. Mean normalized IκBα MFI±SD and representative from experiments with two monocyte donors run in triplicate. (FIG. 15D) Intracellular IκBα expression level within CD14-GD2+NB population was determined by flow cytometry and IκBα MFI was normalized to NB only control. Mean normalized IκBαMFI±SD, representative from experiments with two monocyte donors run in triplicate. (FIG. 15E) Multiplex Luminex cytokine assay was performed on co-culture supernatants. Heatmap shows mean log10 transformation of cytokine concentration in pg/mL. Red arrows indicate cytokines significantly upregulated during co-culture and ablated by etanercept treatment across two monocyte donors. Luminex assay performed in duplicate on conditions run in triplicate. P values for relevant comparisons shown in FIG. 29. (FIG. 15F) CHLA-255-luc was cultured with freshly isolated human monocytes treated with 10 μg/mL Etan or Ctrl for 72 hours. NB luminescence was used to evaluate relative viability of cell line. Results are normalized mean luminescence±SD, n=6 replicates per condition. (FIG. 15G) CHLA-255 was pulsed for 10 minutes with CellTrace Violet (CTV) then co-cultured with monocytes in the presence of 10 μg/mL Etan or Ctrl for four days. CTV staining was used to determine NB cell divisions. Cells undergoing multiple (2+) divisions were quantified as a percentage of total cells and normalized to NB (0) and Ctrl (1). Shown is mean normalized percentage of cells undergoing multiple divisions±SD of representative data from two monocyte donors run in triplicate. * p<0.05, ** p<0.01, *** p<0.001, **** p<0.0001;

FIGS. 16A-16E demonstrate etanercept treatment inhibits tumor growth in NB/monocyte xenogeneic mouse model. (FIG. 16A) In vivo subcutaneous xenograft model. CHLA-255-luc and freshly isolated human monocytes were embedded in Matrigel and injected into the subcutaneous right flank of eight-week female NSG mice. Mice were injected i.p. bi-weekly with 5 mg/kg (100 μg) etanercept (Etan) or IgG in a vehicle control (Ctrl), and tumor growth was tracked indirectly through luminescence imaging using IVIS imaging system. (FIG. 16B) Luminescence readings for each mouse (n=10/group) measured in radiance (photons/second/cm2/steradian). (FIG. 16C) Graphical representation of total luminescence per tumor from (FIG. 16B) over time. (FIG. 16D) Mean tumor luminescence over time, two-way ANOVA with Bonferroni multiple comparisons test (n=10 per group). The top line is NB/Mon+Ctrl. (FIG. 16E) Representative tumors for each group following sacrifice at day 21. ** p<0.01;

FIGS. 17A-17F demonstrate etanercept treatment alters NB tumor microenvironment. (FIG. 17A) Tumors recovered from mice in FIG. 16 were snap frozen in OTC media, thin sectioned, and stained for murine CD31 expression (red) and DAPI counterstain (blue). Results shown are representative images from five fields of view (FOVs) per tumor, with five tumors per group. Scale bar 50 μm. (FIG. 17B) Total number of CD31+ regions per FOV with box and whisker plot showing quartiles±range (Ctrl n=15, Etan n=25). (FIG. 17C) Size of each CD31+ area quantified per FOV; shown is median CD31+ area per FOV with box and whisker plot showing quartiles±range (Ctrl n=15, Etan n=25) (FIG. 17D) Longest distance across each CD31+ area (Max Feret distance) per FOV; shown is median distance per FOV with box and whisker plot showing quartiles±range (Ctrl n=15, Etan n=25). (FIG. 17E) Eukaryotic mRNA-seq was performed on tumor samples, and mouse transcripts were excluded from analysis by XenoFilterR program. Differentially regulated genes were identified using two-fold expression difference and adjusted P value<0.05 as cutoffs. (FIG. 17F) Gene-set enrichment analysis (GSEA) revealed pathways downregulated by Etan treatment and categorized based on function (pathways shown with ≥5 genes differentially regulated and P value<0.01);

FIGS. 18A-18C demonstrate MYCN-non-amplified NB tumors are enriched in TNFα signaling pathways. (FIG. 18A) Kocak Neuroblastoma dataset of stage IV tumors only (n=211) was subjected to differential gene expression analysis, based on the presence or absence of MYCN amplification (A=amplified, NA=Non-amplified). Volcano plots depicting differential gene expression are overlayed with genes from the indicated pathways in red. Genes that are differentially expressed in NA tumors are left of the dashed axis line, and P values for pathway enrichment are indicated. A: n=65, NA: n=146. (FIG. 18B) Seeger MYCN-non-amplified dataset was subjected to Kaplan-Meier survival analysis based on gene expression using p-scan method set to a minimum of 10 samples per group. Shown are relapse free survival curves for TNFα (TNF; where high is the top line), TNFR1 (TNFRSF1A; where low is the top line), and TNFR2 (TNFRSF1B; where low is the top line). (FIG. 18C) Seeger MYCN-non-amplified Neuroblastoma dataset was subjected to differential gene analysis between patients that relapsed (*) or had no progression. Unsupervised clustering of KEGG “TNF_Signaling_Pathway” is displayed as a heatmap of differentially expressed genes; blue boxes indicate clusters of patients with distinct gene expression patterns that correlate highly with progression. Overall pathway association with progression free survival P=8.0×10⁻³;

FIGS. 19A-19D demonstrate IL-6 and TNFα cytokine production is regulated by canonical NF-κB signaling. (FIG. 19A) CHLA-255 was co-cultured with human monocytes in the presence of IKK inhibitor VII (IKKi) at concentrations ranging from 0-1000 nM and IL-6 concentration was determined by IL-6 ELISA. Mean IL-6 level±SD run in duplicate. (FIG. 19B) Normalized IL-6 level plotted against log10 IKKi (nM); non-linear regression (multiple slopes) was fitted to data to estimate the IC50 of IL-6 release. IC50 and R2 for curve goodness-of-fit as indicated. (FIG. 19C) CHLA-255 was co-cultured with human monocytes in the presence of IKK Inhibitor VII (IKKi) at concentrations ranging from 0-1000 nM and TNFα level was determined by TNFα ELISA. Mean TNF level±SD run in triplicate. (FIG. 19D) Normalized TNFα level plotted against log10 IKKi (nM); non-linear regression (multiple slopes) was fitted to data to estimate the IC50 of TNFα release. IC50 and R2 for curve goodness-of-fit as indicated;

FIGS. 20A-20G demonstrate generation and validation of TNF KO clones. (FIG. 20A) TNF mRNA expression was evaluated by qRT-PCR. C(t) values for TNF cDNA were normalized against GAPDH housekeeping gene then CHLA-255 cell line. Mean±SD run in duplicate. (FIG. 20B) Gel electrophoresis of qRT-PCR products from FIG. 20A) where TNF cDNA corresponds to 510 bp product as in positive U937 control (non-specific bands in NB lines likely from gDNA contamination). Samples were run on same gel, but in non-contiguous lanes. (FIG. 20C) KO strategy for TNF, targeting the first exon with predicted loss-of-function mutation. (FIG. 20D) PCR showing successful editing of TNF in U937; WT locus 954 bp, KO 727 bp. Log2 molecular weight ladder shown. (FIG. 20E) TNFα ELISA of U937 lysates, bulk CRISPR-edited population, and two representative KO clones. All samples were stimulated with PMA (20 ng/mL) and ionomycin (1 μg/mL) to induce TNFα production. (20F) PCR showing successful editing of TNF in CHLA-255 NB; WT locus 954 bp, KO 727 bp. Log2 molecular weight ladder shown. (FIG. 20G) Sanger sequencing for three representative KO clones aligned to WT relative to upstream and downstream sgRNA sites;

FIGS. 21A-21C demonstrate generation of TNFR1 and TNFR2 KOs in NB. (FIG. 21A) KO strategy for TNFRSF1A (TNFR1) and TNFRSF1B (TNFR2). Both deletions occur in N-terminal extracellular ligand binding domain and are predicted non-sense mutations. (FIG. 21B) PCR showing successful editing of TNFRSF1A and TNFRSF1B loci in unsorted, bulk SK-N-AS cell population; WT TNFRSF1A locus 2349 bp, KO 1085 bp. WT TNFRSF1B locus 1547 bp, KO 1086 bp. Log2 molecular ladder shown. Samples run on same gel, but in non-contiguous lanes. (FIG. 21C) Single clones of TNFR1 and TNFR2 KOs were analyzed for receptor surface expression by flow cytometry (blue line) compared to WT (red histogram) and appropriate negative control (grey histogram). Representative data from four validated KO clones in SK-N-BE(2);

FIGS. 22A-22E demonstrate TNFR KOs induce senescence and/or differentiation in NB. (FIG. 22A) Representative SK-N-AS WT, TNFR1, and TNFR2 KOs at six weeks post-CRISPR editing were imaged at 20× magnification using Nikon DS-Fi1/Elements Software. Scale bar 20 μm. (FIG. 22B) Western blot of neuron-specific enolase (enolase-2, NSE) and housekeeping protein GAPDH in WT, TNFR1 KO, and TNFR2 KO SK-N-BE(2) cells. Samples were run on same gel, but in non-contiguous wells. (FIG. 22C) Quantification of NSE expression relative to GAPDH expression using gel analyzer in ImageJ/FIJI and normalized to WT SK-N-BE(2). Mean normalized NSE±SD from two replicates. (FIG. 22D) Western blot of growth associated protein 43 (GAP43) and housekeeping protein GAPDH in WT, TNFR1 KO, and TNFR2 KO SK-N-BE(2) cells. Samples were run on same gel, but in non-contiguous wells. (FIG. 22E) Quantification of GAP43 expression relative to GAPDH expression using gel analyzer in ImageJ/FIJI and normalized to WT SK-N-BE(2). Mean normalized GAP43±SD from two replicates. * p<0.05;

FIGS. 23A-23C demonstrate NB NF-κB is activated by sTNFα. (FIG. 23A) NB cell lines were co-cultured with monocytes for 24 hours. Intracellular IκBα expression level within CD45-GD2+NB population was determined by PhosFlow flow cytometry and IκBα MFI normalized to NB alone control. Mean normalized IκBα MFI±SD from cultures run in duplicate. (FIG. 23B) NB cell lines were cultured with 5 ng/mL TNFα, IL-6, IL-6+sIL-6Rα, or a combination of all three for 12 hours. Intracellular IκBα expression level was determined by PhosFlow flow cytometry and IκBα MFI normalized to NB untreated (PBS) control. Mean normalized IκBα MFI±SD from cultures run in duplicate. (FIG. 23C) NB cell lines were co-cultured with monocytes treated with TAPI (40 μM) or DMSO vehicle control (Veh.)(0.2%) for 24 hours. Intracellular IκBα expression level within CD45-GD2+NB population was determined by PhosFlow flow cytometry and IκBα MFI normalized to NB alone control. Mean normalized IκBα MFI±SD from co-cultures run in duplicate. * p<0.05, ** p<0.01;

FIGS. 24A-24C demonstrate research-grade TNF neutralizing antibodies reduce tumor-promoting inflammation. Five NB cell lines were co-cultured with freshly isolated monocytes in the presence of TNF, TNFR1, TNFR2, or mouse IgG1 isotype control antibodies for 24 hours. (FIG. 24A) Intracellular IκBα expression level within CD45-GD2+ NB population was determined by PhosFlow flow cytometry and IκBα MFI normalized to isotype control. Shown is mean normalized IκBα and average normalized response across all cell lines±SD (right). (FIG. 24B) Intracellular IκBα expression level within CD45+GD2− monocyte population was determined by PhosFlow flow cytometry and IκBα MFI normalized to isotype control. Shown is mean normalized IκBα and average normalized response across all cell lines±SD (right). (FIG. 24C) Co-culture supernatants were analyzed for IL-6 levels by ELISA. Data shown is mean IL-6 and average normalized IL-6 levels across all cell lines±SD (right). In all images of FIG. 24, the bars from left to right in the graphs coincide with the key reading from top to bottom. * p<0.05, ** p<0.01, *** p<0.001, **** p<0.0001;

FIGS. 25A-25B demonstrate TNFα signaling regulates pro-tumorigenic inflammation. (FIG. 25A) Luminex panel run on supernatants from SK-N-AS and monocyte co-cultures. (FIG. 25B) Luminex panel run on supernatants from SK-N-BE(2) and monocyte co-cultures. Heatmaps represent mean cytokine level from n=3 biological replicates per condition shown as log10 of the concentration in pg/mL. Etan=etanercept, Ctrl=IgG control;

FIGS. 26A-26B demonstrate Etanercept blocks interaction of NB and monocytes. (FIG. 26A) SK-N-AS was cultured with freshly isolated human monocytes in ultra low attachment tissue culture plates and treated with etanercept (Etan, 10 μg/mL) or control IgG (Ctrl, 10 μg/mL) for 24 hours. Cultures were imaged at 10× magnification using Nikon DS-Fi1/Elements Software. Scale bar 50 μm. (FIG. 26B) CHLA-255 was grown to 25% confluence in 12-well tissue culture plates and overlaid with freshly isolated human monocytes. Co-cultures were treated with etanercept (Etan) or IgG control (Ctrl) at 10 μg/mL for four days. Non-adherent cells were gently washed with PBS before mechanical dissociation to release adherent cells. Adherent CD14+ monocytes were quantified relative to NB from samples run in triplicate. **** p<0.0001;

FIGS. 27A-27D demonstrate etanercept treatment reduces tumor growth in SK-N-AS/monocyte xenogeneic mouse model. (FIG. 27A) SK-N-AS-luc and freshly isolated human monocytes were embedded in matrigel and injected into the subcutaneous right flank of eight-week female NSG mice. Mice were i.p. injected bi-weekly with 5 mg/kg (100 μg) etanercept (Etan) or IgG in a vehicle control (Ctrl) and tumor growth was tracked indirectly through luminescence imaging using IVIS imaging system. Representative luminescence images (Ctrl n=7, Etan n=13) measured in radiance (photons/second/cm2/steradian). (FIG. 27B) Graphical representation of total luminescence per tumor from (A) over time. (FIG. 27C) Mean tumor total luminescence over time, two-way ANOVA with Bonferroni multiple comparisons test (Ctrl n=7, Etan n=13); NB/Mon+Ctrl line is on top. (FIG. 27D) Representative tumors for each group following sacrifice at day 21. * p<0.05;

FIGS. 28A-28B demonstrate etanercept treatment alters murine TME. (FIG. 28A) Eukaryotic mRNA-seq was performed on murine component of tumor samples, and human transcripts were excluded from analysis by XenoFilterR program. Differentially regulated genes were identified using two-fold expression difference and adjusted P value<0.05 as cutoffs. (FIG. 28B) Over-representation gene-set enrichment analysis (GSEA) revealed pathways differentially regulated within the murine TME by etanercept (Etan) treatment compared to IgG control (Ctrl). Pathways listed are all non-redundant with ≥5 enriched genes and P>0.05;

FIG. 29 shows luminex data obtained from supernatants of monocytes-CHLA-255 co-cultures treated with etanercept (Etan) or control IgG (Ctrl) relative to monocyte only control (Mon.). Grey shading indicates cytokines that are upregulated during co-culture and reduced with Etan treatment in both donors. Two-way ANOVA with Tukey-corrected multiple comparisons between independent rows. n=3 biological replicates per condition, p<0.05 considered significant and indicated by bold red font. Etan=etanercept, Ctrl=IgG control.

### DETAILED DESCRIPTION

### I. Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

The terms “reduce,” “inhibit,” “diminish,” “suppress,” “decrease,” “prevent” and grammatical equivalents (including “lower,” “smaller,” etc.) or any antonym (such as “increase”, “activate”, etc.) when in reference to the expression of any symptom, gene, protein, and/or biomarker in an untreated individual relative to a treated subject, may mean that the quantity and/or magnitude of the symptoms, gene, protein, and/or biomarker in the treated subject is lower (or higher) than in the untreated subject by any amount that is recognized as clinically relevant by any person skilled in the art. In particular embodiments, the quantity and/or magnitude of the symptom(s), gene(s), protein(s), and/or biomarker(s) in the treated individual is at least 10% different than, at least 25% different than, at least 50% different than, at least 75% different than, and/or at least 90% different than the quantity and/or magnitude of the symptom(s), gene(s), protein(s), and/or biomarker(s) in the untreated subject.

As used herein, the term “therapeutically effective amount” is synonymous with “effective amount”, “therapeutically effective dose”, and/or “effective dose” and refers to the amount of compound that will elicit the biological or clinical response being sought by the practitioner in an individual in need thereof. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

As used herein, the terms “treatment,” “treat,” or “treating” refers to intervention in an attempt to alter the natural course of the individual or cell being treated, and may be performed either for prophylaxis or during the course of pathology of a disease or condition. Treatment may serve to accomplish one or more of various desired outcomes, including, for example, preventing occurrence or recurrence of disease, alleviation of symptoms, and diminishment of any direct or indirect pathological consequences of the disease, preventing metastasis, lowering the rate of disease progression, amelioration or palliation of the disease state, and remission or improved prognosis.

The term “individual” may be any individual and generally refers to an individual in need of a therapy. The individual can be a mammal, such as a human, dog, cat, horse, pig or rodent. The individual can be a patient, e.g., have or be suspected of having or at risk for having a disease or medical condition related to cancer and/or inflammation. For individuals having or suspected of having a medical condition directly or indirectly associated with cancer, the medical condition may be of one or more types, including neuroblastoma. The individual may have a disease or be suspected of having the disease. The individual may be asymptomatic. The individual may be of any gender.

As used herein, the term “nucleic acid” refers to one or more molecules comprising one or more nucleobases, nucleotides, and/or nucleosides. Nucleic acids encompassed herein may be of any length. Nucleic acids can be, for example, DNA, RNA, PNA, or combinations thereof. Nucleic acid derivatives may comprise one or more nucleic acids with any chemical modification on the nucleic acid backbone (including modifications to the phosphate linker and/or modifications to the nucleoside sugar) or to the nucleobase.

As used herein, the term “peptide” refers to one or more molecules comprising one or more amino acids, including polypeptides. Peptides encompassed herein may be of any length. The term “peptide”, in some embodiments encompassed herein, may be synonymous with “protein”, “proteoglycan”, “glycoprotein”, “antibody”, “fusion protein”, and/or “signaling peptide”. Peptide derivatives may be comprise one or more peptides with any chemical modification, including any post-translational modification. In some embodiments, the chemical modifications include (as non-limiting examples) disulfide linkages, methylation, hydroxylation, phosphorylation, acetylation, acylation, N-glycosylation, O-glycosylation, and/or O-GlcNacylation. In some embodiments, the peptide comprises at least one non-natural amino acid. In some embodiments, the peptide comprises non-peptide linkages in the backbone.

As used herein, the term “fusion protein” refers to a protein (or peptide) wherein at least two domains of the protein (or peptide) are encoded by separate genes. In some embodiments, there are multiple domains from the same gene, or from homologous genes. The genes may be from any species. In some embodiments, the genes are from the same species, such as from a human. In some embodiments, the fusion protein is translated from the same mRNA. In some embodiments, parts of the fusion protein (such as the domains of the fusion protein) are translated separately and linked post-translationally. In some embodiments, the domains of the fusion protein are linked by one or more chemical modifications, such as by one or more disulfide bridges.

As used herein, the term “tumor-promoting inflammation” refers to any signaling event, cytokine release, cytokine, production, cytokine binding, and/or reverse signaling, which promotes tumor progression, growth, metastasis, and/or transformation. Tumor-promoting inflammation may comprise the production, release, and/or binding of TNFα and/or IL-6. Tumor-promoting inflammation may activate NF-κB signaling and/or decrease IκBα.

Overview

The present disclosure concerns methods, systems, compositions, and kits for treatment of a medical condition related to TNF signaling, particularly wherein reduction in the level and/or activity and/or secretion of TNFα or downstream of TNFα signaling is therapeutically effective for an individual. The TNFa signaling may be modified such that when TNFa itself is inhibited and/or when a receptor of TNFa is inhibited, there is a reduction in the level of one or more inflammatory molecules, including for TNFα (secreted or membrane-bound), IL-6, and/or preventing NFkB activation.

### II. TNF Inhibitors

Embodiments of the disclosure encompass TNF inhibitors of any kind, including use thereof. The term “TNF inhibitor” encompasses one or more inhibitors that inhibit TNFα and also one or more inhibitors that inhibit any receptor of TNFα. In specific embodiments, the TNF inhibitor inhibitors the receptor TNFR1, the receptor TNFR2, or both. The inhibition by the inhibitor may be because of direct contact with TNFα and/or a receptor for TNFα, or the inhibition by the inhibitor may be because of indirect inhibition from direct contact with another entity that is not TNFα or a TNFα receptor.

The inhibitor for TNFα or a receptor of TNFα may be of any kind. In specific embodiments the inhibitor is a small molecule, immunotherapy, cell therapy, peptide, peptide derivative, antibody, fusion protein, glycoprotein, nucleic acid, nucleic acid derivative, or a combination thereof. In particular embodiment the inhibitor comprises an antibody, and the antibody may be of any kind including any immunologic binding agent such as IgG, IgM, IgA, IgD and IgE. The antibody may comprise any antibody-like molecule that has an antigen binding region, and includes antibody fragments such as Fab', Fab, F(ab')₂, single domain antibodies (DABs), Fv, scFv (single chain Fv), and the like. The antibody may be polyclonal or monoclonal.

The TNF inhibitor(s) may comprise etanercept, infliximab, certolizumab, golimumab, adalimumab, Thalidomide, lenalidomide, pomalidomide, a xanthine derivative (such as pentoxifylline), bupropion, 5-HT2A agonist hallucinogens (such as including (R)-DOI, TCB-2, LSD and LA-SS-Az), or a combination thereof. In some particular embodiments, the TNF inhibitor is etanercept.

Etanercept is a soluble form of the p75 TNF receptor fused to an Fc domain of a human IgG1 (TNFR:Fc). A commercially available etanercept is known as ENBREL® (Immunex Inc., Thousand Oaks, Calif.). Etanercept is produced by recombinant DNA technology in a Chinese hamster ovary (CHO) mammalian cell expression system. It consists of 934 amino acids and has an apparent molecular weight of approximately 150 kilodaltons (Physician's Desk Reference, 2002, Medical Economics Company Inc.). The full sequence expressed in CHO cells is shown below as SEQ ID NO:27. However, it is to be understood that minor modifications and deletions of this sequence (up to 1, 2, 3, 4, 5, 6, 7, 8, 9, 10, 11, 12, 13, 14, 15, 16, 17, 18, 19, 20%, or more) may be possible and can be used within the scope of the disclosed methods and compositions.

| | |
|---|---|
| 1 | Leu-Pro-Ala-Gln-Val-Ala-Phe-Thr-Pro-Tyr- |
| 11 | Ala-Pro-Glu-Pro-Gly-Ser-Thr-Cys-Arg-Leu- |
| 21 | Arg-Glu-Tyr-Tyr-Asp-Gln-Thr-Ala-Gln-Met- |
| 31 | Cys-Cys-Ser-Lys-Cys-Ser-Pro-Gly-Gln-His- |
| 41 | Ala-Lys-Val-Phe-Cys-Thr-Lys-Thr-Ser-Asp- |
| 51 | Thr-Val-Cys-Asp-Ser-Cys-Glu-Asp-Ser-Thr- |
| 61 | Tyr-Thr-Gln-Leu-Trp-Asn-Trp-Val-Pro-Glu- |
| 71 | Cys-Leu-Ser-Cys-Gly-Ser-Arg-Cys-Ser-Ser- |
| 81 | Asp-Gln-Val-Glu-Thr-Gln-Ala-Cys-Thr-Arg- |
| 91 | Glu-Gln-Asn-Arg-Ile-Cys-Thr-Cys-Arg-Pro- |
| 101 | Gly-Trp-Tyr-Cys-Ala-Leu-Ser-Lys-GlnGlu- |
| 111 | Gly-Cys-Arg-Leu-Cys-Ala-Pro-Leu-Arg-Lys- |
| 121 | Cys-Arg-Pro-Gly-Phe-Gly-Val-Ala-Arg-Pro- |
| 131 | Gly-Thr-Glu-Thr-Ser-Asp-Val-Val-CysLys- |
| 141 | Pro-Cys-Ala-Pro-Gly-Thr-Phe-Ser-Asn-Thr- |
| 151 | Thr-Ser-Ser-Thr-Asp-Ile-Cys-Arg-ProHis- |
| 161 | Gln-Ile-Cys-Asn-Val-Val-Ala-Ile-Pro-Gly- |
| 171 | Asn-Ala-Ser-Met-Asp-Ala-Val-Cys-Thr-Ser- |
| 181 | Thr-Ser-Pro-Thr-Arg-Ser-Met-Ala-Pro-Gly- |
| 191 | Ala-Val-His-Leu-Pro-Gln-Pro-Val-SerThr- |
| 201 | Arg-Ser-Gln-His-Thr-Gln-Pro-Thr-Pro-Glu- |
| 211 | Pro-Ser-Thr-Ala-Pro-Ser-Thr-Ser-Phe-Leu- |
| 221 | Leu-Pro-Met-Gly-Pro-Ser-Pro-Pro-Ala-Glu- |
| 231 | Gly-Ser-Thr-Gly-Asp-Glu-Pro-Lys-Ser-Cys- |
| 241 | Asp-Lys-Thr-His-Thr-Cys-Pro-Pro-Cys-Pro- |
| 251 | Ala-Pro-Glu-Leu-Leu-Gly-Gly-Pro-Ser-Val- |
| 261 | Phe-Leu-Phe-Pro-Pro-Lys-Pro-Lys-Asp-Thr- |
| 271 | Leu-Met-Ile-Ser-Arg-Thr-Pro-Glu-Val-Thr- |
| 281 | Cys-Val-Val-Val-Asp-Val-Ser-His-Glu-Asp- |
| 291 | Pro-Glu-Val-Lys-Phe-Asn-Trp-Tyr-Val-Asp- |
| 301 | Gly-Val-Glu-Val-His-Asn-Ala-Lys-Thr-Lys- |
| 311 | Pro-Arg-Glu-Glu-Gln-Tyr-Asn-Ser-Thr-Tyr- |
| 321 | Arg-Val-Val-Ser-Val-Leu-Thr-Val-Leu-His- |
| 331 | Gln-Asp-Trp-Leu-Asn-Gly-Lys-Glu-Tyr-Lys- |
| 341 | Cys-Lys-Val-Ser-Asn-Lys-Ala-Leu-Pro-Ala- |
| 351 | Pro-Ile-Glu-Lys-Thr-Ile-Ser-Lys-Ala-Lys- |
| 361 | Gly-Gln-Pro-Arg-Glu-Pro-Gln-Val-Tyr-Thr- |
| 371 | Leu-Pro-Pro-Ser-Arg-Glu-Glu-Met-Thr-Lys- |
| 381 | Asn-Gln-Val-Ser-Leu-Thr-Cys-Leu-Val-Lys- |
| 391 | Gly-Phe-Tyr-Pro-Ser-Asp-Ile-Ala-Val-Glu- |
| 401 | Trp-Glu-Ser-Asn-Gly-Gln-Pro-Glu-Asn-Asn- |
| 411 | Tyr-Lys-Thr-Thr-Pro-Pro-Val-Leu-Asp-Ser- |
| 421 | Asp-Gly-Ser-Phe-Phe-Leu-Tyr-Ser-Lys-Leu- |
| 431 | Thr-Val-Asp-Lys-Ser-Arg-Trp-Gln-Gln-Gly- |
| 441 | Asn-Val-Phe-Ser-Cys-Ser-Val-Met-His-Glu- |
| 451 | Ala-Leu-His-Asn-His-Tyr-Thr-Gln-Lys-Ser- |
| 461 | Leu-Ser-Leu-Ser-Pro-Gly-Lys |

In cases where combinations of TNF inhibitors are utilized, the combination may be of any suitable kind. The ratio of TNF inhibitors in a combination may be of any suitable kind, including at least 1:1, 1:2, 1:5, 1:10, 1:25, 1:50, 1:100, 1:1000, and so forth. In cases where three TNF inhibitors are utilized, the ratio may be, as examples, 1:1:1, 1:2:1, 1:1:2, 1:2:2, 1:5:1, 1:1:5, 1:5:5, 1:10:1, 1:1:10, 1:10:10, 1:50:1, 1:1:50, 1:50:50, 1:100:1, 1:1:100, 1:100:100, 1:1000:1, 1:1:1000, 1:1000:1000, and so forth. In some combinations, an inhibitor for TNFα is utilized in combination with an inhibitor of a TNFα receptor.

The TNF inhibitor(s) may be comprised in a pharmaceutical composition that comprises an effective amount of the TNF inhibitor(s) dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one TNF inhibitor will be known to those of skill in the art in light of the present disclosure, as exemplified by Remington: The Science and Practice of Pharmacy, 21′ Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The TNF inhibitor may comprise different types of carriers depending on whether it is to be administered in solid, liquid or aerosol form, and whether it need to be sterile for such routes of administration as injection. The compositions of the present disclosure can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The TNF inhibitor(s) may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the composition suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure may concern the use of a pharmaceutical lipid vehicle compositions that include TNF inhibitor(s), one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the TNF inhibitor(s) may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In particular embodiments of the present disclosure, the TNF inhibitor(s) are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, TNF inhibitor(s) may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

In some embodiments, disclosed herein is a method for treating neuroblastoma in a subject in need thereof by administering a therapeutically effective amount of a TNF inhibitor to the subject. In some embodiments, the TNF inhibitor is etanercept. In some embodiments, etanercept is administered to a subject subcutaneously. In some embodiments, the subject is a human patient, in some embodiments, the subject is a pediatric patient. In some embodiments, etanercept is administered to the subject at a dose in the range of 2-500 mg/dose, or 2-100 mg/dose or 10-80 mg/dose, either once/weekly or twice/weekly. In some embodiments, etanercept is administered at a dose of 25 mg/dose or 50 mg/does, either once/weekly or twice/weekly. In some embodiments, etanercept is administered at a dose of 0.8 mg/kg once/weekly or twice weekly.

C. Miscellaneous Pharmaceutical Compositions and Formulations

In other particular embodiments of the invention, the active compound TNF inhibitor(s) may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725, 871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### III. Treatment of Cancer and Reducing Tumor-Promoting Inflammation

Certain embodiments encompassed herein relate to methods for the treatment of cancer in an individual. The cancer may be of any type, including any tumor that expresses high levels of at least one TNFR and/or has high monocyte and/or tumor associated macrophage content, such as neuroblastoma (NB)). In cases wherein the cancer to be treated is neuroblastoma, which develops from immature nerve cells found in several areas of the body, the neuroblastoma may have arisen in and around the adrenal glands, in other areas of the abdomen, in the chest, in the neck, or near the spine, where groups of nerve cells exist. Neuroblastoma affects children in particular, and so in some embodiments the affected individual is 10, 9, 8, 7, 6, 5, 4, 3, 2, 1, or less than 1 year of age. The individual may be a child or infant. The individual may or may not have a family history of neuroblastoma. The neuroblastoma may or may not have metastasized, such as to the lymph nodes, bone marrow, liver, skin and/or bones. In some embodiments, the neuroblastoma has resulted in spinal cord compression. In some embodiments, the neuroblastoma cells may secrete certain compounds that irritate other normal tissues, causing signs and symptoms called cause paraneoplastic syndromes.

Embodiments of the disclosure include methods of treating cancer, comprising administering to an individual with cancer a therapeutically effective amount of one or more TNF inhibitors. The TNF inhibitor(s) may inhibit TNF and/or one or more TNF receptors in cancer cells and/or in cells in the tumor microenvironment. The methods encompass one or more administrations of one or more TNF inhibitors. In specific embodiments, the cancer is neuroblastoma and the one or more TNF inhibitors are administered multiple times to the individual in need. In specific cases, the individual is a child with neuroblastoma and one or more TNF inhibitors, including TNFα inhibitors, are administered to the child.

In particular embodiments, the cancer cells express one or more cytokine receptors, such as tumor necrosis factor (TNF) receptors (TNFRs) and/or IL-6 receptors, for example. The cancer may be activated by tumor-promoting inflammation such as cytokines, including TNFα and/or IL-6. The cytokines may bind to receptors on cancer cells and activate signaling pathways, which may include, for example, NF-κB and/or Stat3 signaling. The cytokines may decrease the expression and/or levels of IκBα in the cancer cell. In some embodiments, the cancer is activated by monocytes and/or macrophages. In certain embodiments, monocytes and/or macrophages promote growth of cancer cells such as by activating NF-κB and/or Stat3 signaling through TNF signaling and/or IL-6 signaling. The monocytes and/or macrophages may activate and/or promote growth of cancer via membrane-bound TNFα found on the surface of the monocyte and/or macrophage. In some embodiments, the individual is administered a therapy, such as at least one small molecule, immunotherapy, cell therapy, peptide, peptide derivative, antibody, fusion protein, glycoprotein, nucleic acid, nucleic acid derivative, or a combination thereof, which is capable of inhibiting any TNF molecule and/or signaling pathway. The individual may be administered a therapeutically effective amount of the therapy, as determined by one skilled in the art. In some embodiments, the therapy comprises at least one fusion protein, such as an artificially engineered fusion protein, and/or an antibody. In some embodiments, the fusion protein comprises at least one cytokine-binding domain, single-chain variable fragment (scFv), and/or variable region domain. Such domains may be capable of binding one or more cytokines, including cytokines that contribute to tumor-promoting inflammation, such as TNFα and/or IL-6. In some embodiments, the antibody is capable of binding one or more cytokines, including cytokines that contribute to tumor-promoting inflammation, such as TNFα and/or IL-6. In some embodiments, the therapy comprises etanercept, infliximab, certolizumab, golimumab, adalimumab, or a combination thereof.

For any method encompassed herein, the compositions of the present disclosure can be administered to an individual in need thereof in any suitable manner. Examples include at least intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), and so forth.

In some embodiments, the one or more TNF inhibitor is administered, either concurrently or sequentially, with one or more therapies for treating neuroblastoma. Therapies for treating neuroblastoma are known by those skilled in the art and include surgery removing the tumor, radiation therapy, chemotherapy or combinations thereof. In such embodiments, the TNF inhibitor can be administered to the individual either before, at the same time and/or after a therapy for treating neuroblastoma.

In some embodiments, tumor-promoting inflammation is reduced in an individual by contacting cells (including any cell, such as any cancer cell, encompassed herein) and/or extracellular fluid (such as blood, plasma, serum, interstitial fluid, tumor interstitial fluid, cell culture fluid, or a combination thereof) with any composition(s) encompassed herein. Tumor-promoting inflammation may be reduced by decreasing the expression, production, and/or levels of one or more inflammatory cytokines, such as soluble TNFα, membrane-bound TNFα, and/or IL-6, for example. Reducing tumor-promoting inflammation may comprise increasing IκBα expression and/or levels, including in cancer cells. The contacting may occur in vitro, in vivo, and/or ex vivo.

Embodiments of the disclosure include methods of reducing TNFα production in cancer cells, including NB cells. In such cases, cancer cells are subject to one or more TNF inhibitors in an effective amount to reduce TNFα production in the cancer cells. Embodiments of the disclosure also include methods of reducing IL-6 production in cancer cells, including NB cells. In such cases, cancer cells are subject to one or more TNF inhibitors in an effective amount to reduce IL-6 production in the cancer cells.

When multiple TNF inhibitors are administered, the different inhibitors may or may not be in the same formulation. The different inhibitors may or may not be administered to an individual at the same time. A first TNF inhibitor may be administered prior to a second TNF inhibitor. A first TNF inhibitor may be administered prior to and at the same time as a second TNF inhibitor. When there are multiple administrations of TNF inhibitors, whether or not the subsequent inhibitor(s) is the same as the initial inhibitor that is administered, the duration between administrations may be of any length of time, including within 1-24 hours, 1-7 days, 1-4 weeks, 1-12 months, or longer, and including any subrange therebetween. In some cases, a TNFα inhibitor is administered one or more times to an individual, and following this a TNFR inhibitor is administered one or more times to an individual, or vice versa. This change in course of action may or may not be caused by a recurrence and/or metastasis of the cancer.

Gene Disruption

In some embodiments, cancer cells in an individual may be targeted for genetic modification to decrease or eliminate expression of TNFα and one or more cytokine receptors, including TNFR1 and/or TNFR2. The genetic modification may be performed using any method known in the art, such as CRISPR-Cas9 or RNAi. The sgRNA sequences used may be any sequence sufficient to knocking out, TNFα, TNFR1 and/or TNFR2, including for example sgRNA sequences that target SEQ ID NOs:1-6.

SEQ ID NO: 1
GCCGTGGGTCAGTATGTGAGAGG
SEQ ID NO: 2
GGAGTGATCGGCCCCCAGAGGG
SEQ ID NO: 3
GGAGGAGACACCATCAAGAGAGG
SEQ ID NO: 4
GGGGCACTGGATGGTGGCATGGG
SEQ ID NO: 5
GGGGCGGGGACCTGGGCATCAGG
SEQ ID NO: 6
GGCCCATACTGCCCAGCCGGTGG

SEQ ID NOs:1-2 comprise sequences found in the gene encoding TNFα. SEQ ID NOs:3-4 comprise sequences found in the gene encoding TNFR1. SEQ ID NOs:5-6 comprise sequences found in the gene encoding TNFR2.

The sgRNA sequences may comprise a T7 promoter, guide sequence, and gRNA scaffold. The T7 promoter may comprise the sequence of ttaatacgactcactata (SEQ ID NO:28). The gRNA scaffold may comprise the sequence of gttttagagctagaaatagc. In some embodiments, the sgRNA sequence comprise SEQ ID NO:7, SEQ ID NO:8, SEQ ID NO:9, SEQ ID NO:10, SEQ ID NO:11, SEQ ID NO:12, or a combination thereof.

SEQ ID NO: 7
ttaatacgactcactataGGCGTGGGTCAGTATGTGAGgtttt
agagctagaaatagc
SEQ ID NO: 8
ttaatacgactcactataGGAGTGATCGGCCCCCAGAgtttta
gagctagaaatagc
SEQ ID NO: 9
ttaatacgactcactataGGAGGAGACACCATCAAGAGgtttt
agagctagaaatagc
SEQ ID NO: 10
ttaatacgactcactataGGGGCACTGGATGGTGGCATgtttt
agagctagaaatagc
SEQ ID NO: 11
ttaatacgactcactataGGGGCGGGGACCTGGGCATCgtttt
agagctagaaatagc
SEQ ID NO: 12
ttaatacgactcactataGGCCCATACTGCCCAGCCGGgtttt
agagctagaaatagc

SEQ ID NOs:7-8 comprise sgRNA sequences capable of targeting TNFα. SEQ ID NOs:9-10 comprise sequences capable of targeting TNFR1. SEQ ID NOs:11-12 comprise sequences capable of tageting TNFR2.

In some embodiments, the gene disruption for TNFα, TNFR1, and TNFR2 (as examples) is carried out by effecting a disruption in the gene, such as a knock-out, insertion, missense or frameshift mutation, such as biallelic frameshift mutation, deletion of all or part of the gene, e.g., one or more exons or portions therefore, and/or knock-in. For example, the disruption can be effected be sequence-specific or targeted nucleases, including DNA-binding targeted nucleases such as zinc finger nucleases (ZFN) and transcription activator-like effector nucleases (TALENs), and RNA-guided nucleases such as a CRISPR-associated nuclease (Cas), specifically designed to be targeted to the sequence of the gene or a portion thereof.

In some embodiments, the disruption is transient or reversible, such that expression of the gene is restored at a later time. In other embodiments, the disruption is not reversible or transient, e.g., is permanent.

In some embodiments, gene disruption of TNFα, TNFR1, and/or TNFR2 is carried out by induction of one or more double-stranded breaks and/or one or more single-stranded breaks in the gene, typically in a targeted manner. In some embodiments, the double-stranded or single-stranded breaks are made by a nuclease, e.g., an endonuclease, such as a gene-targeted nuclease. In some aspects, the breaks are induced in the coding region of the gene, e.g., in an exon. For example, in some embodiments, the induction occurs near the N-terminal portion of the coding region, e.g., in the first exon, in the second exon, or in a subsequent exon.

The target cell may be introduced to a guide RNA and CRISPR enzyme, or mRNA encoding the CRISPR enzyme. In some aspects, the cell is introduced to 1, 2, 3, 4, 5, or more guide RNAs simultaneously. For example, the cell may be introduced to 1, 2, or 3 guide RNAs during a first electroporation and then further introduced to 1, 2, or 3 additional guide RNAs during a second electroporation, and so forth.

In some embodiments, gene disruption of TNFα, TNFR1, and/or TNFR2 is achieved using antisense techniques, such as by RNA interference (RNAi), short interfering RNA (siRNA), short hairpin (shRNA), and/or ribozymes are used to selectively suppress or repress expression of the gene. siRNA technology is RNAi that employs a double-stranded RNA molecule having a sequence homologous with the nucleotide sequence of mRNA that is transcribed from the gene, and a sequence complementary with the nucleotide sequence. siRNA generally is homologous/complementary with one region of mRNA that is transcribed from the gene, or may be siRNA including a plurality of RNA molecules that are homologous/complementary with different regions. In some aspects, the siRNA is comprised in a polycistronic construct.

In some embodiments, the disruption of TNFα, TNFR1, and/or TNFR2 is achieved using a DNA-targeting molecule, such as a DNA-binding protein or DNA-binding nucleic acid, or complex, compound, or composition, containing the same, which specifically binds to or hybridizes to the gene. In some embodiments, the DNA-targeting molecule comprises a DNA-binding domain, e.g., a zinc finger protein (ZFP) DNA-binding domain, a transcription activator-like protein (TAL) or TAL effector (TALE) DNA-binding domain, a clustered regularly interspaced short palindromic repeats (CRISPR) DNA-binding domain, or a DNA-binding domain from a meganuclease. Zinc finger, TALE, and CRISPR system binding domains can be engineered to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data.

For CRISPR-mediated disruption of TNFα, TNFR1, and/or TNFR2, the guide RNA and endonuclease may be introduced to the target cells by any means known in the art to allow delivery inside cells or subcellular compartments, and agents/chemicals and/or molecules (proteins and nucleic acids) that can be used include liposomal delivery means, polymeric carriers, chemical carriers, lipoplexes, polyplexes, dendrimers, nanoparticles, emulsion, natural endocytosis or phagocytose pathway as non-limiting examples, as well as physical methods, such as electroporation. In specific aspects, electroporation is used to introduce the guide RNA and endonuclease, or nucleic acid encoding the endonuclease.

In some embodiments, the alteration of the expression, activity, and/or function of the of TNFα, TNFR1, and/or TNFR2 gene is carried out by disrupting the gene of TNFα, TNFR1, and/or TNFR2. In some aspects, the gene is modified so that its expression is reduced by at least at or about 10, 20, 30, or 40%, generally at least at or about 50, 60, 70, 80, 90, 91, 92, 93, 94, 95, 96, 97, 98, 99, or 100% as compared to the expression in the absence of the gene modification or in the absence of the components introduced to effect the modification.

In some embodiments, the alteration is transient or reversible, such that expression of the gene is restored at a later time if desired. In other embodiments, the alteration is not reversible or transient, e.g., is permanent.

In some embodiments, gene alteration is carried out by induction of one or more double-stranded breaks and/or one or more single-stranded breaks in the gene, typically in a targeted manner. In some embodiments, the double-stranded or single-stranded breaks are made by a nuclease, e.g. an endonuclease, such as a gene-targeted nuclease. In some aspects, the breaks are induced in the coding region of the gene, e.g. in an exon. For example, in some embodiments, the induction occurs near the N-terminal portion of the coding region, e.g. in the first exon, in the second exon, or in a subsequent exon.

In some aspects, the double-stranded or single-stranded breaks undergo repair via a cellular repair process, such as by non-homologous end-joining (NHEJ) or homology-directed repair (HDR). In some aspects, the repair process is error-prone and results in disruption of the gene, such as a frameshift mutation, e.g., biallelic frameshift mutation, which can result in complete knockout of the gene. For example, in some aspects, the disruption comprises inducing a deletion, mutation, and/or insertion. In some embodiments, the disruption results in the presence of an early stop codon. In some aspects, the presence of an insertion, deletion, translocation, frameshift mutation, and/or a premature stop codon results in disruption of the expression, activity, and/or function of the gene.

In some embodiments, the alteration is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

### IV. Kits

Any of the TNF inhibitor compositions described herein may be comprised in a kit. The kits may comprise a suitably aliquoted TNF inhibitor of the present disclosure. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the TNF inhibitor(s) and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. The TNF inhibitor compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

Irrespective of the number and/or type of containers, the kits of the disclosure may also comprise, and/or be packaged with, an instrument for assisting with the injection/administration and/or placement of the ultimate composition within the body of an animal. Such an instrument may be a syringe, pipette, forceps, and/or any such medically approved delivery vehicle.

### EXAMPLES

The following examples are included to demonstrate certain non-limiting aspects of the disclosure. It should be appreciated by those of skill in the art that the techniques disclosed in the examples that follow represent techniques discovered by the inventors to function well in the practice of the disclosed subject matter. However, those of skill in the art should, in light of the present disclosure, appreciate that many changes can be made in the specific embodiments that are disclosed and still obtain a like or similar result without departing from the spirit and scope of the disclosed subject matter.

### I. Example 1: General Embodiments

In analysis of clinical outcome-linked NB gene expression data, high tumor expression of TNFR1 showed a strong correlation with poor event-free survival (5-year EFS 15% vs 70%), highlighting the potential importance of TNFα signaling in neuroblastoma (NB). Using TNFα knockout human NB cell lines, it was determined that monocytes, but not NB cells, produce TNFα during in vitro co-culture. Furthermore, using a TNFα converting enzyme inhibitor to prevent secretion of soluble TNFα and CRISPR/Cas9 generated knockouts (KOs) of TNFR1 and TNFR2 in NB, it was found that NB-derived TNFR2 and monocyte-derived mTNFα were required for NF-κB pathway activation in monocytes that led to production of pro-inflammatory IL-6, a known NB growth factor. In contrast, NB-derived TNFR1 required monocyte-derived sTNFα for downstream activation of NF-κB signaling in NB cells. Neutralization of TNFα, but not TNFR1 or TNFR2, prevented NF-κB activation in both NB cells and monocytes and that it reduced IL-6 production up to 60% compared to controls.

The embodiments encompassed herein demonstrate that TNFR1-expressing NB cells activate monocytes via contact-dependent mTNFα signaling, leading to NF-κB activation and production of IL-6, a known driver of NB progression and therapy resistance. These findings reveal a novel, druggable mechanism of tumor-promoting inflammation in NB.

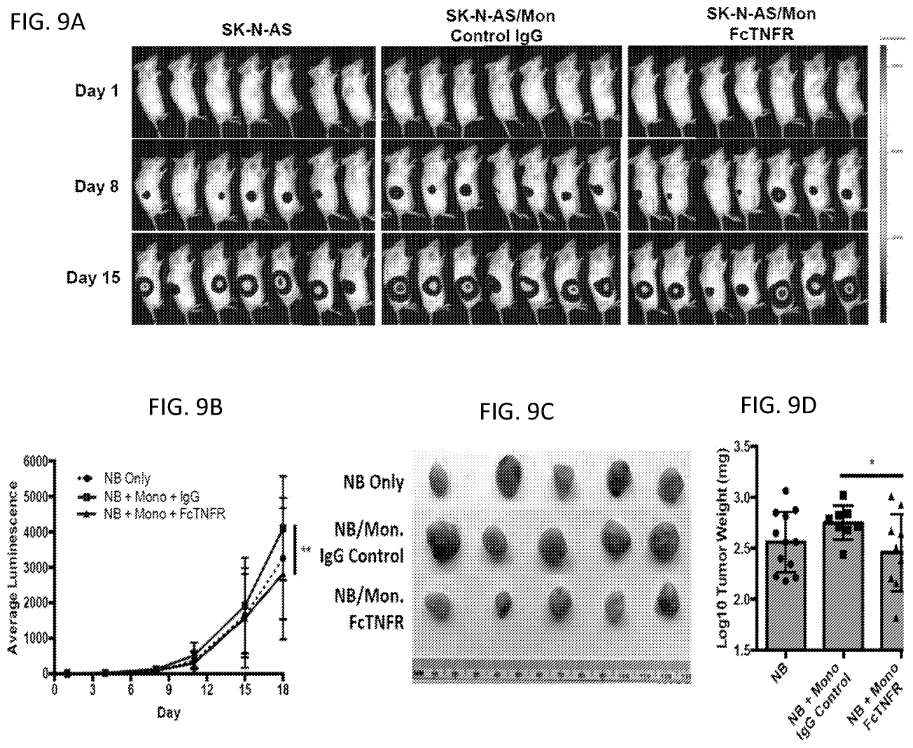

In embodiments encompassed herein, etanercept eliminates tumor-promoting inflammation, which provides motivation for an effective immunotherapy of cancer, including neuroblastoma. TNFR2 expression by neuroblastoma reverse signals through mTNFα on monocytes leading to downstream activation of NF-kB. Activation of NF-kB in monocytes leads to production of tumor-promoting cytokines like IL-6 which activates downstream STAT3 in NB, as well as sTNF which activates NF-kB in NB through TNFR1. Together these functions promote increased NB survival and proliferation. Etanercept effectively neutralizes both TNF isoforms, blocking monocyte NF-kB activation and IL-6 production, reducing tumor growth (FIG. 9).

### II. Example 2: NB Induces Monocyte IL-6 Production in Contact and NF-kB Dependent Mechanism

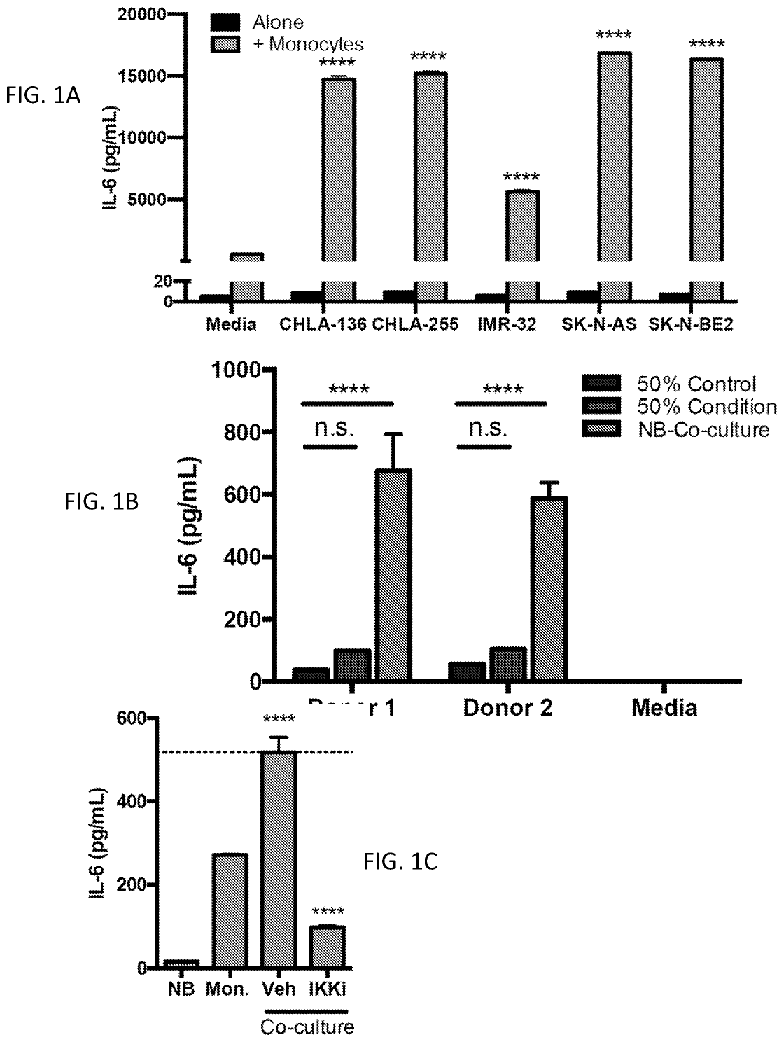

NB cell lines were co-cultured with monocytes in a 4:1 ratio. Supernatant analysis showed that IL-6 production was significantly increased when NB cells were co-cultured with monocytes compared to NB cells or monocytes cultured alone (FIG. 1A). Monocytes cultured in NB conditioned media did not increase production of IL-6 compared to control media, whereas monocytes cultured directly with NB cells did increase IL-6 production (FIG. 1B). An IKK inhibitor reversed IL-6 production in NB-monocyte co-cultures (FIG. 1C).

### III. Example 3: NB Expresses Functional TNFRs

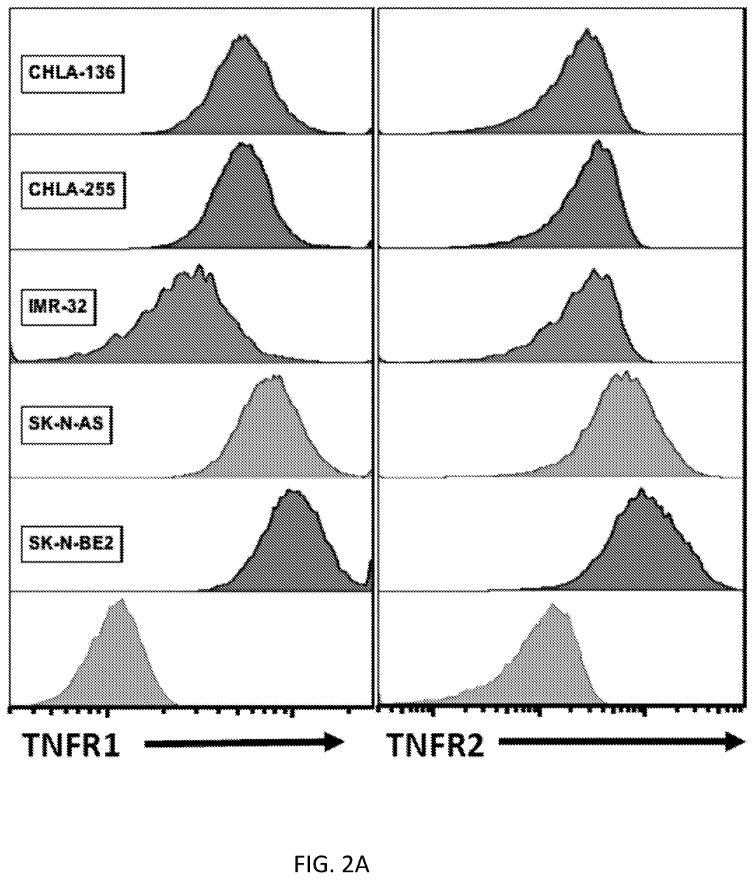

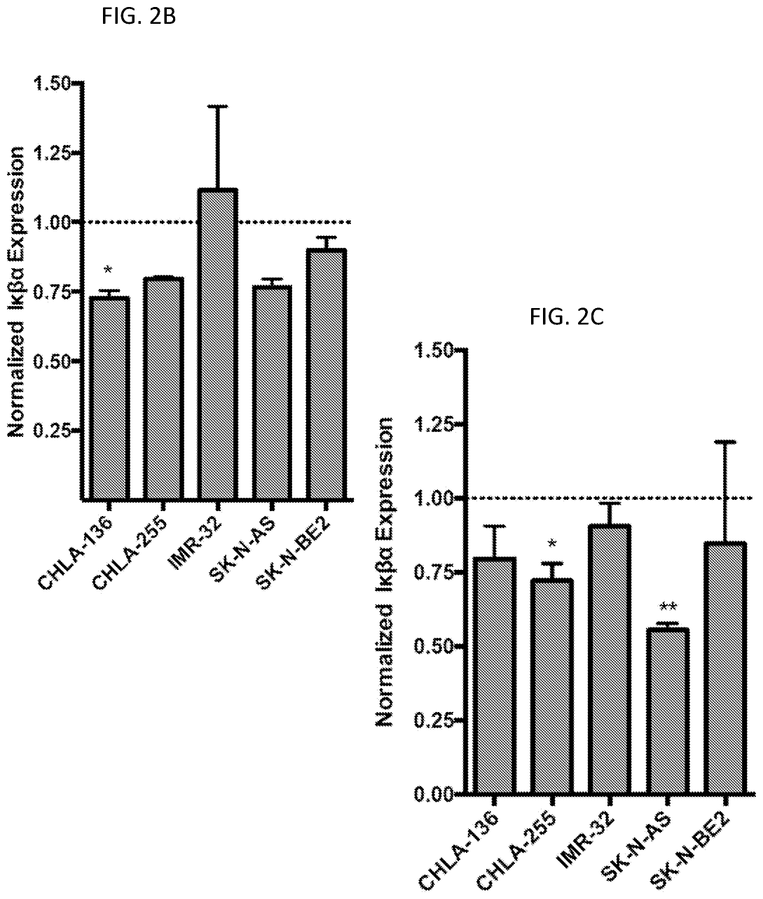

All tested NB cell lines expressed both TNFR1 and TNFR2 (FIG. 2A). NB cell lines treated with rhTNFα (FIG. 2B) or co-cultured with monocytes (FIG. 2C) showed a decreasing trend of IκBα expression compared to untreated NB cells.

### IV. Example 4: TNFR1 and TNFR2 can be Successfully Knocked Out Using CRISPR/Cas9 Gene Editing

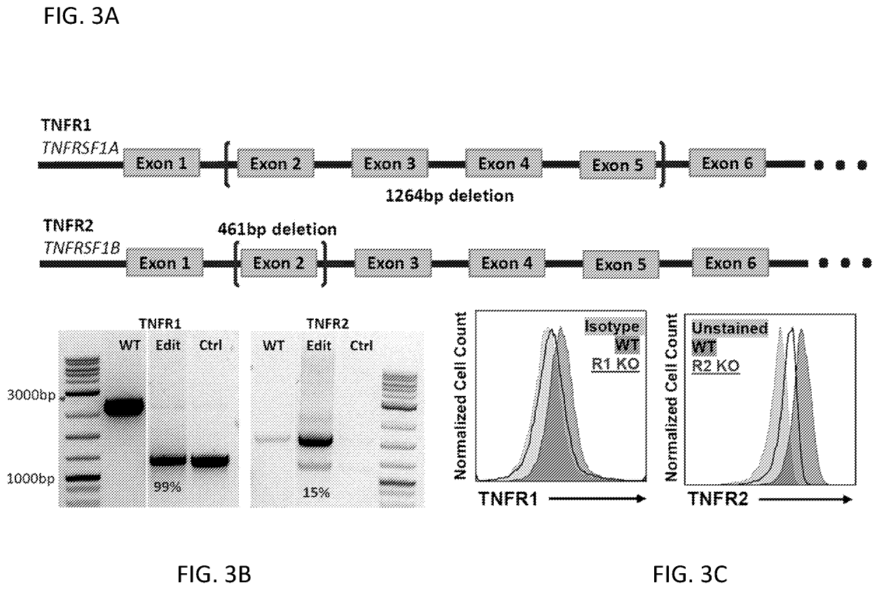

TNFR1 and TNFR2 were knocked out of NB cells using CRISPR/Cas9 gene editing. Exons 2-5 were removed from the gene encoding TNFR1 and exon 2 was removed from the gene encoding TNFR2 (FIG. 3A). PCR confirmed a 99% and 15% editing efficiency in TNFR1 and TNFR2 respectively (FIG. 3B). Single clones were expanded and assayed for expression of the receptors. Flow cytometry revealed successful knockout of both TNFR1 and TNFR2 in the respective clonal cell populations (FIG. 3C).

### V. Example 5: NB TNFR2 is Critical for Monocyte Activation

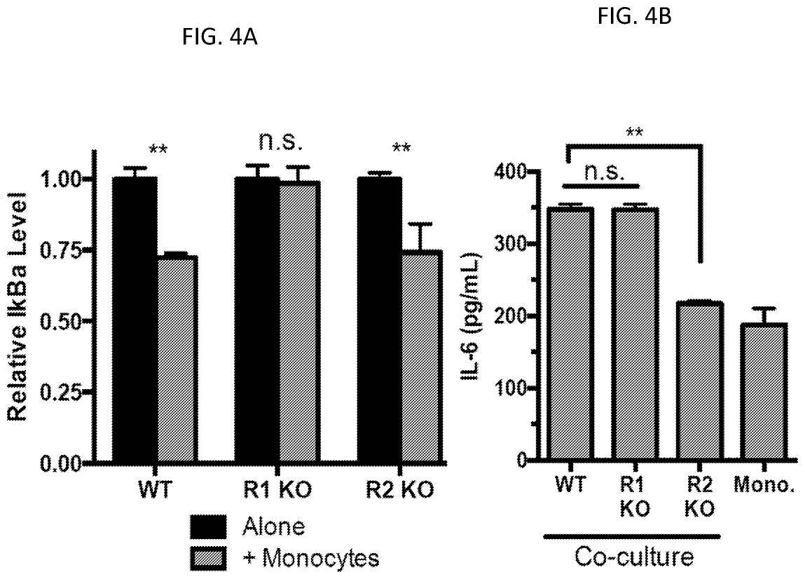

TNFR1 knockout (KO), TNFR2 KO, and wild-type NB cells were cultured alone or co-cultured with monocytes. IκBα levels were reduced in wild-type and TNFR2 KO cells when cultured with monocytes, while TNFR1 KO cells remained unchanged (FIG. 4A). IL-6 levels were increased in co-cultures of monocytes with wild-type and TNFR1 KO NB cells, while co-cultures of monocytes with TNFR2 KO NB cells showed levels of IL-6 near the levels found in monocytes cultured alone (FIG. 4B).

### VI. Example 6: Monocyte mTNFα is Critical for Monocyte Activation

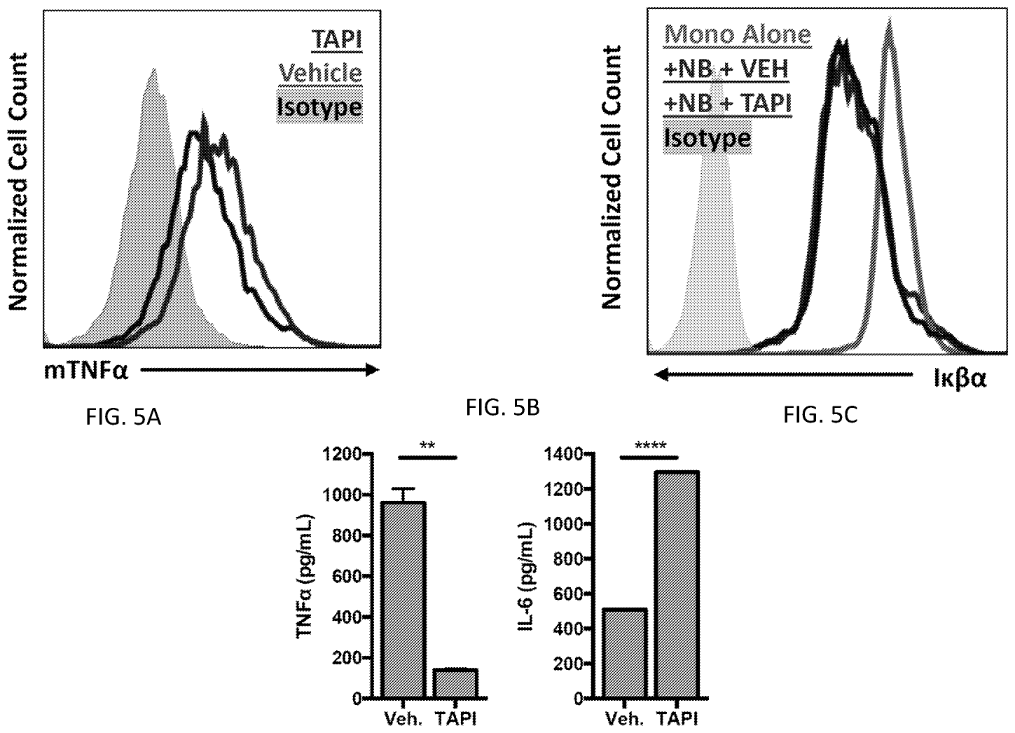

Co-culture of NB cells and monocytes, when cultured with TAPI (which decreases the conversion of membrane-bound TNFα to soluble TNFα), increased the activation of monocytes as measured by IL-6 levels as compared to a vehicle control (FIG. 5A, 5B). TAPI had no effect on intracellular IκBα expression in monocytes (FIG. 5C).

### VII. Example 7: Research-Grade TNF Neutralizing Antibodies Reduce Tumor-Promoting Inflammation

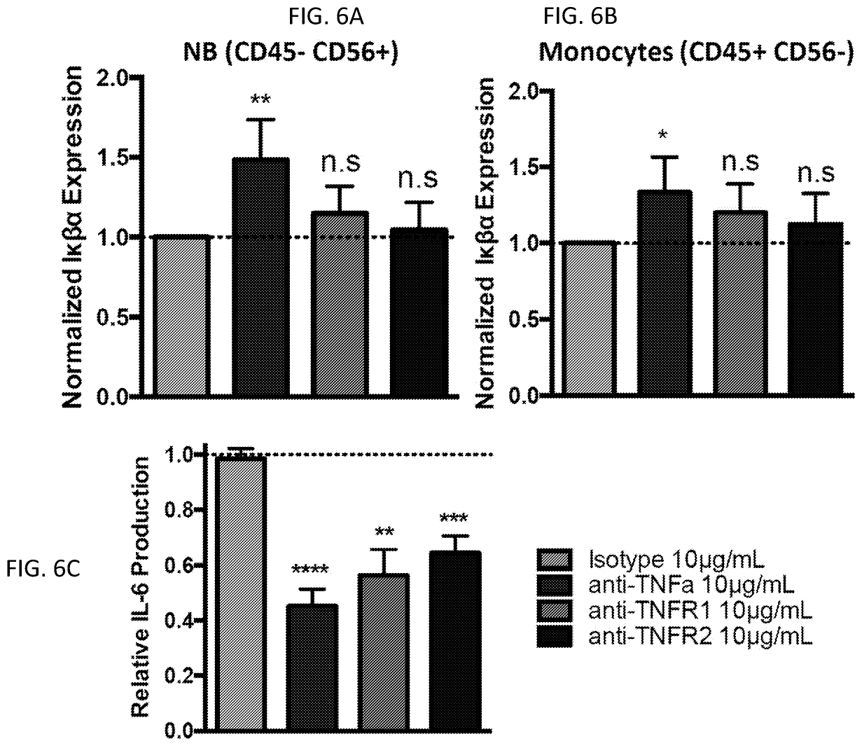

Co-cultures of NB cells and monocytes in the presence of TNFα, TNFR1, TNFR2, or isotype control antibodies revealed that blocking TNFα increased IκBα levels in both NB cells and monocytes compared to the isotype control (FIG. 6A, 6B). All three TNF antibodies decreased IL-6 production compared to the isotype control (FIG. 6C).

### VIII. Example 8: Etanercept Disrupts Tumor Promoting Inflammation, In Vitro

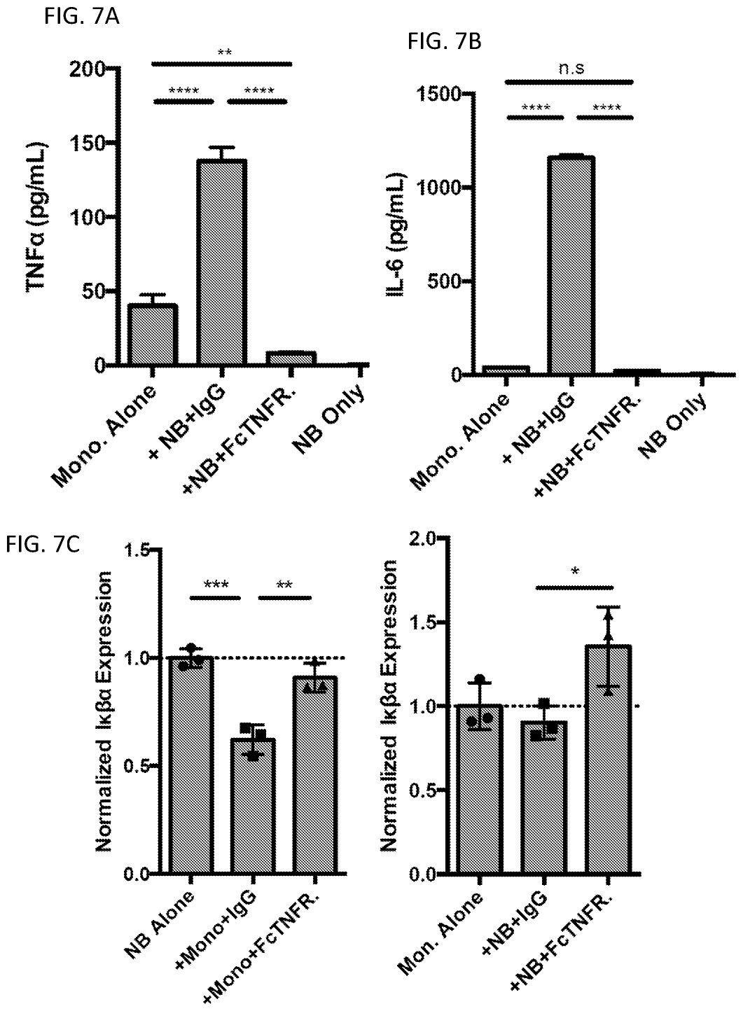

The NB cell line, SK-N-AS, co-cultured with monocytes and treated with the TNF inhibitor, etanercept, showed a decrease in TNFα and IL-6 levels compared to an IgG control. (FIG. 7A). Etanercept also restored IκBα levels to the levels found in NB cells cultured alone, while increasing monocyte levels of IκBα (FIG. 7B, 7C).

### IX. Example 9: Etanercept Reduces Monocyte-Induced NB Growth In Vitro

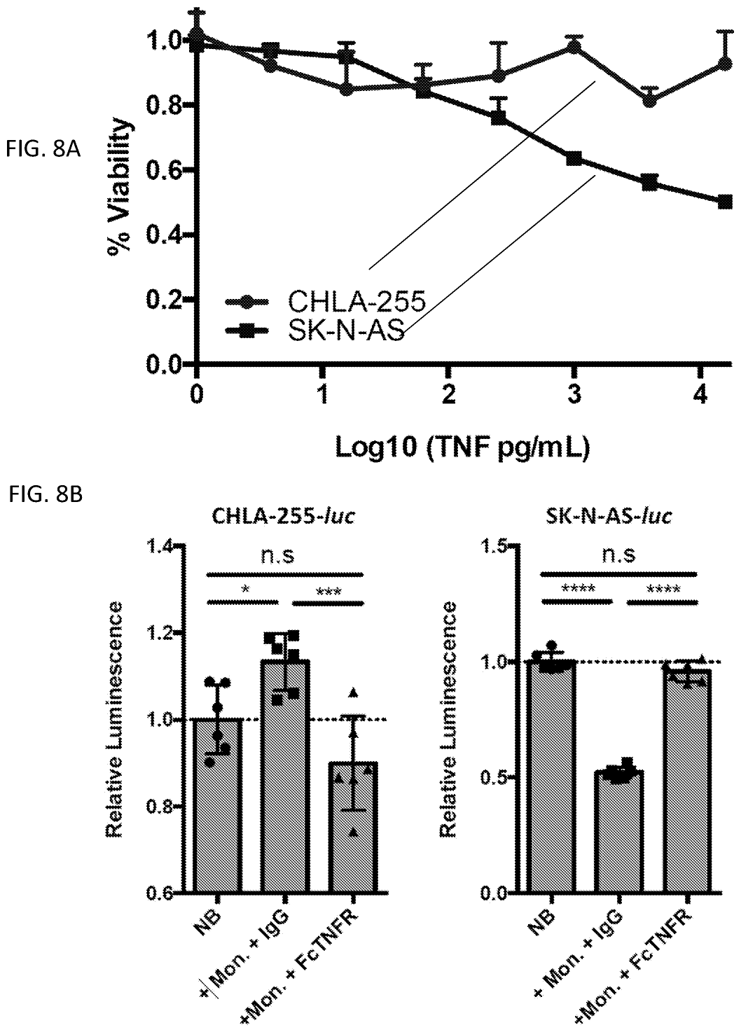

The NB cell lines CHLA-255 and SK-N-AS were modified to express luciferase (CHLA-255-luc and SK-N-AS-luc, respectively). CHLA-255-luc remained viable with increasing rhTNF concentration, whereas SK-N-AS-luc viability decreased with increasing rhTNF concentration (FIG. 8A). In specific embodiments, the difference between the two cells lines originates from the addition of monocytes. TNF can induce both pro-survival signaling and pro-death signaling through the same receptor. In the TNF curve, CHLA-255 does not undergo cell death, while SK-N-AS does. When this is applied to the co-culture, monocytes produce a baseline level of TNF and increased levels when co-cultured with NB. Therefore, the effect seen between the two lines in the IgG condition is not the IgG but because of the addition of monocytes and the cytokines they produce. With CHLA-255, there is no cell death, so the cells appear to proliferate more above the baseline, and etanercept reduces this. In SK-N-AS, the TNF produced by the monocytes is likely sufficient to cause cell death in the IgG condition, and that is rescued by adding etanercept, in at least certain embodiments.

Etanercept reversed the effect of monocyte-induced TNF signaling on NB cells (FIG. 8B).

### X. Example 10: Etanercept Treatment Eliminates Monocyte-Induced Tumor Growth In Vivo

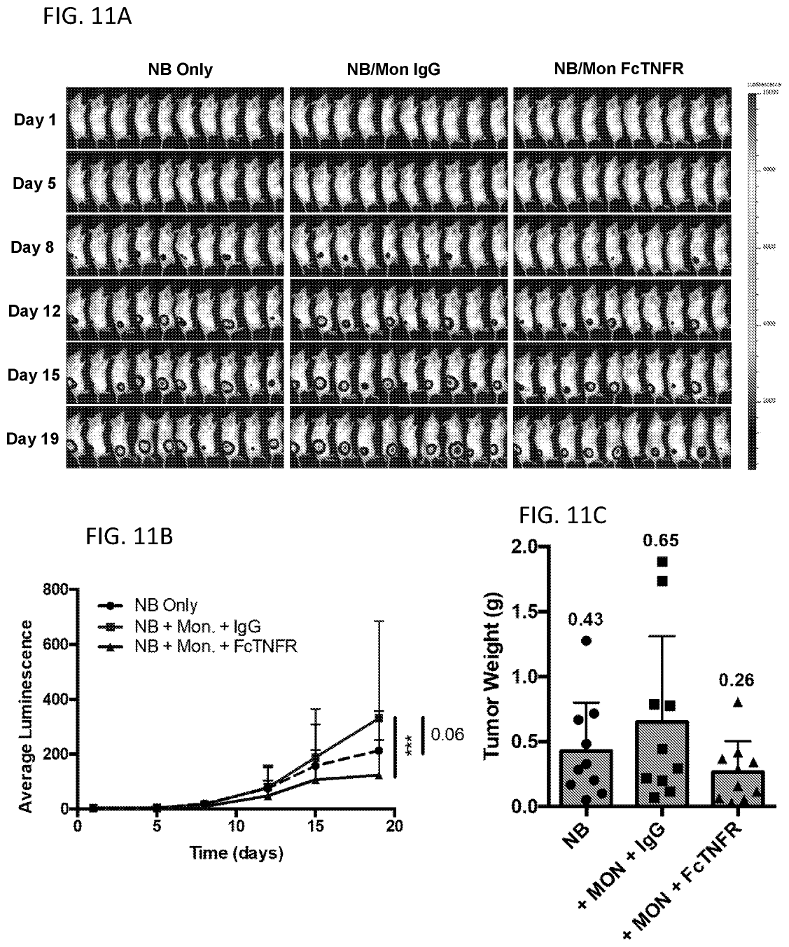

NSG mice were injected with luciferase-labeled NB cells (SK-N-AS) with and without monocytes embedded in Matrigel. Tumors were allowed to grow while being treated bi-weekly with 100 μg of etanercept or an IgG control. Etanercept treatment significantly decreased the growth of tumors compared to the IgG control (FIG. 9A-9C). A subsequent, study using different NB cells (CHLA-255) and different monocytes showed similar results (FIG. 11A-C).

### XI. Example 11: NB Activates Monocytes in a Cell Contact- and TNFα-Dependent Manner

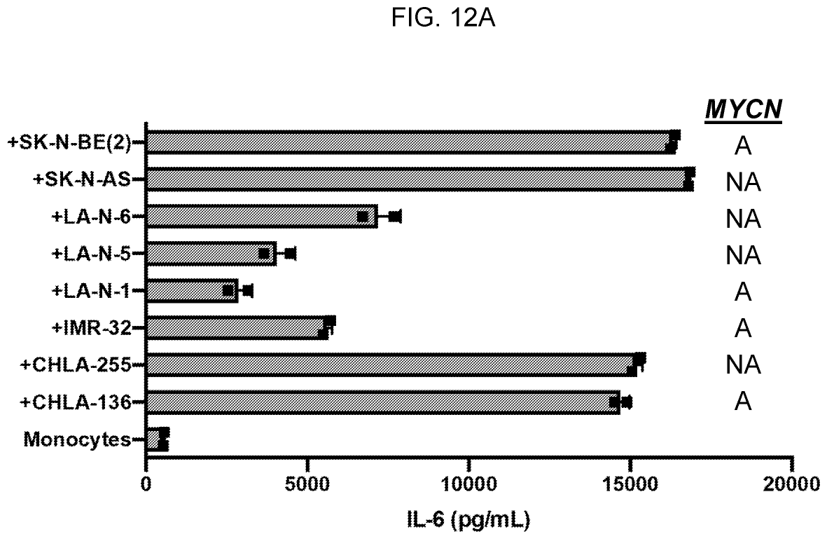

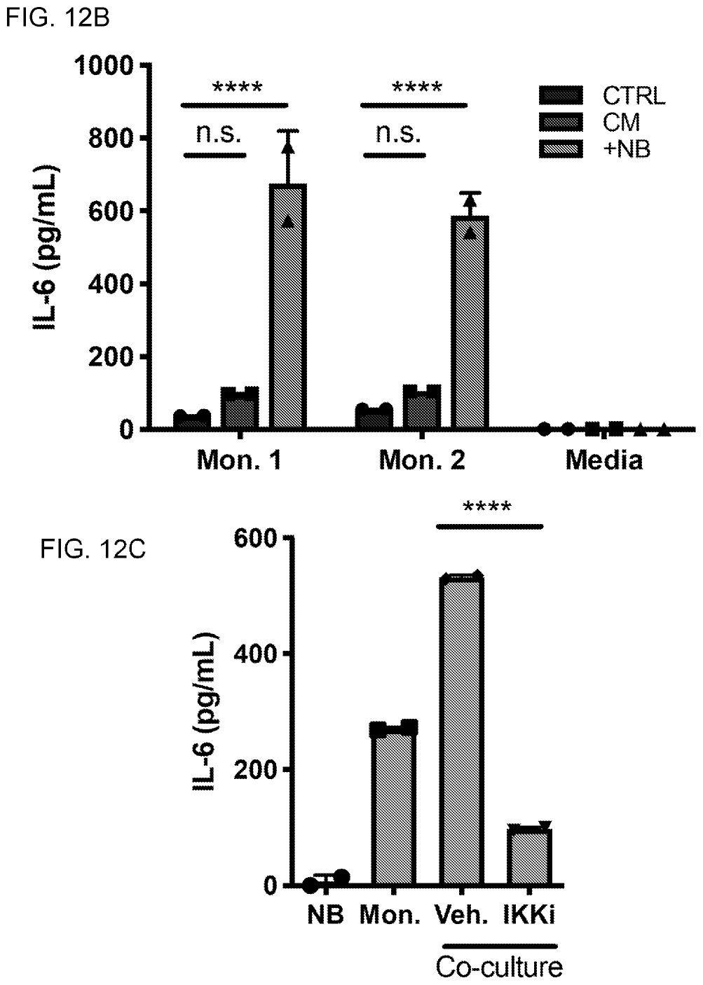

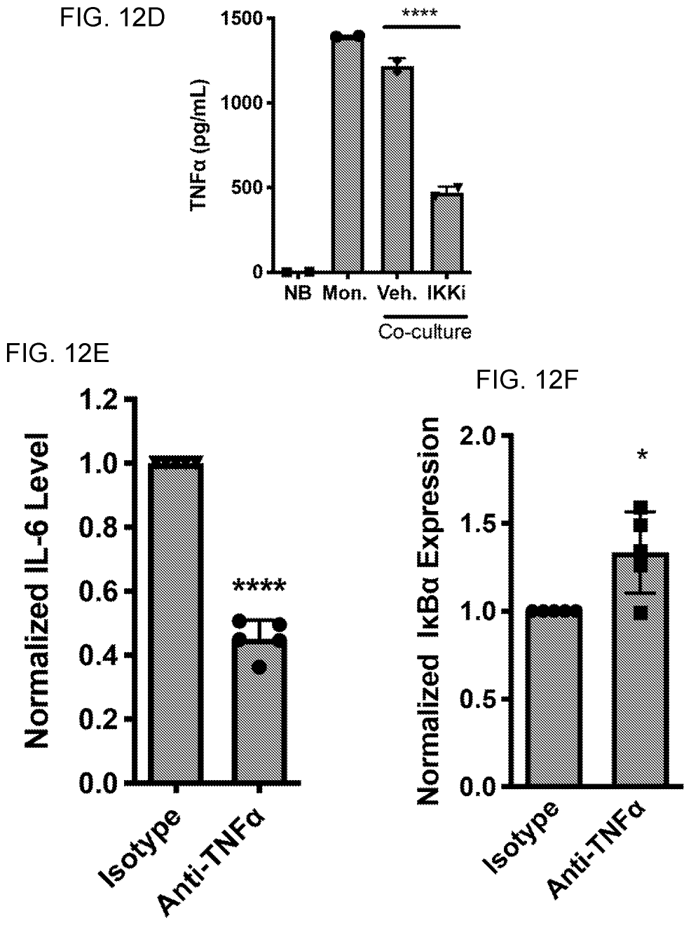

Previous studies demonstrated that co-culturing monocytes with CHLA-255 MYCN-non-amplified NB cells induces monocytes to produce IL-6 (9). To determine whether this response relates to the MYCN amplification status of NB cells, monocytes were co-cultured with four MYCN-amplified (A) and four non-amplified (NA) NB cell lines and found that all lines induced monocytes to produce IL-6 regardless of MYCN status (P<0.001, FIG. 12A). To examine whether NB cells and monocytes must be in direct contact to elicit IL-6 production, monocytes from two donors were either placed in co-culture with CHLA-255 NB cells or cultured alone in medium that had been conditioned for one week with CHLA-255 cells. After 24 hours, monocytes in the conditioned medium did not produce significantly more IL-6 than those cultured in control medium. In contrast, monocytes co-cultured with CHLA-255 cells strongly upregulated IL-6 production, suggesting that the effect is contact-dependent (P<0.0001, FIG. 12B).

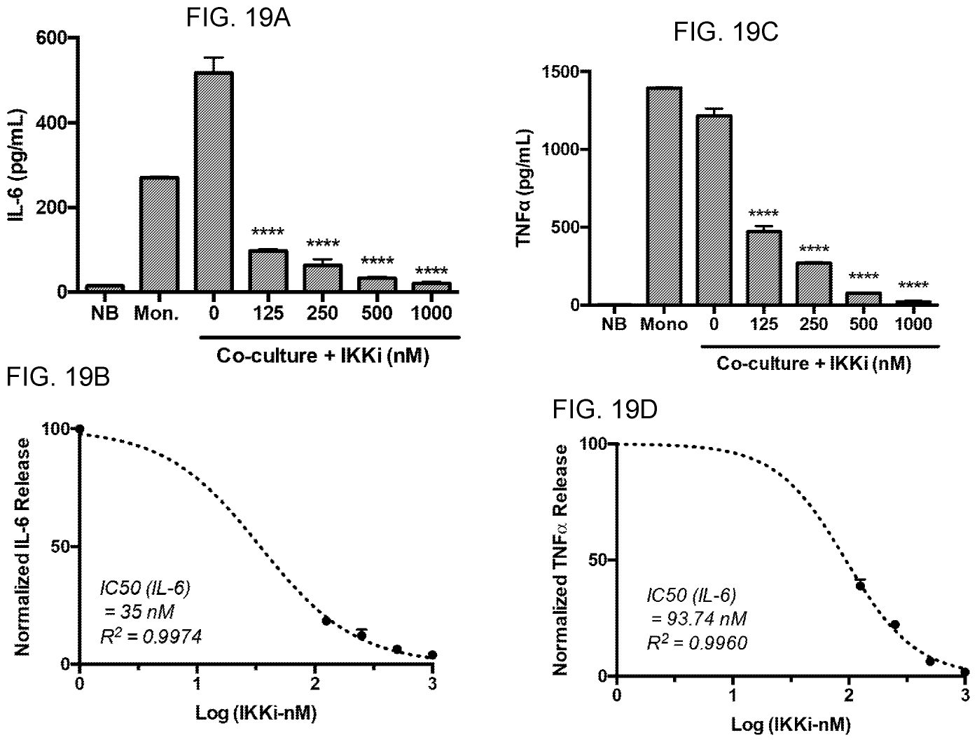

In monocytes and macrophages, the canonical NF-κB signaling pathway is a primary route to induce IL-6 production (17). NB-monocyte co-cultures were treated with IKK inhibitor VII (IKKi), which blocks canonical NF-κB signaling, and observed dose-dependent abrogation of both IL-6 and soluble (s)TNFα production from monocytes (FIG. 12C,D, FIG. 19). These results highlight the importance of canonical NF-κB signaling in production of these cytokines during NB-monocyte co-culture. Given that TNFα is a major activator of the NF-κB pathway, the effect of TNFα neutralization on monocyte activation in the co-culture was then evaluated. Treatment with an anti-TNFα antibody reduced monocyte IL-6 production by 60% (FIG. 12E) and led to a 35% increase in IκBα levels compared to isotype antibody-treated controls (FIG. 12F). IκBα sequesters NF-κB in the cytoplasm, blocking downstream signaling; therefore, IκBα expression relates inversely to NF-κB signaling activity (18). Together, these data demonstrate that NB cells activate NF-κB signaling in monocytes in a contact-dependent manner that leads to downstream IL-6 production and requires TNFα.

### XII. Example 12: Monocyte mTNFα Mediates NF-κB Activation and Pro-tumorigenic Inflammation

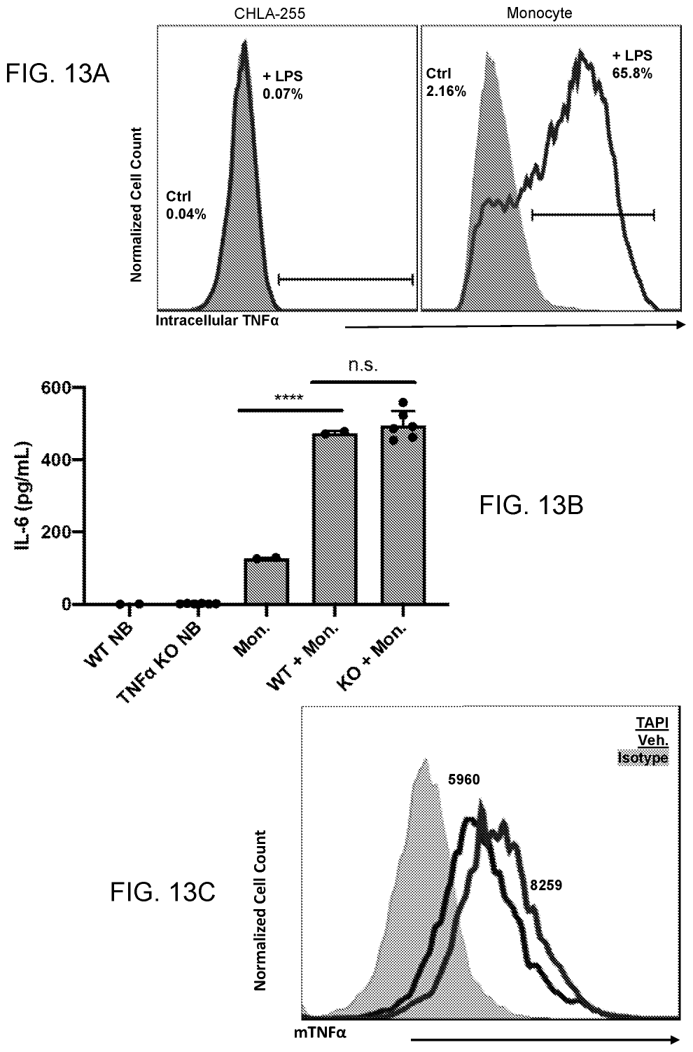

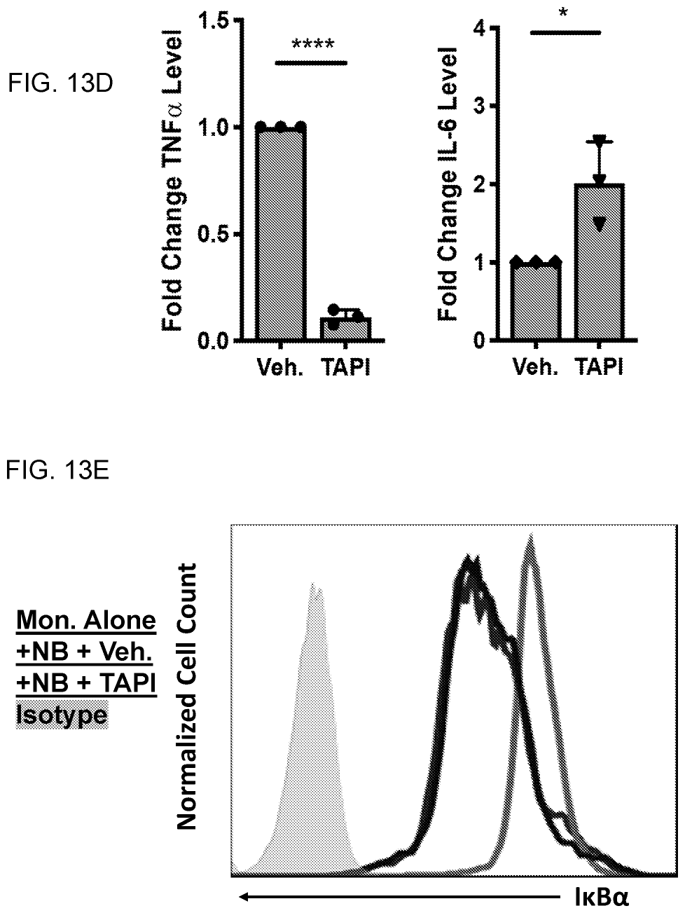

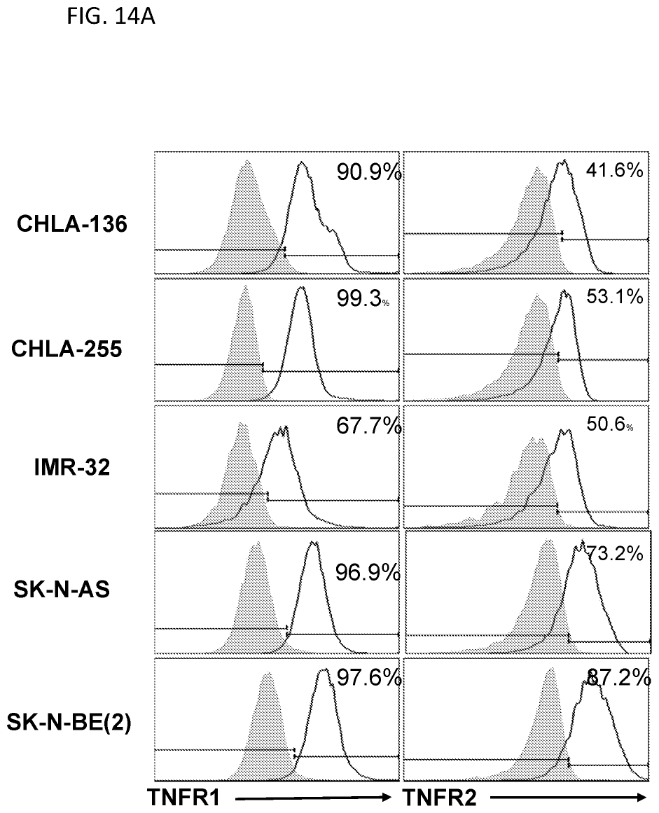

Next, the respective contributions of NB cells and monocytes to the production of TNFα within the co-culture was evaluated. It was found that CHLA-255 cells did not express TNFα, even after stimulation with LPS (<0.1%); conversely, a majority of LPS-stimulated monocytes expressed TNFα (>65% TNFα+ cells) with a low level of basal expression observed (2% TNFα+) (FIG. 13A).

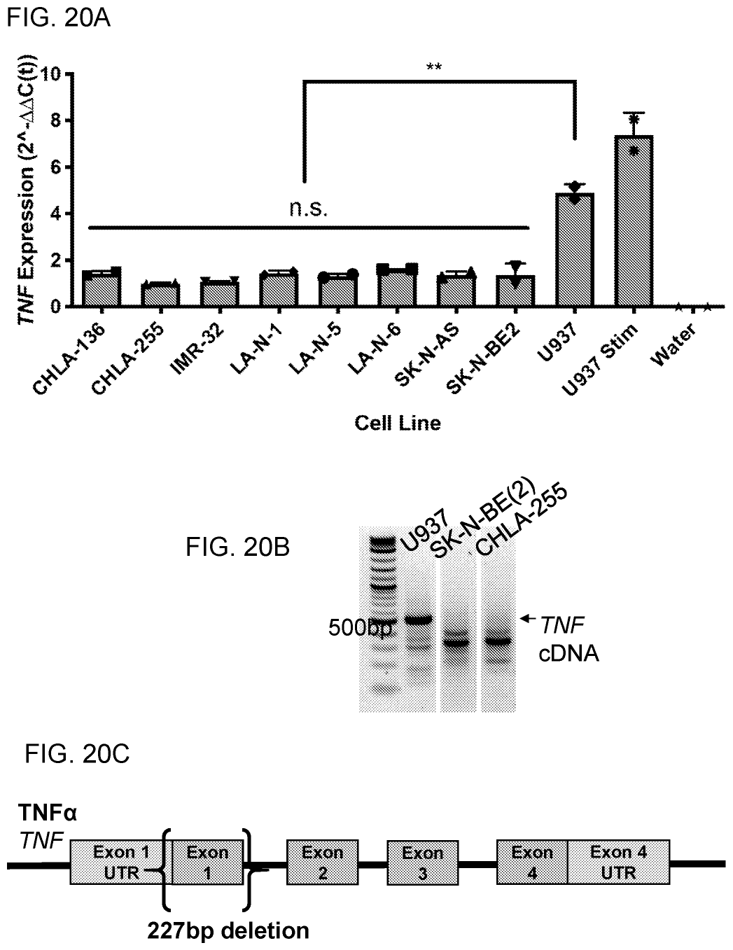

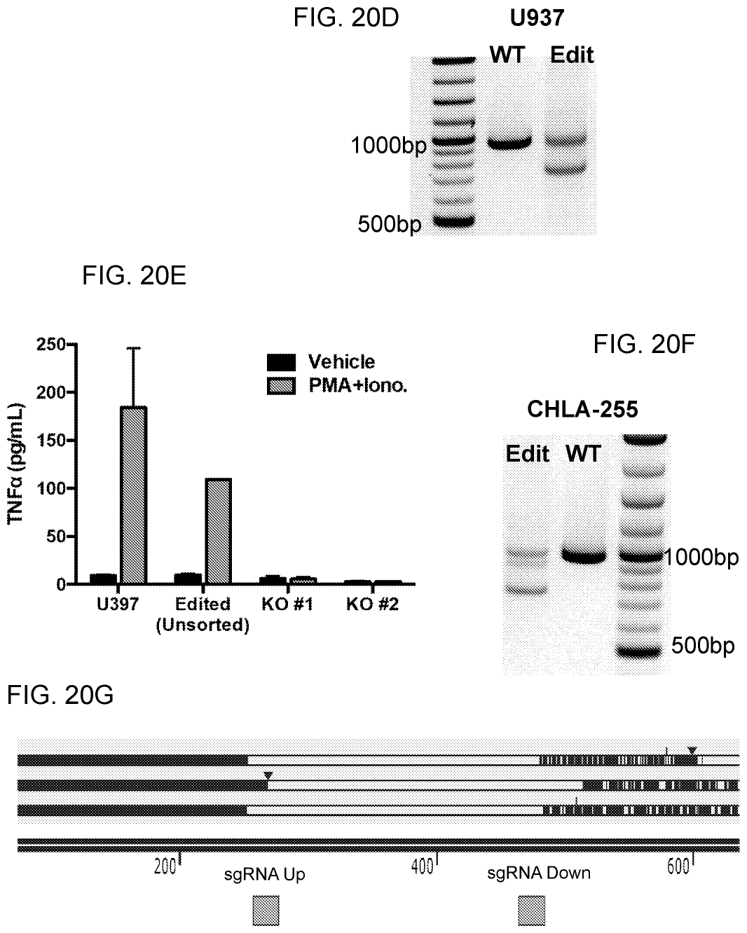

Furthermore, a panel of eight NB cell lines showed no TNF mRNA expression by qRT-PCR (FIG. 20A,B). To verify this, TNFα knock-out (KO) NB were generated using CRISPR/Cas9 (FIG. 20C). To validate the KO system, successful KO of the TNF gene encoding TNFα was generated in monocytic leukemia cell line U937, a standard TNFα positive control, before generating CHLA-255 KO single-cell clones (FIG. 20D-G). Co-culture of monocytes with TNFα KO versus wild-type CHLA-255 yielded similar levels of IL-6 production, indicating that monocytes are the source of TNFα in these co-cultures (FIG. 13B).

TNFα is expressed in two distinct isoforms within cells: as a trimeric transmembrane protein (mTNFα) and as a soluble cytokine (sTNFα) cleaved from mTNFα by TNFα converting enzyme (TACE) (19, 20). These isoforms perform different cellular functions and have previously been reported to modulate differential signaling in the TME (21). To evaluate the contribution of each isoform to NF-κB activation in monocytes, co-cultures were treated with the TACE inhibitor TAPI, which blocks formation of sTNFα by inhibiting cleavage of mTNFα. Treatment with TAPI led to a 40% increase in surface staining for mTNFα, as well as a 90% reduction in sTNFα levels within the NB-monocyte co-culture supernatant (FIG. 13C, D). Despite this reduction in sTNFα, TAPI treatment significantly increased IL-6 levels (P=0.0292) in the co-culture supernatant compared to a vehicle control; additionally, TAPI treatment appeared not to impact NF-κB activation, as treated co-cultures had similar levels of intracellular IκBα as the control group (FIG. 13E, F). Collectively, these findings demonstrate that monocyte-derived mTNFα, but not sTNFα, is sufficient to activate NF-κB signaling in monocytes that leads to downstream production of IL-6 during co-culture with NB cells.

### XIII. Example 13: TNFR1 and TNFR2 Play Distinct Roles in Pro-Tumorigenic Signaling Loop Between NB Cells and Monocytes

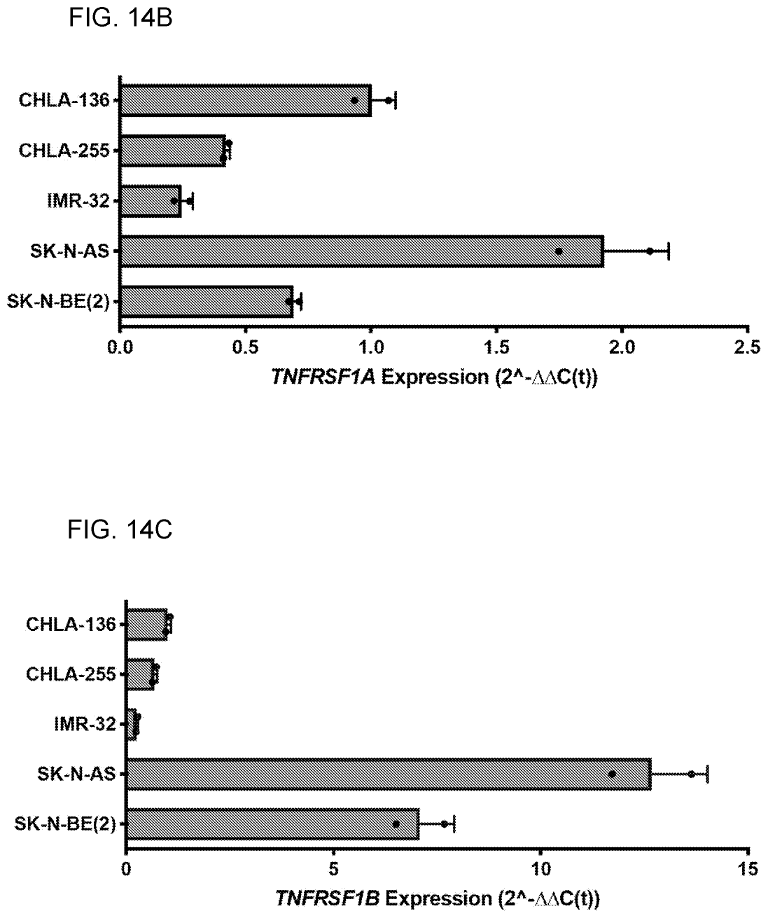

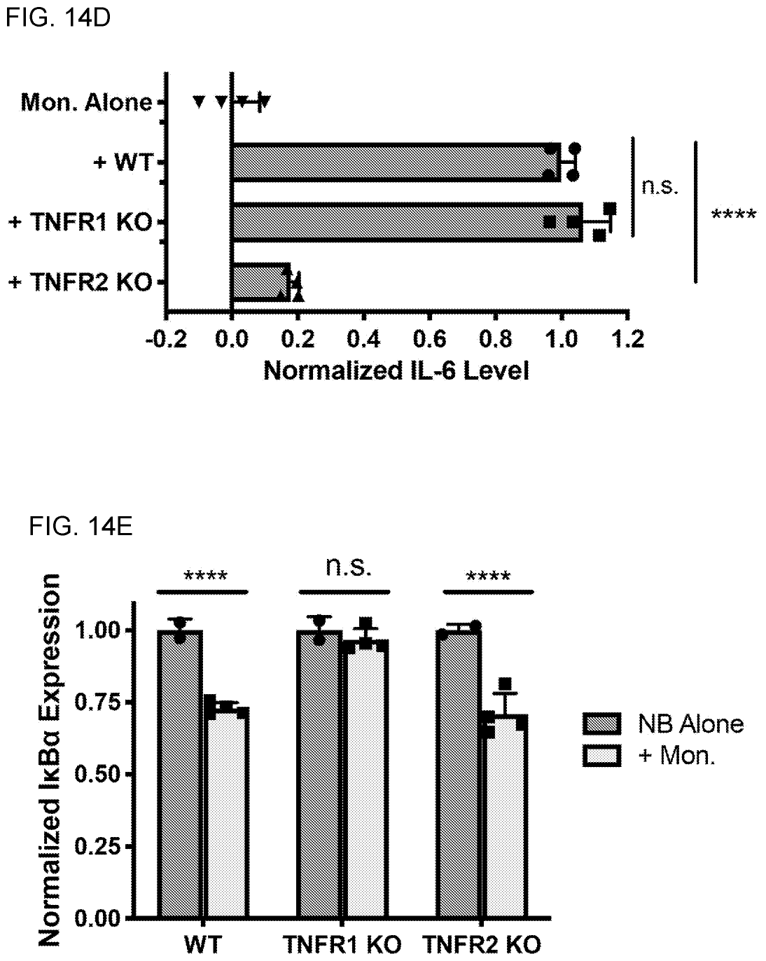

TNFα signals through TNFα receptors 1 and 2 (TNFR1/2), with the secreted form mainly interacting with TNFR1 and the membrane-bound form with TNFR2. TNFR1 and 2 employ different signal transduction mechanisms and both drive survival and death pathways in a cell-specific manner (22). The expression of TNFR1 and 2 in a panel of NB cell lines was evaluated and found that all lines expressed both receptors, with SK-N-AS and SK-N-BE(2) expressing the highest levels (FIG. 14A); surface expression also correlated with TNFRSF1A (encodes TNFR1) and TNFRSF1B (encodes TNFR2) mRNA expression (FIG. 14B,C).

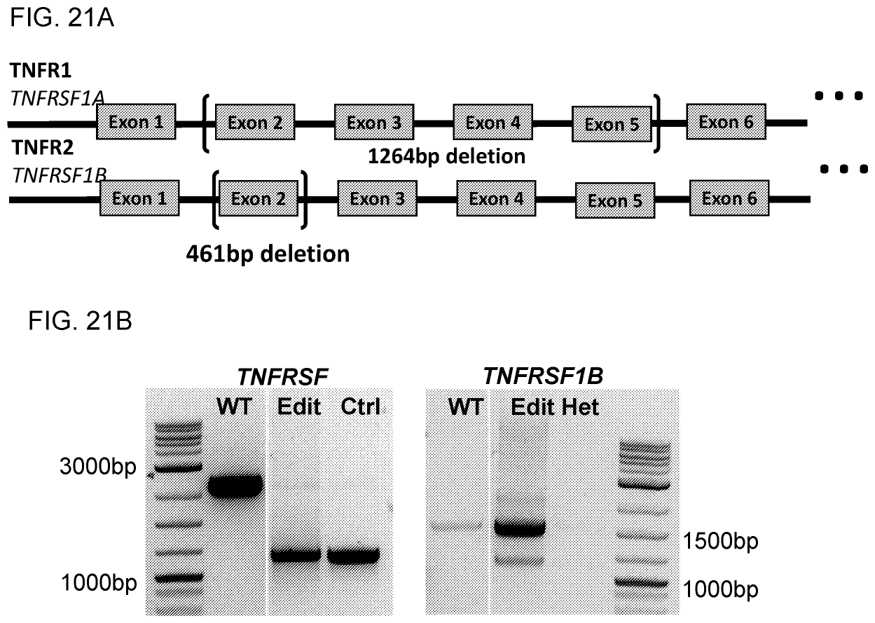

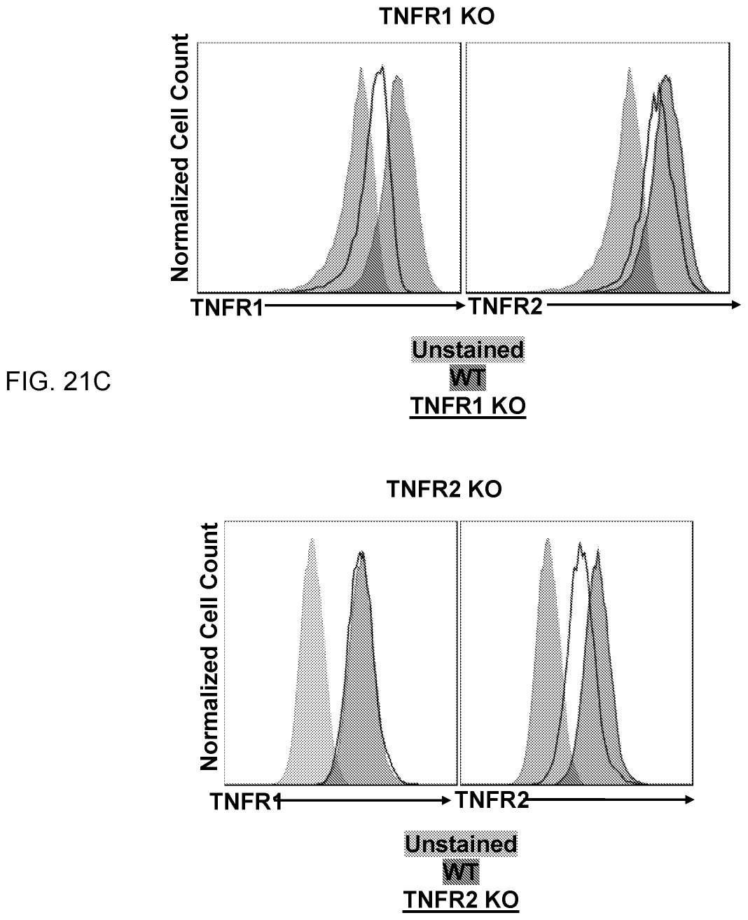

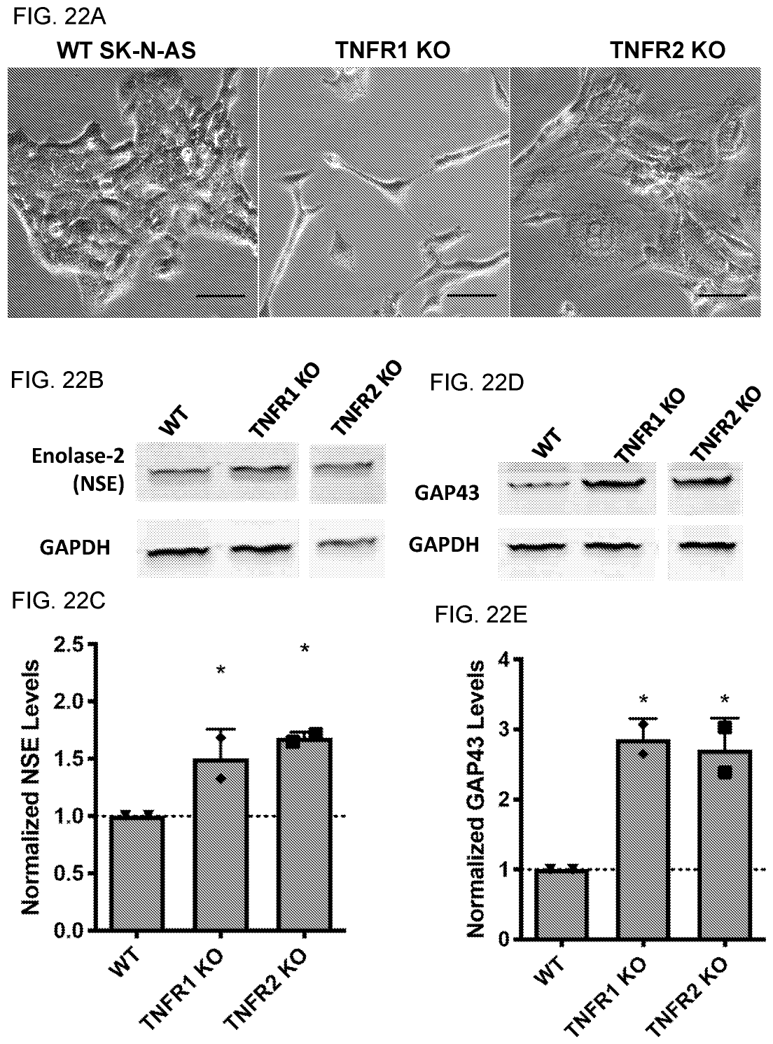

Next, a single-cell KO clones was generated for both TNFR1 (TNFRSF1A KO) and TNFR2 (TNFRSF1B KO) in the SK-N-AS, CHLA-255 (both MYCN-NA), and SK-N-BE(2) (MYCN-A) NB cell lines (FIG. 21A,B). CHLA-255 and SK-N-AS TNFR1/2 KO clones appeared to differentiate before they could be evaluated (FIG. 22A); SK-N-BE(2) TNFR1/2 KO clones showed minimal phenotypic changes (data not shown) but expressed increased levels of neural differentiation markers growth associated protein 43 (GAP43) and enolase-2 (NSE) compared to wild-type cells (FIG. 22B-E). These findings suggest that TNFR1 and TNFR2 may have intrinsic roles in maintaining NB in a less differentiated state.

Of note, a compensatory increase was not observed in the remaining receptor expression (TNFR2 in TNFR1 KO, TNFR1 in TNFR2 KO) for SK-N-BE(2) KO clones (FIG. 21C). Therefore, to determine the role of each TNFR in TNFα signaling within the NB-monocyte co-culture, these SK-N-BE(2) TNFR1/2 KO clones were cultured with freshly isolated monocytes and evaluated IL-6 levels after 24 hours. It was found that only TNFR2 KO in NB impacted monocyte activation, reducing co-culture IL-6 levels by 80% (FIG. 14D). These findings demonstrate that NB TNFR2 is crucial for induction of IL-6 production in monocytes, consistent with the observation that mTNFα mediates this induction on the monocytic side.

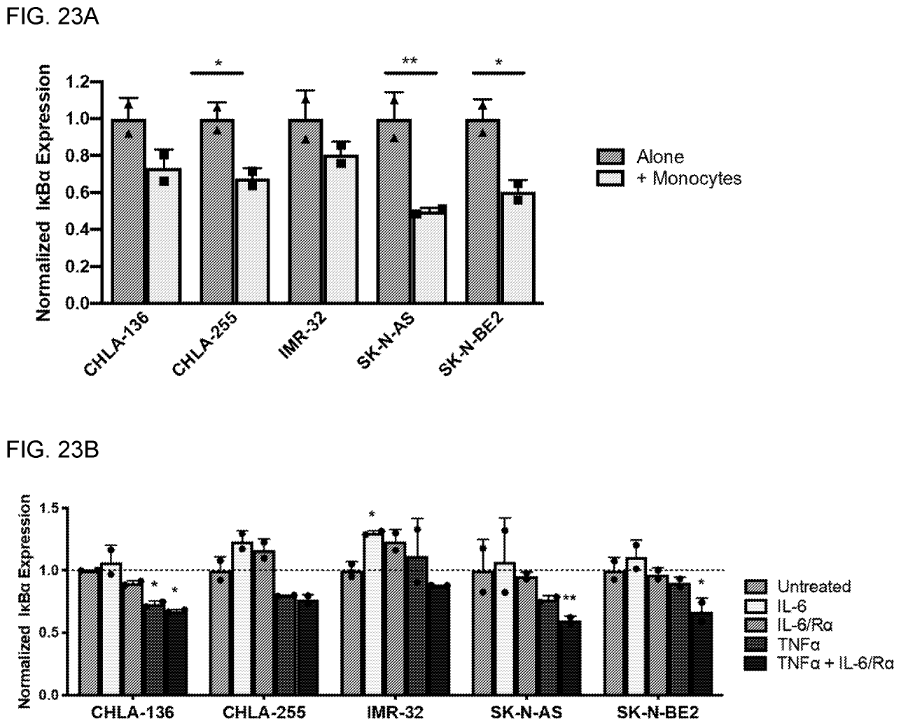

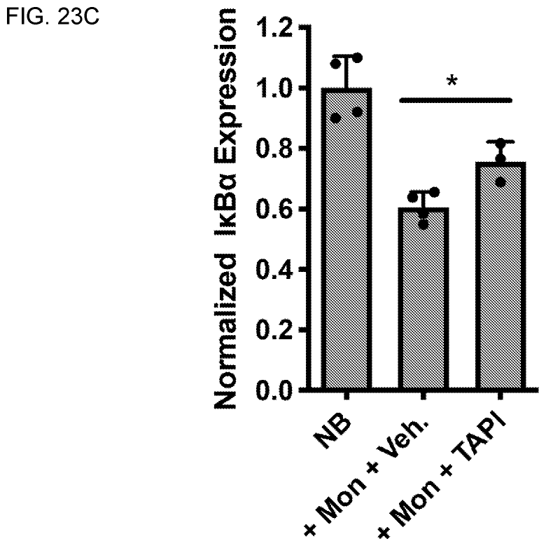

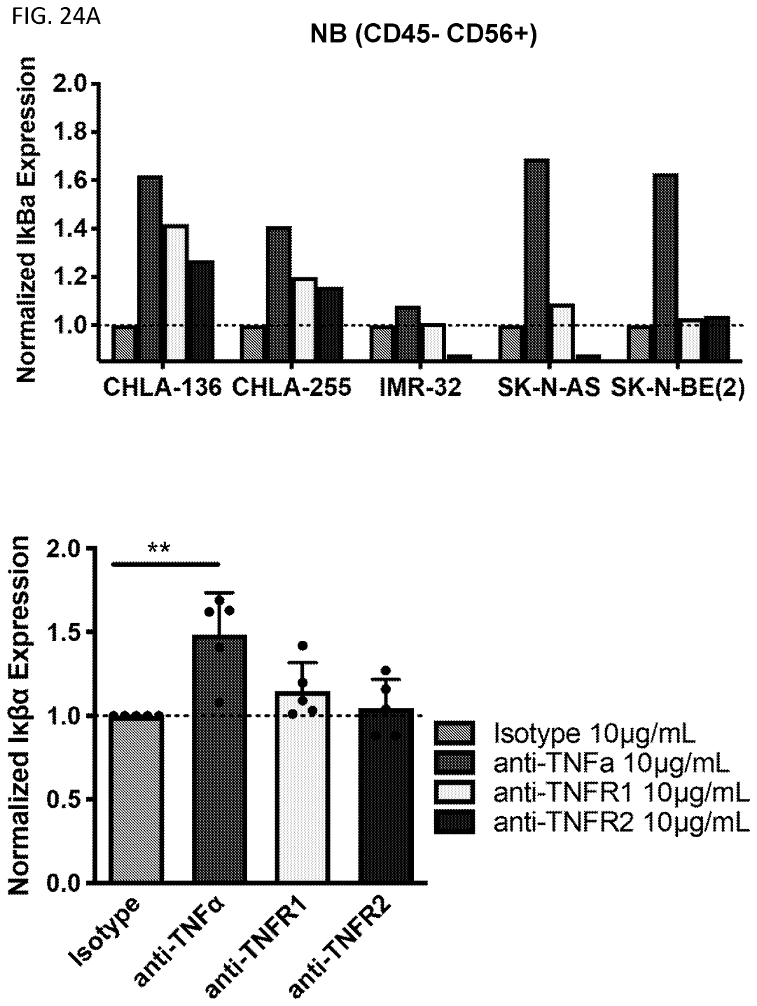

To determine the impact of TNFR1/2 KO on NB itself, IκBα expression levels was measured as a read-out for NF-κB activation following monocyte co-culture. It was found that NB NF-κB activation was only impacted when the gene encoding TNFR1, but not TNFR2, was knocked-out, as shown by a relative increase in IκBα levels following monocyte co-culture compared to the wild-type control (FIG. 14E). To further investigate NB NF-κB signaling, which in some embodiments is found to be more active in NB cell lines with the highest TNFR expression (FIG. 23A), TNFα and/or IL-6 contribution to this activation was explored. In NB cell lines treated with TNFα, IL-6, IL-6 with secreted (s)IL-6Rα, or all three, TNFα led to a drop in IκFα levels compared to untreated controls, which became even more pronounced in combination with IL-6 and sIL-6Rα (FIG. 23B). Having established the importance of TNFR1 and TNFα in NB NF-κB activation, it was determined whether this activation is dependent on a particular TNFα isoform. NB-monocyte co-cultures treated with TAPI to block sTNFα production showed a significant increase in IκBα expression level versus the vehicle control group, indicating that sTNFα activates NF-κB signaling in NB cells (FIG. 23C). Taken together, these results suggest that sTNFα signaling through TNFR1 leads to pro-tumorigenic NF-κB activation in NB co-cultured with monocytes.

### XIV. Example 14: TNFα Neutralization Reduces Tumor-Promoting Inflammation In Vitro

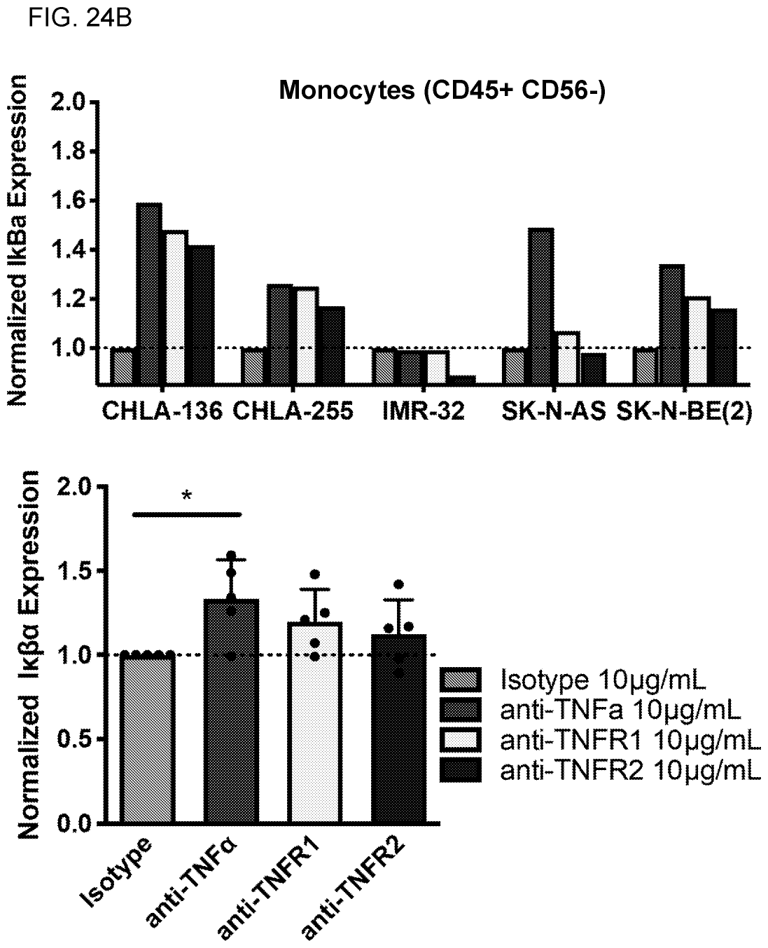

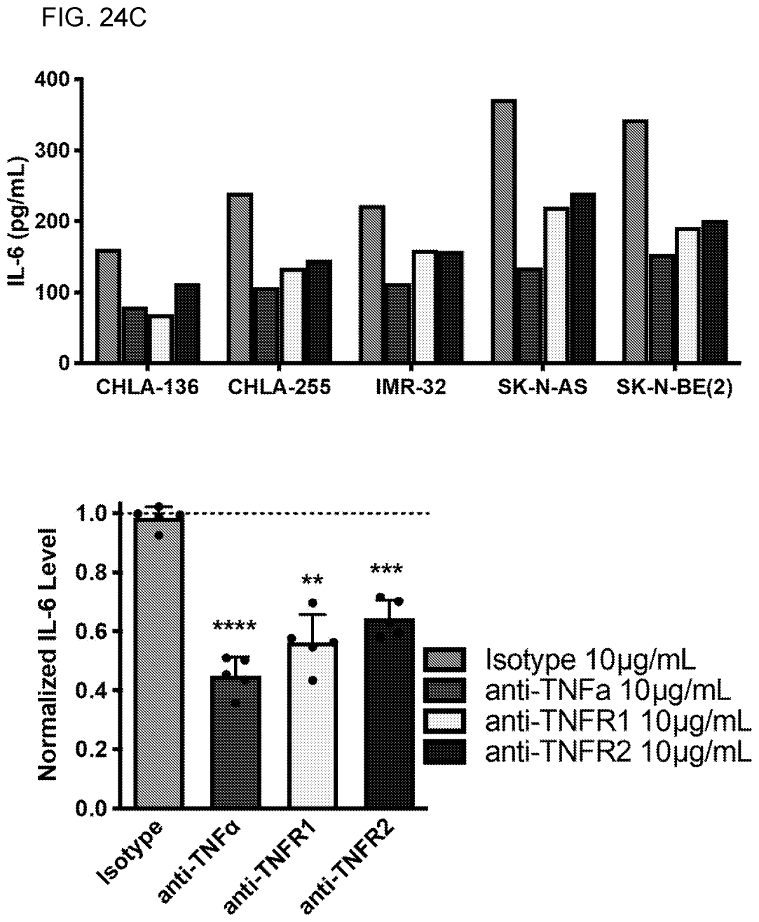

The results presented thus far describe a mechanism in which NB TNFR2 activates reverse signaling of mTNFα on the monocyte surface, leading to activation of NF-κB and production of pro-tumorigenic cytokines including IL-6 and sTNFα. To evaluate the potential therapeutic efficacy of targeting TNFα signaling in the NB TME, a panel of NB cell lines co-cultured with monocytes was treated with research-grade TNFα-, TNFR1-, and TNFR2-neutralizing antibodies and evaluated the effect on NF-κB activation and IL-6 production. When averaged across all cell lines, only the TNFα-neutralizing antibody reduced IκBα degradation in both monocytes and NB (FIG. 24A,B). All antibodies reduced IL-6 levels, but TNFα neutralization led to the greatest reduction in IL-6 levels across all cell lines (range 40-70% reduction) (FIG. 24C).

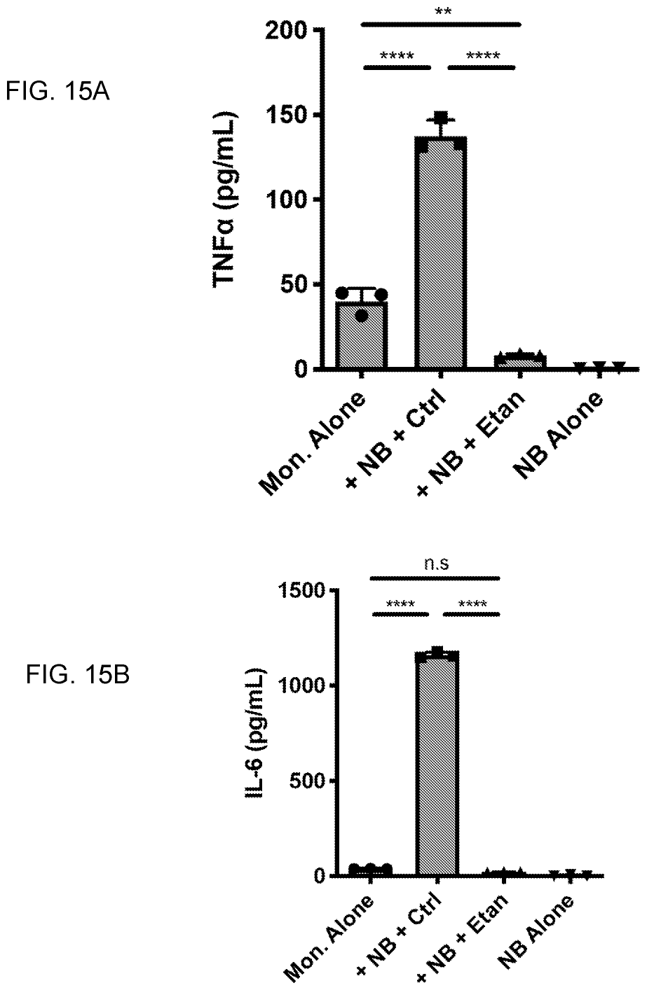

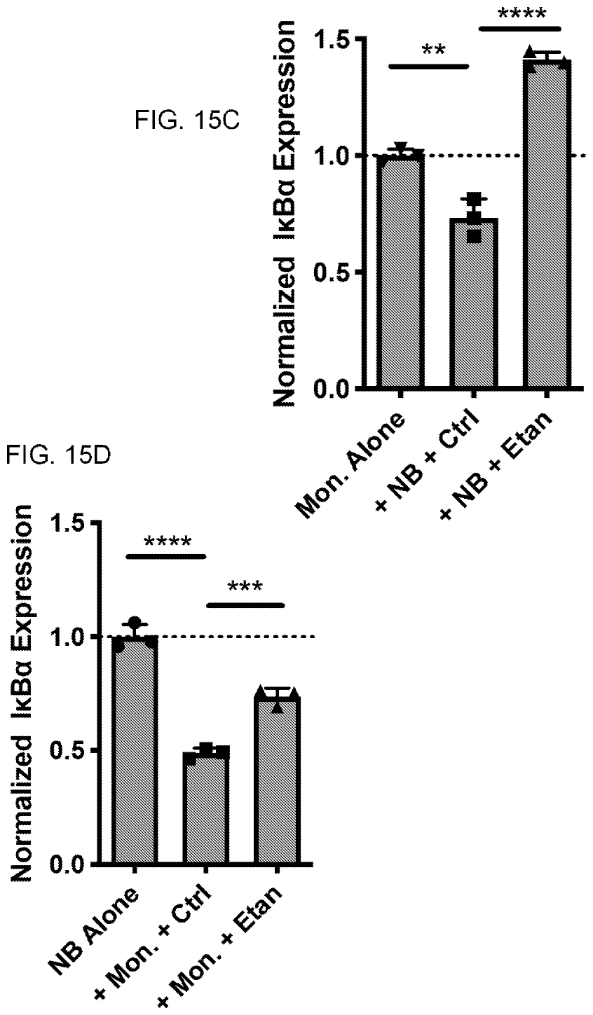

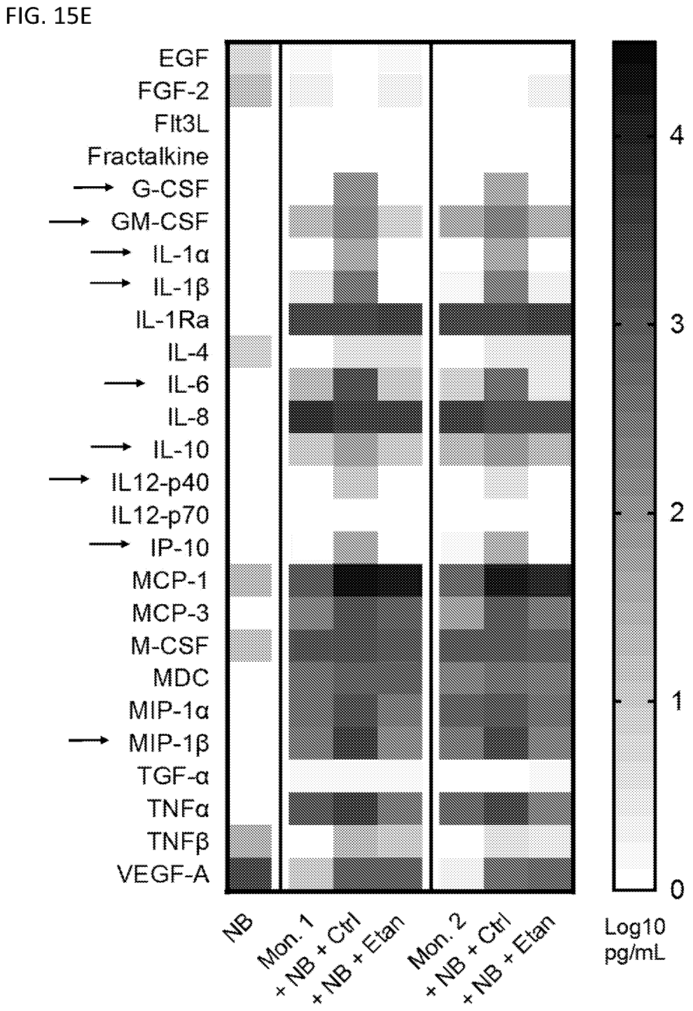

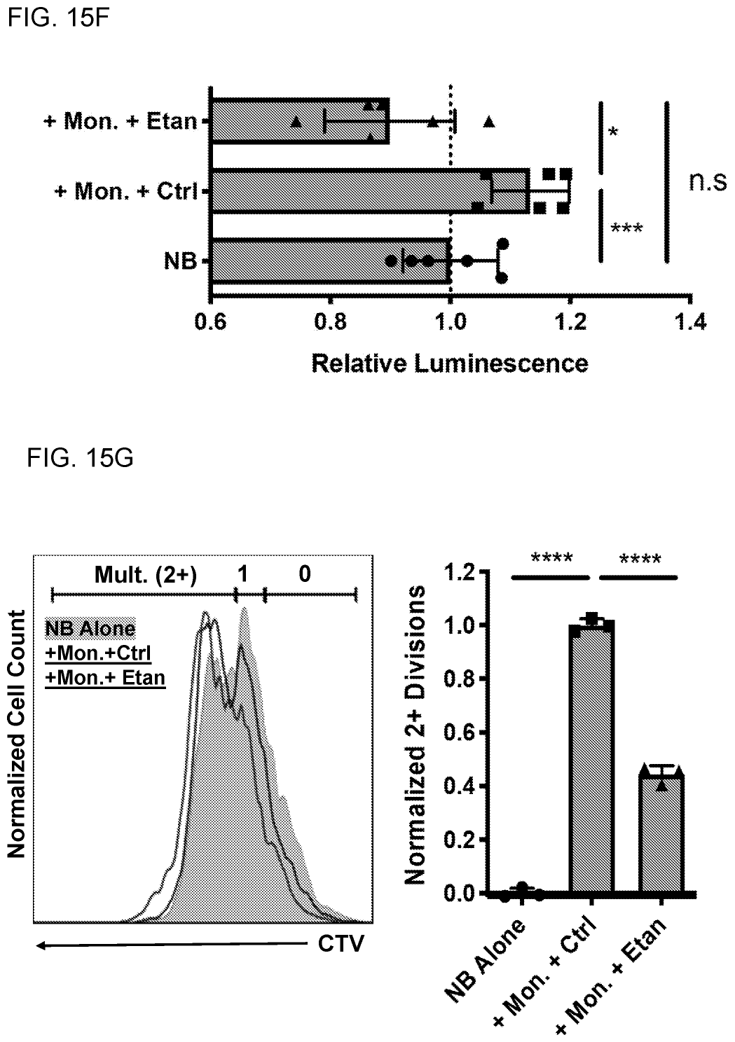

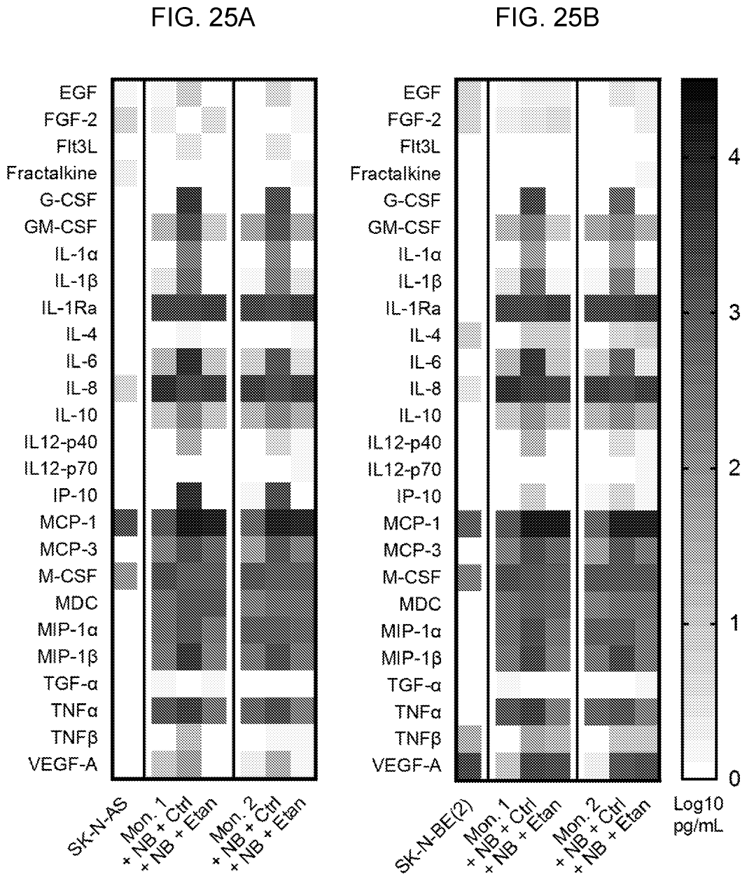

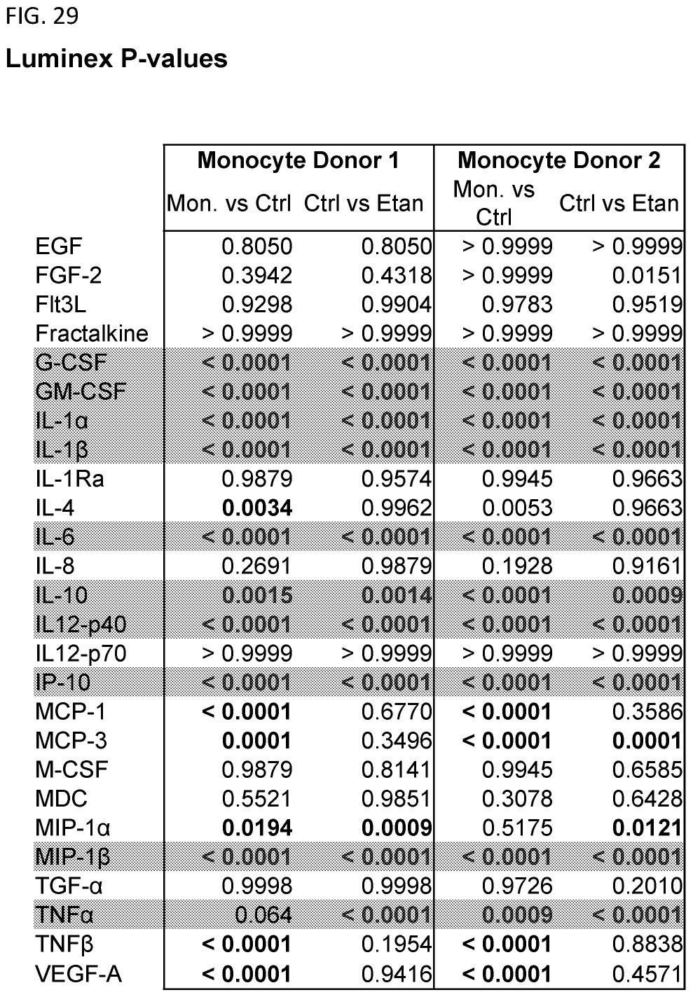

To explore the clinical relevance of this mechanism, FDA-approved etanercept, an Fc-TNFR2 fusion protein that neutralizes signaling by both TNFα isoforms (23) was used. Treatment of NB-monocyte co-cultures with etanercept almost entirely blocked sTNFα and IL-6 production and led to a complete and partial reversal of IκBα degradation in monocytes and NB, respectively (FIG. 15A-D). Next, a multiplex Luminex cytokine array was performed to evaluate global changes in cytokine production in the co-culture following etanercept treatment. Nine cytokines/chemokines (G-CSF, GM-CSF, IL-1α, IL-1β, IL-6, IL-10, IL-12-p40, IP-10, MIP-1β) were found to be induced or significantly upregulated in CHLA-255-monocyte co-cultures compared to NB or monocytes alone. Etanercept treatment completely reversed the induction/upregulation of all nine cytokines/chemokines (FIG. 15E, FIG. 29). These results were consistent across multiple monocyte donors co-cultured with additional NB cell lines (SK-N-AS and SK-N-BE(2), FIG. 25A,B).

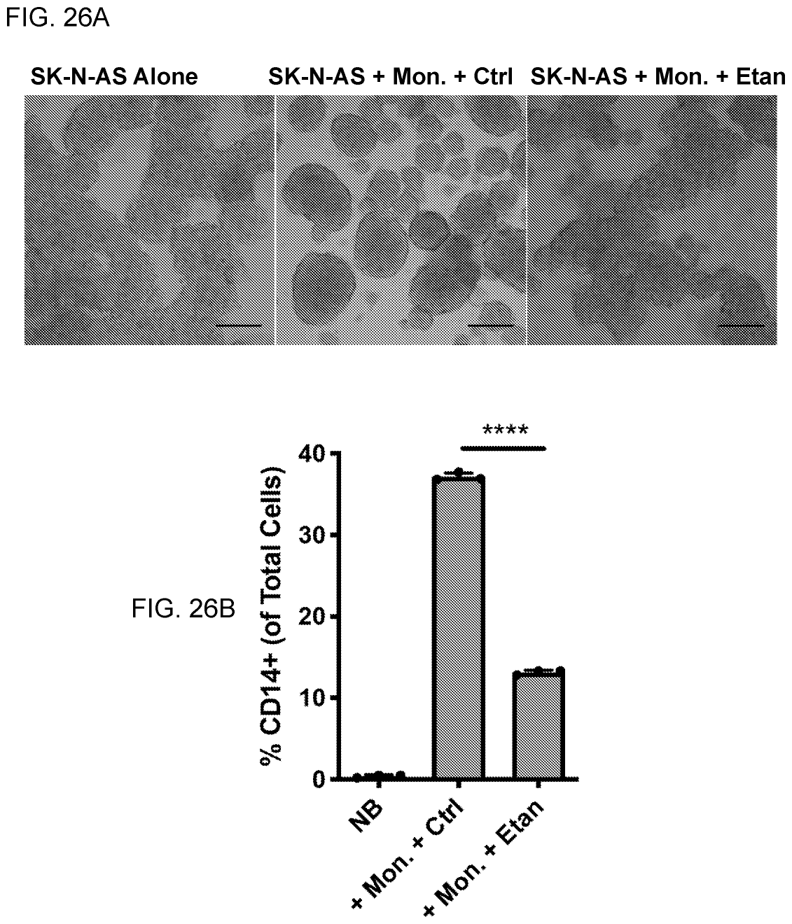

Notably, etanercept treatment also led to dramatic macroscopic changes in NB-monocyte co-cultures, blocking the ability of NB/monocytes to associate into tumorsphere clusters that otherwise form readily in low attachment plates (FIG. 26A). Further, when monocytes were seeded over an adherent NB monolayer, treatment with etanercept decreased the adherence of CD14+monocytes to the NB monolayer (FIG. 26B). This provides additional evidence that TNFα mediates a contact-dependent interaction between NB and monocytes.

To more closely examine the impact of etanercept on NB growth and viability, firefly luciferase-labeled CHLA-255 (CHLA-255-luc) were co-cultured with monocytes and treated with etanercept, using NB luminescence as a readout for growth/viability relative to a NB-only baseline control. Co-culture of NB cells with monocytes in the presence of control IgG monoclonal antibody resulted in a significant increase in luminescence intensity compared to NB cells cultured alone (P=0.0008); addition of etanercept to the co-culture completely abrogated the effect of monocytes on NB cells (P=0.0429, FIG. 15F). To examine whether this was due to reduced proliferation of NB cells, CHLA-255 cells were pulsed with CellTrace Violet (CTV) and co-cultured with monocytes in the presence of etanercept. Under these conditions, it was found that monocytes induced a significant increase in the percentage of NB cells undergoing multiple divisions (47% to 68%, P<0.0001), which etanercept treatment then reduced by 56% (P<0.0001)(FIG. 15G).

These results indicate that etanercept effectively disrupts the mTNFα/TNFR2 signaling axis between NB cells and monocytes, preventing NF-κB activation in both cell types. This reduces the production of pro-tumorigenic cytokines by monocytes, ultimately resulting in decreased proliferation of NB cells in vitro.

### XV. Example 15: TNFα Neutralization Reduces Tumor Growth and Alters the TME

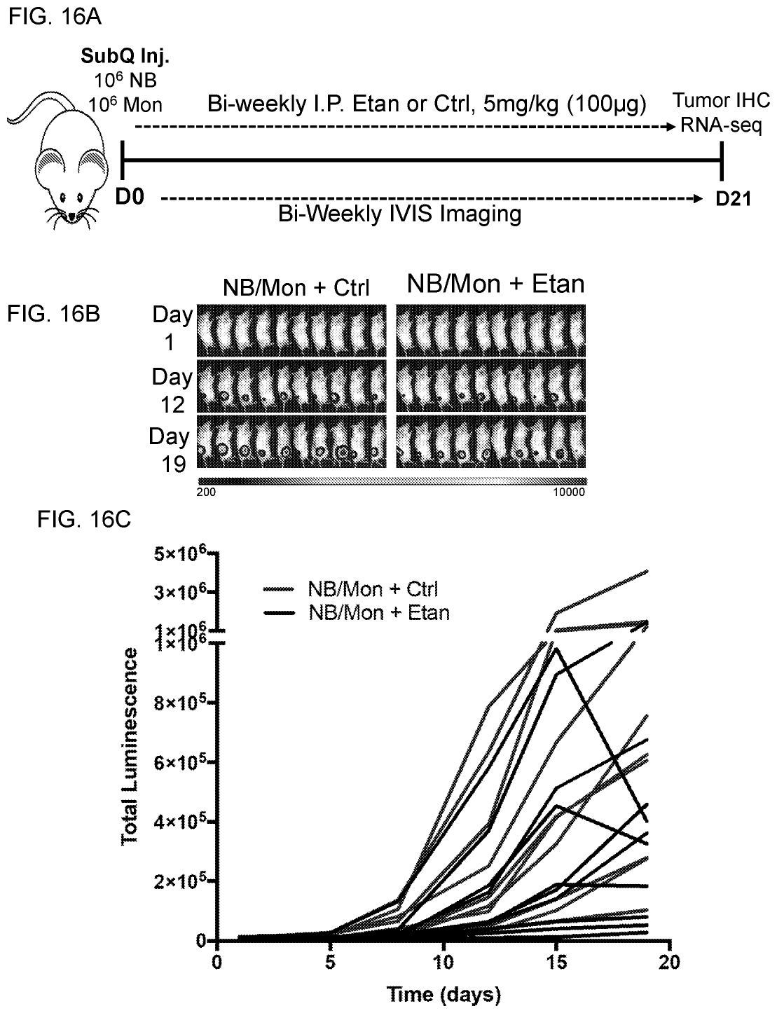

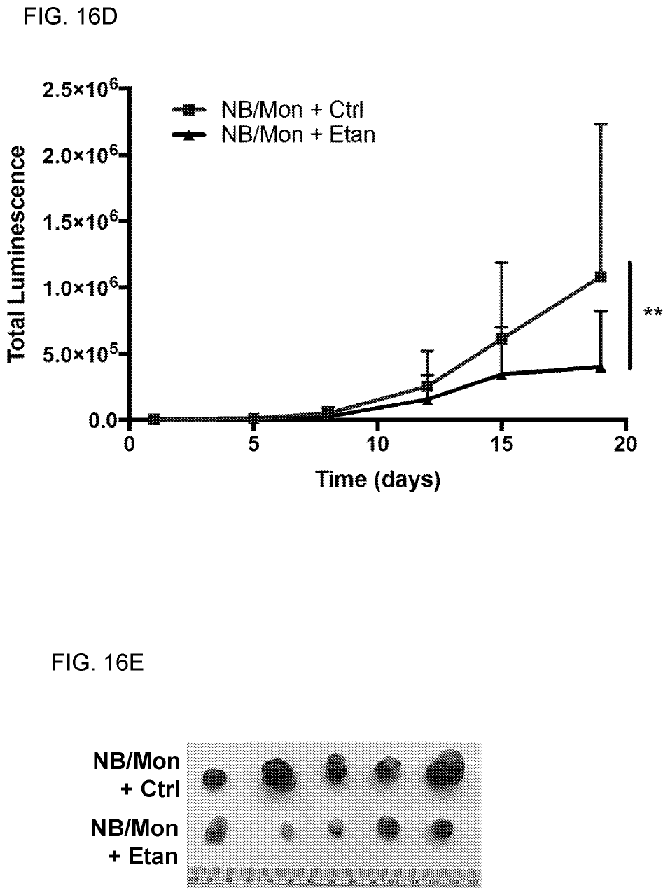

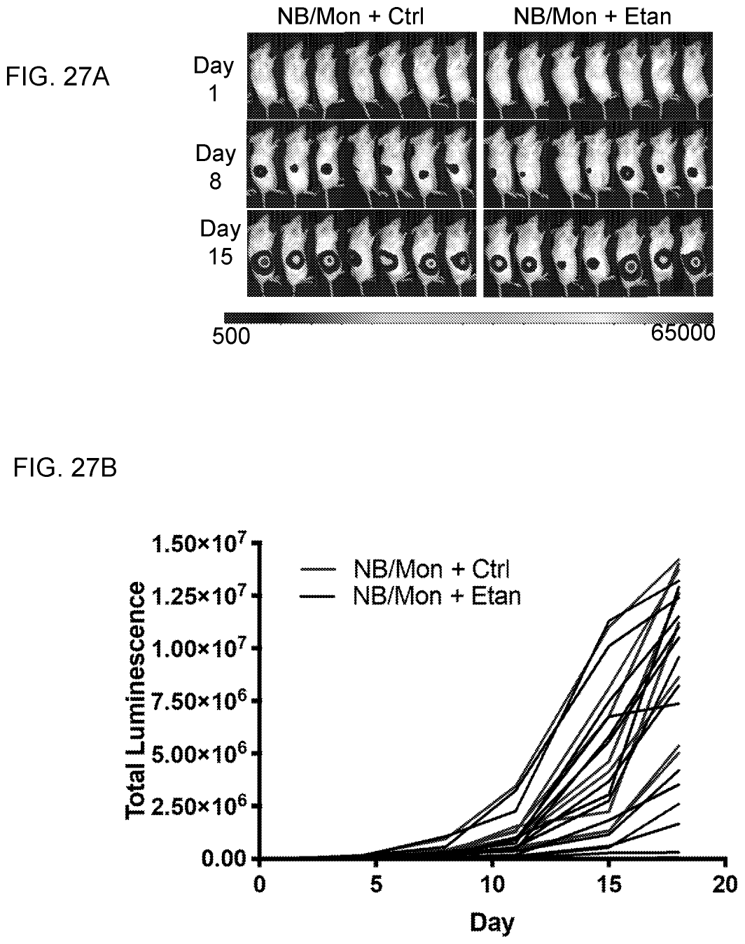

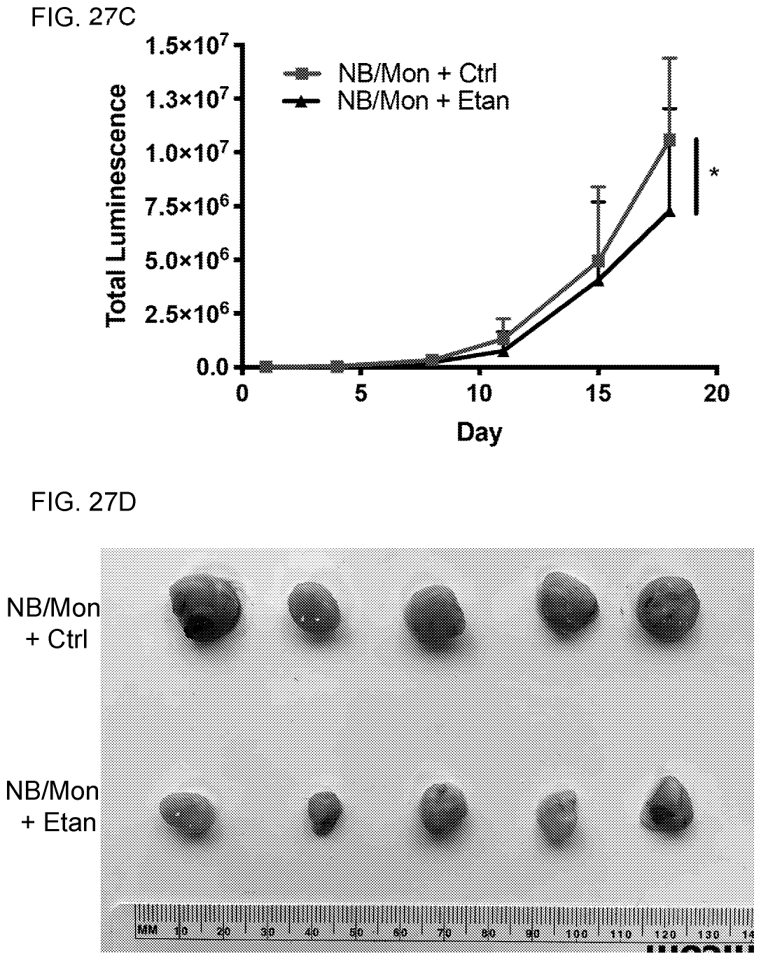

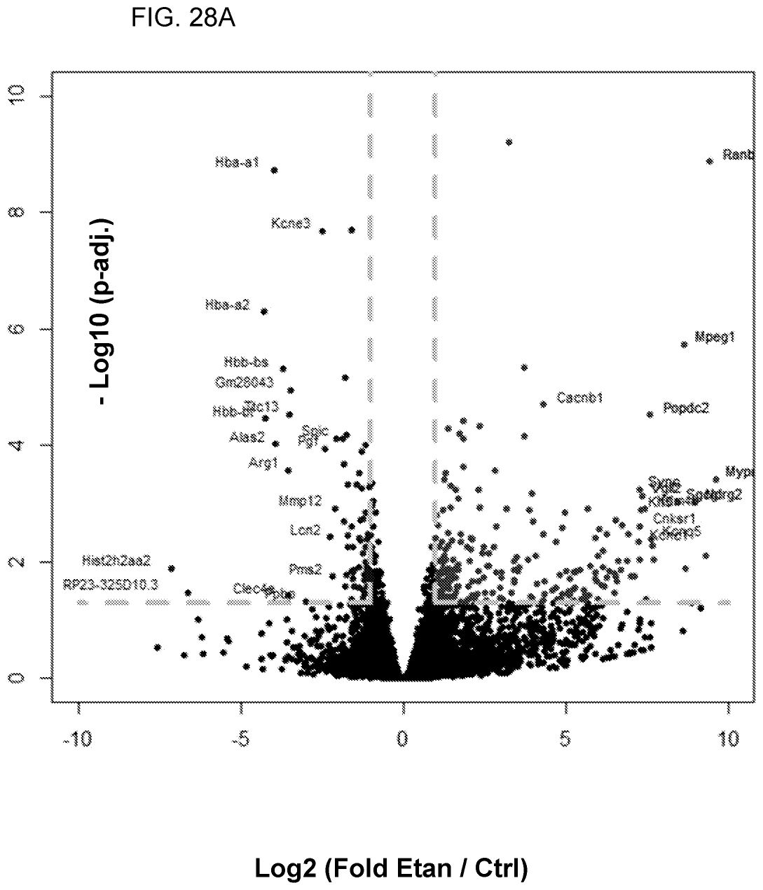

To evaluate the impact of etanercept in vivo, CHLA-255-luc and monocytes embedded in Matrigel were engrafted into the subcutaneous flanks of NSG mice and treated bi-weekly with etanercept or IgG vehicle control (FIG. 16A). Using luminescence readings to track tumor growth (FIG. 16B,C), it was found that etanercept treatment reduced tumor growth by 65% compared to control (P=0.0024)(FIG. 16D,E). Similar results were observed in mice engrafted with SK-N-AS-luc/monocyte xenografts, where etanercept treatment reduced tumor growth by 30% (P=0.0104) (FIG. 27). Importantly, treated tumors appeared to lack blood vessel development and were confined to the superficial subcutaneous tissue of the flank, while control tumors appeared highly angiogenic and invaded into the underlying paraspinal muscles (data not shown).

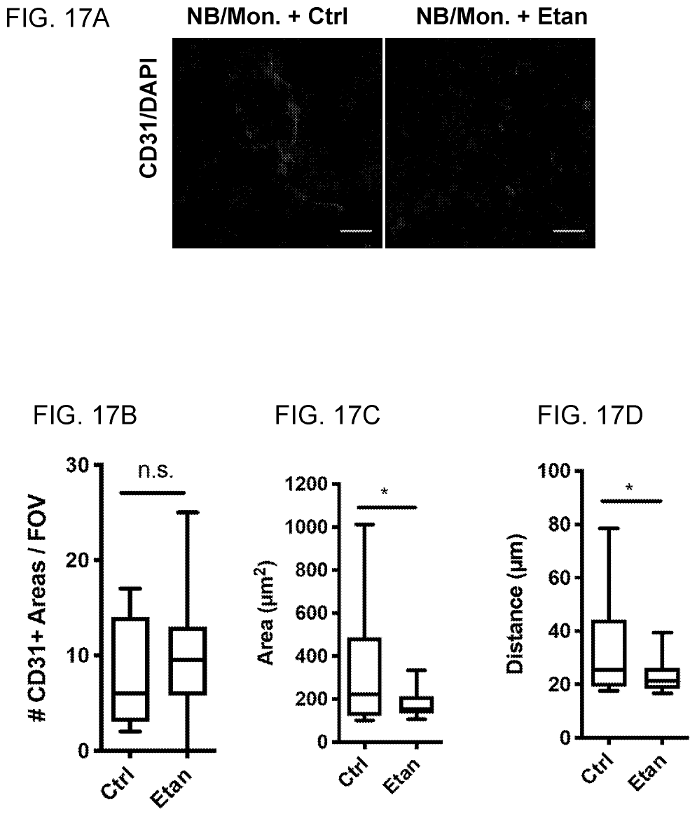

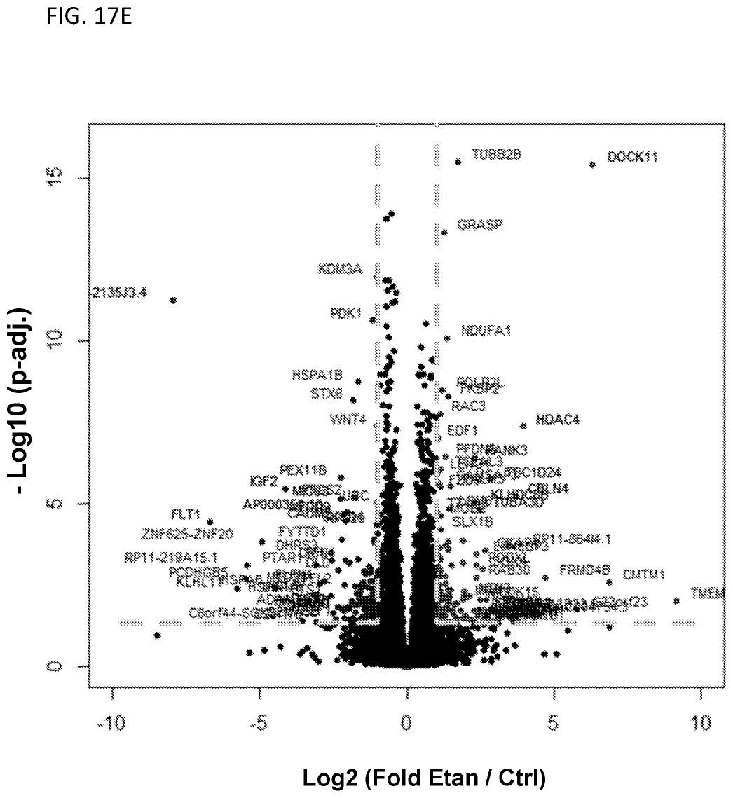

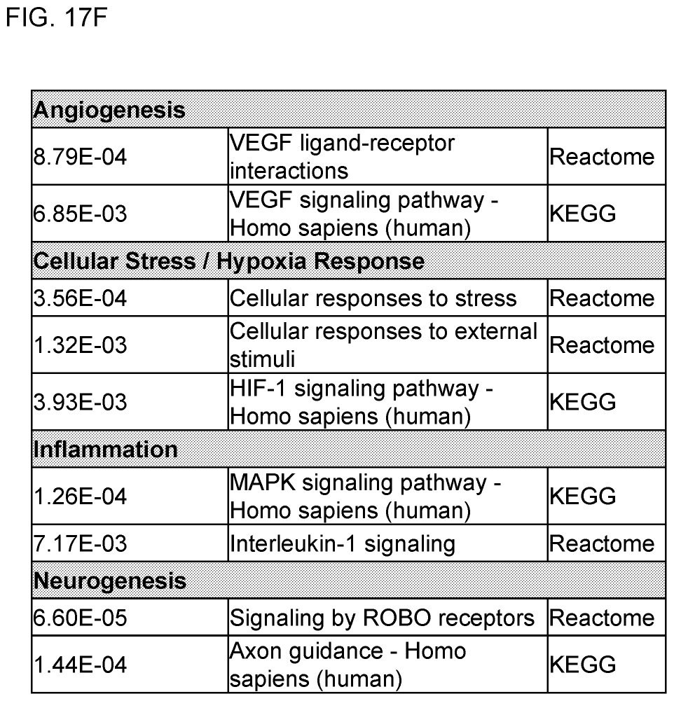

To quantitatively assess angiogenesis, expression of murine CD31, a marker commonly used to identify endothelial cells, was evaluated within the tumor parenchyma. Immunofluorescent staining for CD31 in sectioned tumor tissues showed the formation of complex vasculature in IgG control tumors, while etanercept-treated tumors failed to form distinct vessels despite the presence of CD31+ cells (FIG. 17A). Quantification of these images revealed a similar number of CD31+ regions per field of view (FOV)(FIG. 17B). However, in etanercept-treated tumors, the average area of CD31+ regions per FOV was significantly smaller (P=0.0068) and vessel length as estimated by the maximum linear distance across CD31+ regions was significantly shorter (P=0.0191) than in control tumors (FIG. 17C,D). Overall, these results validate the macroscopic observation that etanercept treatment reduces tumor angiogenesis.

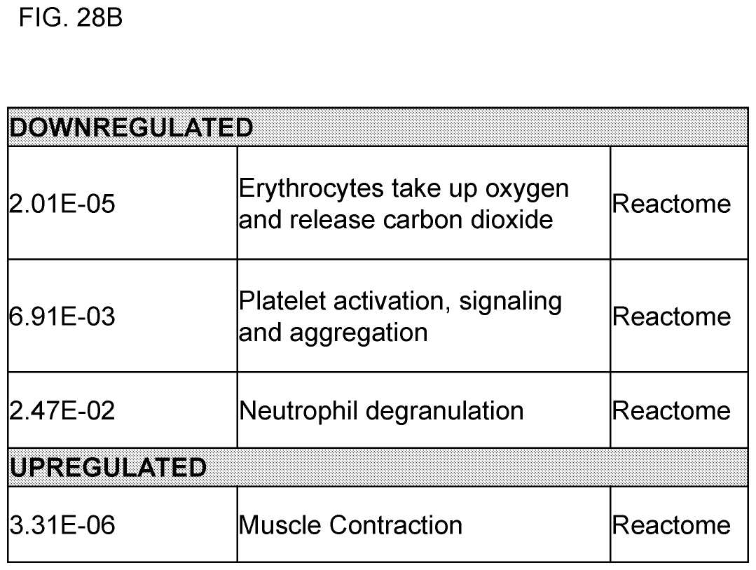

Next, eukaryotic mRNA-seq was performed on representative etanercept-treated and control tumors shown in FIG. 16E to evaluate global changes in NB tumor cell gene expression related to etanercept treatment. On average, 93% of obtained reads mapped to the human genome, 6% mapped to the mouse genome, and 1% were unmappable and subsequently excluded. Within NB transcripts (uniquely human), 138 genes were found that were upregulated and 117 genes that were downregulated in etanercept-treated tumors compared to control tumors (FIG. 17E). Over-representation gene set analysis revealed a decrease in expression of genes associated with angiogenesis (VEGF signaling pathway, P=6.85×10-3), hypoxia and stress responses (cellular responses to stress, P=3.56×10-4; HIF-1 signaling pathway, P=3.93×10-3), MAPK signaling (P=1.26×10-4), and neurogenesis (ROBO receptor signaling, P=6.60×10-5; axon guidance, P=1.44×10-4) in etanercept-treated tumors versus control (FIG. 17F). Further, while sequencing reads from mouse stroma reduced overall statistical power, differential gene expression analysis was able to show that etanercept-treated tumors also have reduced infiltration of erythrocytes, platelets, and neutrophils (FIG. 28). In all, these results are consistent with the observation that etanercept demonstrates potent antitumor activity in vivo by blocking tumor angiogenesis and inhibiting pro-survival signaling in NB cells.

### XVI. Example 16: TNF Signaling is Elevated in MYCN-Non-Amplified NB and Correlates with Poor Outcome

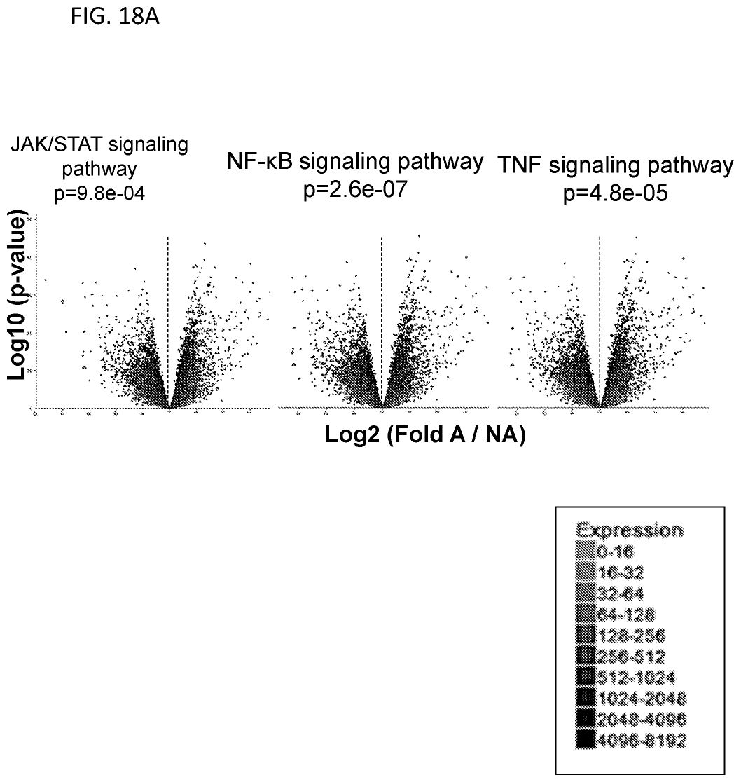

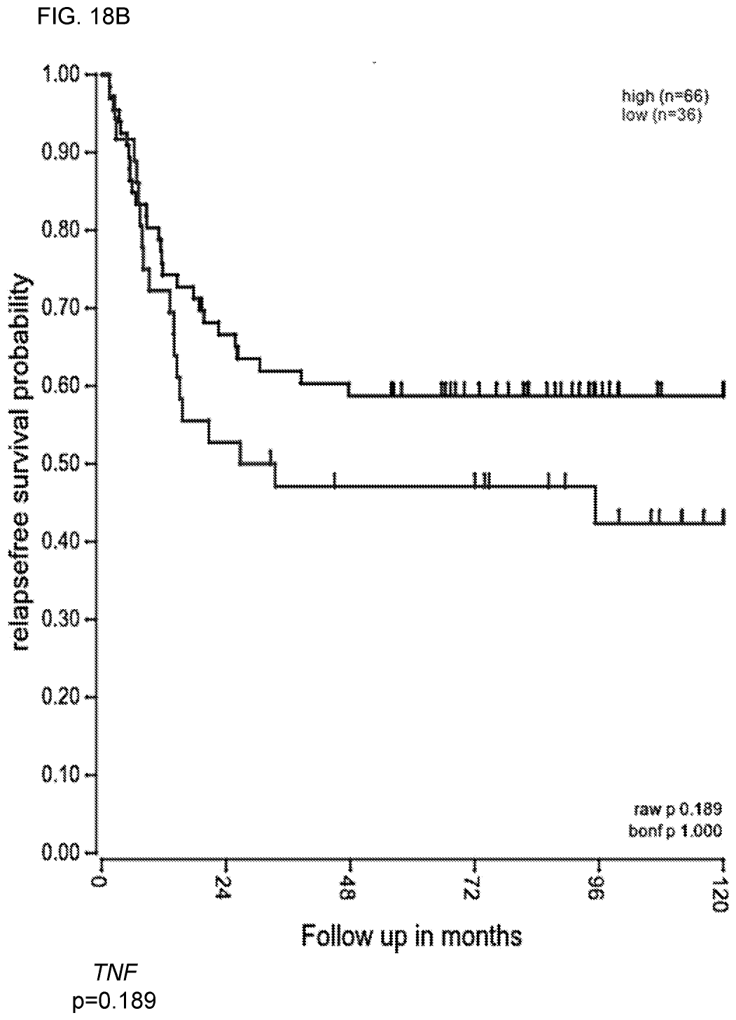

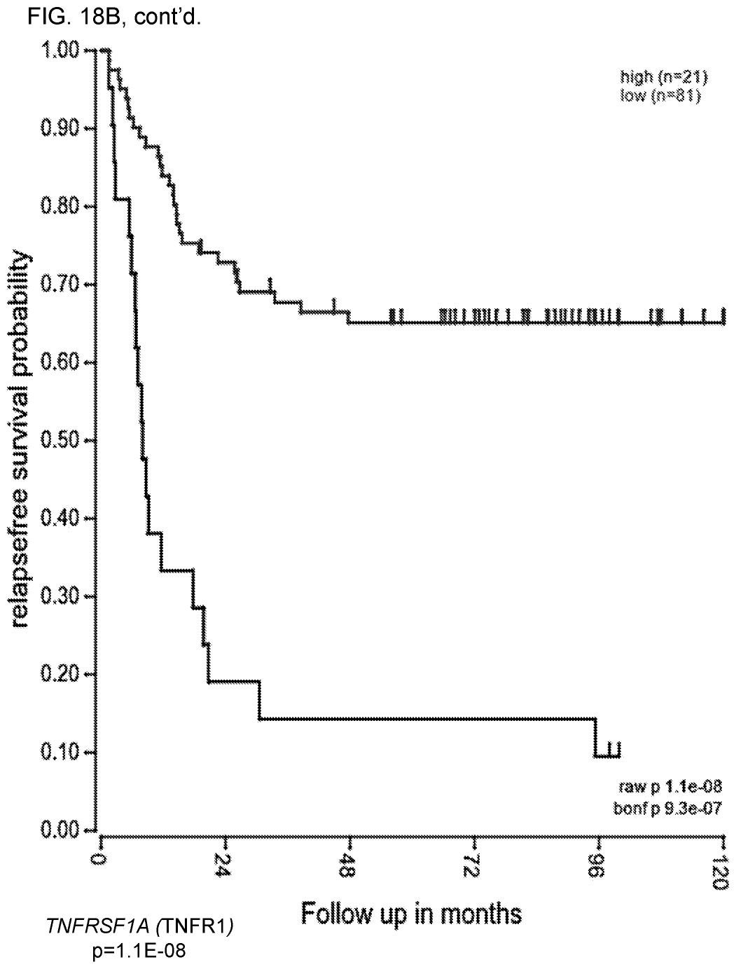

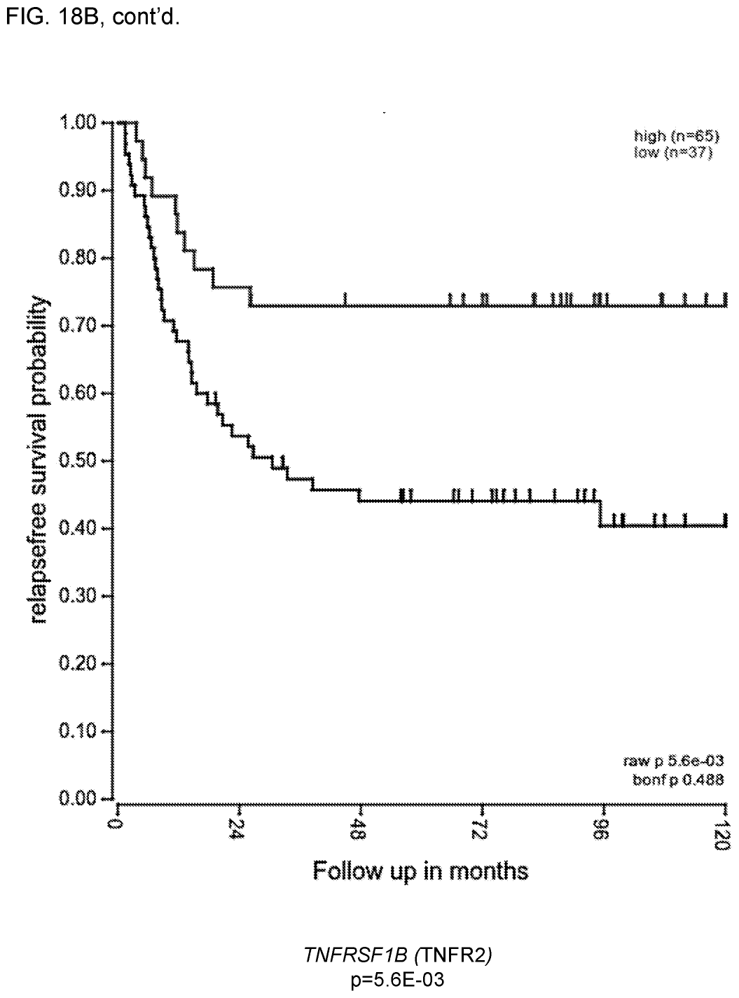

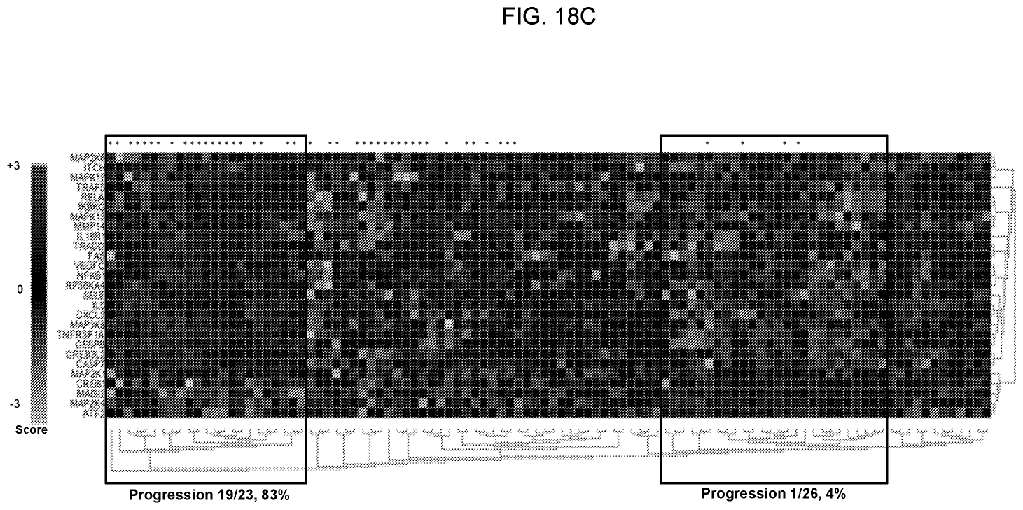

Given the importance of TNFα in the pro-tumorigenic interaction of NB and monocytes, it was asked whether the expression of genes that mediate TNFα signaling has prognostic significance. Using datasets from the R2 Genomics Analysis & Visualization platform (http://r2.amc.nl), first the differential expression of inflammatory signaling pathways was evaluated in MYCN-amplified versus non-amplified stage IV NB tumors. It was found that JAK/STAT, NF-κB, and TNFα signaling pathways were all highly enriched in MYCN-non-amplified NB (P<0.0001)(FIG. 18A). Using Kaplan Meier analysis, it was found that high expression of TNFRSF1A (TNFR1) and TNFRSF1B (TNFR2) correlated with reduced relapse-free survival (P=1.1×10⁻⁸ and P=5.6×10^{'3}, n=102) in patients with stage IV MYCN-non-amplified NB (FIG. 18B). Unsupervised clustering of gene expression was used to evaluate differential expression of genes in the KEGG “TNF_Signaling_Pathway,” comparing MYCN-non-amplified NB patients that progressed with those who did not. Two distinct gene expression clusters correlating significantly with progression-free survival (P=8.0×10⁻³) were found: patients with elevated NF-κB signaling (TNFRSF1A, NFKB1, downstream cytokines, etc.) that had high frequency of progression (19/23, 83%), and patients with low NF-κB signaling that had low frequency of progression (1/26, 4%)(FIG. 18C). Overall, these results reinforce the clinical relevance of TNFα signaling, in particular for patients with MYCN-non-amplified NB.

### XVII. Example 17: Examples of Methods Used in Certain Embodiments

Cell Culture

SK-N-AS, SK-N-BE2, and IMR-32 cells were purchased from ATCC, while CHLA-136, CHLA-255, LA-N-1, LA-N-5, and LA-N-6 were established and maintained as previously described (47). All cell lines were maintained in Iscove Modified Dulbecco Medium (IMDM) supplemented with 20% heat-inactivated FBS (Gibco, Invitrogen) and 2 mM GlutaMax (Gibco, Invitrogen) without antibiotics. Cell lines were routinely checked for mycoplasma contamination (Lonza MycoAlert) every two months.

Plasmids & Retrovirus

pLXIN-Luc was a gift from Alice Wong (Addgene plasmid #60683). To produce retroviral supernatants, 293T cells were co-transfected with three plasmids (Peg-Pam-e encoding gag-pol, DRF encoding the RDF114 viral envelope, and pLXIN-luc containing the luciferase retroviral construct), using the GeneJuice reagent (EMD Millipore Sigma). Viral supernatants were collected 48 hours later and used to transduce SK-N-AS, SK-N-BE2, and CHLA-255 with Polybrene (EMD Millipore Sigma). Transduced cell lines were selected with G418 (EMD Millipore Sigma) for seven days, generating CHLA-255-luc, SK-N-AS-luc, and SK-N-BE2-luc lines.

Monocyte Isolation

PBMCs were isolated by Ficoll-Paque (GE Healthcare) density centrifugation from buffy coats purchased from Gulf Coast Regional Blood Center. Monocytes were isolated by negative selection using the Pan Monocyte Isolation Kit, human (Miltneyi Biotec) according to the manufacturer's guidelines. Monocyte purity was assessed by surface staining for CD14 and CD33. To reduce baseline monocyte activation and cytokine production, purified monocytes were cultured in complete monocyte medium (IMDM with 10% heat inactivated, dialyzed FBS (Gibco, Invitrogen) with 2 mM GlutaMax) in ultra-low attachment (ULA) tissue culture plates (Corning), unless otherwise specified.

Co-Culture Experiments and Treatments

Monocytes were directly cultured with wild-type or TNF, TNFR1, TNFR2 KO NB cell lines in ULA plates for 24 hours. Activation of NF-kB in monocytes and NB was measured by flow cytometry and cytokine production measured by ELISA from co-culture supernatant. To evaluate the contact-dependence of monocyte activation by NB, monocytes were cultured in ULA plates with seven-day NB conditioned medium mixed 1:1 with complete monocyte medium for 24 hours and analyzed for IL-6 production. To assess the growth advantage of NB cultured with monocytes, NB-luc cell lines and monocytes were seeded in tissue culture-treated white 96-well plates (1:1 ratio) and cultured for four days. Luminescence was measured using a TECAN Spark plate reader (TECAN). To evaluate changes in proliferation, NB was pulsed for 10 minutes in CellTrace Violet (2 μM, Invitrogen) and seeded at 100,000 cells per well in a 12-well tissue culture-treated plate. Monocytes were added directly to the NB monolayer at a density of 100,000 per well and incubated for four days. CellTrace Violet expression was analyzed by flow cytometry.

Where indicated, co-cultures of NB and monocytes were pre-treated for 15 minutes with TNF pathway neutralizing antibodies at 10 μg/mL: anti-TNFα (R&D Systems, Clone #1825), anti-TNFR1 (R&D Systems, Clone #16803), anti-TNFR2 (R&D Systems, Clone #22210), or mouse IgG1 control (R&D Systems, Clone #11711). Etanercept (Amgen) and human IgG control (MP Biomedical) were used at 10 μg/mL. Small molecule inhibitors of TNFα or NF-kB signaling were used at indicated concentrations: TAPI-1 (40 μm, Sigma) and IKK Inhibitor VII (0-20 μM, APExBio).

CRISPR

CRISPR/Cas9-mediated KO of TNFα (TNF), TNFR1 (TNFRSF1A), and TNFR2 (TNFRSF1B) was performed as previously described (48). Briefly, single guide RNAs (sgRNAs) targeting the first or second coding exons of TNF, TNFRSF1A, and TNFRSF1B were designed using CRISPRscan and selected for low predicted off-target effects and low off-target cutting frequency determination (CFD) score (49, 50). Oligos used for sgRNA synthesis were ordered from Sigma (SEQ ID NOs: 13-26). sgRNA guides were in vitro transcribed using the HiFi T7 Transcription Kit (NEB) from sgDNA intermediates that were generated by PCR from guide-specific oligo forward primers, a universal reverse primer, and the px458 plasmid DNA template. sgRNA in vitro reactions were concentrated using RNA Clean & Concentrate 25 (Zymo). Cas9 (PNABio) and sgRNA guides were mixed (2 μg Cas9, 1 μg each guide) and incubated for 20 minutes at room temperature to form ribonucleoprotein (RNP) complexes. RNPs were electroporated into NB cell lines using the Neon Electroporation System (ThermoFisher) with optimized protocols (CHLA-255 1600V, 10ms, 3 pulses; SK-N-AS & SK-N-BE(2) 1450V, 20 ms, 2 pulses). Cells were seeded at a density of 0.5 cells per well in 96-well tissue culture plates and grown until colonies were visible. Plates of single-cell clones were imaged using the Incucyte s3 Live Cell Image system (Essenbio/Sartorius) and wells with clear single clones were expanded and genotyped for genomic deletion. Homozygous knockouts were sequenced (Genewiz) and functional knockout confirmed by loss of protein expression via flow cytometry or lysate ELISA.

Flow Cytometry

To evaluate the expression of TNFα in NB and monocytes, cells were first stimulated with LPS (50 ng/mL) in the presence of GolgiStop (BD Biosciences) and fixed using CytoFix/Cytoperm buffer (BD Biosciences). Cells were then stained with TNFα-PE (BD Biosciences). To evaluate the expression of TNFR1 and TNFR2 on NB and monocytes, cells were stained with TNFR1-BV421 and TNFR2-APC (BD Biosciences) in FACS staining buffer containing 0.05% sodium azide to prevent receptor/antibody processing. To estimate changes in NF-kB expression, treated co-cultures were first stained with ZombieViolet (1 μL/test, Biolegend) to counterstain dead cells and APC-H7 CD45 and/or FITC-CD14 to label monocytes for 20 minutes at room temperature. Cells were fixed with Phosflow Fix Buffer I (BD Biosciences) for 15 minutes at 37 C, followed by ice-cold 1× Phosflow Perm Buffer IV (BD Biosciences) for 20 minutes at 4 C. Fixed and permeabilized cells were then stained with PE-IκBα (Total) (BD Biosciences) for 30 minutes at room temperature. To evaluate changes in proliferation, CellTrace Violet-pulsed NB co-cultured with monocytes was collected and first counterstained with APC-H7-CD14 (BD Biosciences) for 30 minutes at 4 C. Stained samples were resuspended in FACS staining buffer with 7-AAD (10 μL/test, BD Biosciences) to counterstain dead cells. All flow samples were run using an LSRII 5-laser flow cytometer (Texas Children's Hospital Cancer Center Flow Cytometry Core, Houston, Tex.) and analyzed using FlowJo Software (Treestar).

Western Blot

Wild-type and TNFR1/2 KO SK-N-BE(2) cells were lysed in RIPA buffer (ThermoFisher) supplemented with protease inhibitor cocktail (Roche) for 20 minutes on ice then centrifuged for 20 min at 14,000 rpm. Cleared lysate was transferred to a new tube and concentration determined using the Bio-Rad protein assay (Bio-Rad). Samples were diluted to 50 μg/40 μL in 4× sample loading buffer (LI-COR) with 2-mercaptoethanol and RIPA buffer. Samples were run on 12% TGX gels (Bio-Rad) at 100V for 60-90 minutes followed by wet transfer onto 0.45 μm PVDF membranes in 1× tris-glycine (ThermoFisher) supplemented with 20% methanol at 100V for 60 minutes. Following transfer, membranes were blocked in 0.5× Odyssey PBS blocking buffer (LI-COR) for one hour at room temperature. Primary antibodies were diluted at the following concentrations in 0.5× Odyssey blocking buffer with 0.1% Tween20 (ThermoFisher) and incubated overnight at 4° C.: anti-GAPDH (Mouse mAb#5174, CST, 1:1000), anti-GAP43 (Rabbit mAb#8945, CST, 1:500), and anti-enolase-2 (Rabbit mAb #24330, CST, 1:500). The following secondary antibodies were diluted 1:5000 in 0.5× Odyssey blocking buffer with 0.1% Tween20 (ThermoFisher) and incubated one hour at RT: IRDye-680 Goat anti-Rabbit IgG and IRDye-800 Goat anti-Mouse (LI-COR). Membranes were imaged using the near-IR imaging system, Odyssey CLx Imager (LI-COR).

Cytokine Analysis

Cytokines released by monocytes were detected within co-culture supernatants using the Human TNF-alpha Quantikine ELISA Kit (TNFα, R&D Systems) and the Human IL-6 Quantikine ELISA Kit (IL-6, R&D Systems) according to manufacturer's guidelines. ELISA plates were read using a TECAN Spark plate reader (TECAN). Global cytokine regulation was evaluated using a custom Luminex panel containing the following cytokines: CX3CL1, CXCL10, EGF, FGF-2, Flt3L, G-CSF, GM-CSF, IL-1α, IL-1β, IL-1Ra, IL-4, IL-6, IL-8, IL-10, IL-12 (p40), IL-12 (p′70), M-CSF, MCP-1, MCP-3, MDC, MIP-1α, MIP-1β, TGFα, TNFα, TNFβ, and VEGF-A (Human Cytokine Panel A, Millipore-Sigma).

RNA Analysis

Total RNA from cell pellets was isolated using the RNeasy Micro Plus Kit (Qiagen). One-step cDNA synthesis and qRT-PCR was performed using the KAPA SYBR Fast One Step qRT-PCR kit (Roche) with the CFX96 Touch RT-PCR Detection System (BioRad). Primers were from Sigma (SEQ ID NOs: 13-26). Relative changes in gene expression were calculated based on the ΔCt method using housekeeping gene GAPDH as control. Snap-frozen tumor specimens were divided into 30 mg fractions and incubated in RLT buffer (Qiagen). Tumors were homogenized using manual microtube homogenizers (BioMasher, Takara) and the lysates were then sheared using QiaShredder columns (Qiagen). Total RNA was isolated using the RNeasy Micro Plus Kit with gDNA elimination columns (Qiagen).

Eukaryotic mRNA sequencing with 30M depth and 150 paired end reads was performed by Novogene, Inc. Quality control checks on FASTQ sequencing files were performed by FastQC (v0.11.2, https://www.bioinformatics.babraham.ac.uk/projects/fastqc/). Reads were then aligned to human (GRCh38, Ensembl release version 84) or mouse (GRCm38, Ensembl release version 81) reference genomes separately by hisat2 v2.1.0(51). Using the XenoFilteR workflow (52), the mouse genome was aligned to the human transcripts to remove reads that mapped to both genomes, resulting in reads unique to human (representing NB tumor cells). The opposite process was performed using the human genome on the mouse transcript file to identify reads unique to mouse (tumor microenvironment). The filtered reads were then subject to transcript assembly and quantification using stringtie v1.3.5 (53). The unique human and mouse transcripts were then subjected to differential gene expression analysis (DESeq2 R package based on raw counts) using a two-fold change difference in expression, and p-adj.<0.05 as cutoff thresholds.

In Vivo Experiments

Six-week old female NOD/SCID/IL-2Ry-null (NSG) mice were purchased from The Jackson Laboratory and maintained at the Baylor College of Medicine (BCM) animal care facility. At eight-weeks of age, mice were injected in the subcutaneous right flank with either 1×106 CHLA-255-luc (or SK-N-AS-luc) or a combination of 1×106 CHLA-255-luc and 1×106 human monocytes (or SK-N-AS-luc/monocyte) embedded in growth factor-reduced Matrigel (Corning) as previously described (54). Where indicated, mice were injected i.p. with 100 μg/mouse of etanercept (Amgen), or isotype human IgG control (MP Biomedical) twice a week for the entire three-week experiment duration. Tumor growth was measured indirectly by weekly bioluminescent imaging (Small Animal Imaging Core Facility, Texas Children's Hospital). After three weeks, mice were euthanized and tumors were saved for IHC (see Tumor IHC) and RNA-seq (see RNA Analysis).

Tumor IHC

Tumors from in vivo experiments were snap-frozen in OTC Media (Tissue-Tek, VWR) and cryo-sectioned into 5 μm thin sections (Texas Children's Hospital Histology Core, Houston, Tex.). Samples were fixed and permeabilized in ice-cold acetone for 10 minutes at −20 C and blocked in TBS containing 5% normal goat serum (CST) and 1% BSA (Sigma) for 2 hours at RT. Rat anti-mouse CD31 primary antibody (Invitrogen, clone 390) was diluted 1:10 in staining buffer (TBS, 1% NGS, 1% BSA) overnight at 4 C. Goat anti-rat Alexa Fluor 594 secondary antibody (Invitrogen) was diluted 1:500 in staining buffer and incubated on slides for one hour, light-shielded, at room temperature. Slides were counterstained with 1 μg/mL DAPI (BD Biosciences) for five minutes and mounted with fluorescence mounting media (Fluoromount G, Invitrogen). Tumors were imaged using a DeltaVision Live High-Resolution Deconvolution microscope (GE Healthcare) in an unbiased manner at the BCM Integrated microscopy core (Houston, Tex.). Images were processed and analyzed using FIJI/ImageJ software (NIH). To quantify CD31+ staining, images were thresholded (default, 1800 MIN, Infinity MAX), converted to binary, and analyzed using the FIJI/ImageJ “Analyze Particle” function excluding regions smaller than 25 μm2. These analysis parameters were used across all samples and FOVs.

Computational Data

Analysis of human NB samples was performed using a pre-existing database within R2: Genomics Analysis and Visualization Platform (http://r2.amc.nl). “Tumor Neuroblastoma—Kocak—649” was used to analyze differential gene expression pathways between stage IV MYCN-A and -NA tumors. “Tumor Neuroblastoma non MYCN amplified—Seeger—102” was used for Kaplan Meier analysis of TNF (TNFα), TNFRSF1A (TNFR1) and TNFRSF1B (TNFR2) gene expression and to correlate pathway gene expression with survival in MYCN-NA disease. To evaluate TNF signaling and progression, the dataset was first subjected to differential gene expression analysis in patients that progressed versus those that did not progress using p<0.05 and two-fold expression cutoffs. Gene set enrichment analysis (over-representation test) within the differentially regulated genes was performed using 2×2 contingency table analysis with continuity correction. Statistical tests, as indicated, were performed within R2 and p<0.05 was considered significant.

Statistics

Statistical analysis was performed using GraphPad Prism 8.0 software (GraphPad). Unless otherwise indicated, the distribution of data was assumed normal, and parametric analyses were performed. Comparisons between groups were performed using one-or two-way ANOVA with recommended post-test correction for multiple comparisons. All statistical tests were two-sided and a P value less than 0.05 was considered statistically significant.

Study Approval

Animal experiments were performed according to IACUC-approved protocol AN-5194 at Baylor College of Medicine.

PCR Primers

| Primer | Use | Sequence (5′-->3′) | SEQ ID |
|---|---|---|---|
| TNFα | PCR | AACCGAGACAGAAGGTGCAG | SEQ ID |
| Forward | | | NO: 13 |
| TNFα | PCR | TCTGTGTGCCAGACACCCTA | SEQ ID |
| Reverse | | | NO: 14 |
| TNFR1 | PCR | GAAGAAGGGGCCATCACTGAA | SEQ ID |
| Forward | | | NO: 15 |
| TNFR1 | PCR | TGCAAGTGAGTGACAAGAACAG | SEQ ID |
| Reverse | | | NO: 16 |
| TNFR2 | PCR | GGTGGGTCCCATGTGAGAGA | SEQ ID |
| Forward | | | NO: 17 |
| TNFR2 | PCR | ACGAGGGCAAGGGTTTTGTC | SEQ ID |
| Reverse | | | NO: 18 |
| TNFα | RT-qPCR | GAAAGCATGATCCGGGACGTG | SEQ ID |
| Forward | | | NO: 19 |
| TNFα | RT-qPCR | GATGGCAGAGAGGAGGTTGAC | SEQ ID |
| Reverse | | | NO: 20 |
| TNFR1 | RT-qPCR | TCACCGCTTCAGAAAACCACC | SEQ ID |
| Forward | | | NO: 21 |
| TNFR1 | RT-qPCR | GGTCCACTGTGCAAGAAGAGA | SEQ ID |
| Reverse | | | NO: 22 |
| TNFR2 | RT-qPCR | TTCATCCACGGATATTTGCAGG | SEQ ID |
| Forward | | | NO: 23 |
| TNFR2 | RT-qPCR | GCTGGGGTAAGTGTACTGCC | SEQ ID |
| Reverse | | | NO: 24 |
| GAPDH | RT-qPCR | GAAGGTGAAGGTCGGAGTC | SEQ ID |
| Forward | | | NO: 25 |
| GAPDH | RT-qPCR | GAAGATGGTGATGGGATTTC | SEQ ID |
| Reverse | | | NO: 26 |

### XVIII. Example 18: Tumor-Cell TNFR2 and Monocyte Membrane TNFα

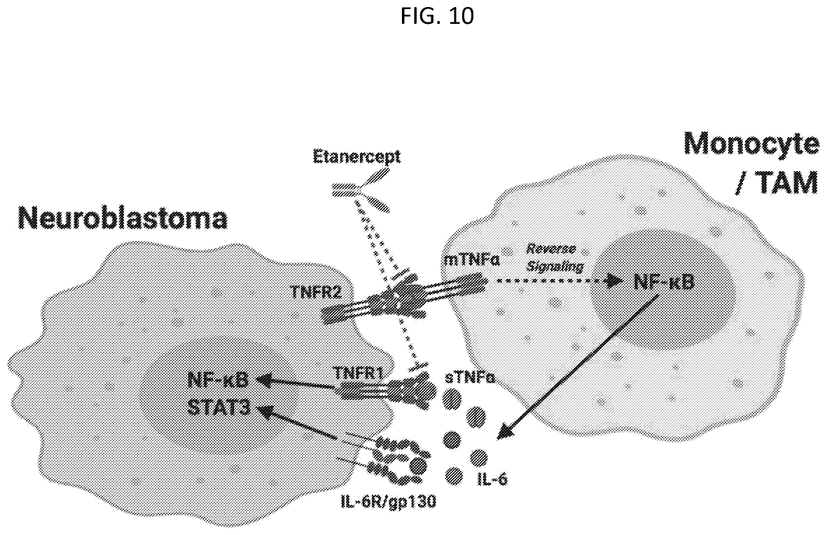

Certain embodiments described herein define a novel positive feedback loop by which NB cells activate monocytes, initiating a self-sustaining inflammatory reaction that supports tumor growth (FIG. 10). Central to this mechanism, certain embodiments demonstrate there is an unexpectedly fundamental role for both secreted and membrane-bound TNFα signaling in the crosstalk between NB and monocytic cells. In detail, NB cell TNFR2 reverse signals through mTNFα expressed on monocytes, causing monocytes to produce sTNFα that in turn binds to TNFR1 on NB cells and activates pro-survival NF-κB signaling. In addition to sTNFα, mTNFα-activated monocytes produce cytokines including IL-6 and G-CSF, which have been shown to promote NB growth via STAT3 activation. Consistent with the role of the novel TNFR2-TNFα-TNFR1 axis in the described mechanism, it was found that expression of TNFα receptors and other genes associated with the TNFα pathway are highly predictive of poor outcome in NB patients. Importantly, targeting TNFα with FDA-approved etanercept blocks monocyte activation by NB cells in vitro and inhibits angiogenesis and tumor growth in vivo, showing promise for potential clinical applications, which are included in some embodiments encompassed herein.

It had been previously reported that activation of NF-κB and CCL20 production by monocytes co-cultured with NB cells is a contact-dependent process that requires TNFα (24), suggesting that NB and monocytes interact via mTNFα. Here, evidence is provided that cell-cell contact between NB cells and monocytes is indeed required for activation of NF-κB in monocytes and production of downstream cytokines, and that this cascade begins with interaction of mTNFα on monocytes and TNFR2 on NB cells. Elucidation of this mechanism provides critical insight into how NB cells initiate tumor-supportive inflammation. Interestingly, while both NB cells (24, 25) and monocytes (26, 27) have been reported to express mTNFα, none of the eight NB cell lines that were tested expressed levels of TNF mRNA that could be detected by qRT-PCR. To follow up on this inconsistency, a commonly used anti-mTNFα monoclonal antibody (clone 6401) was tested in validated TNFα KO NB cells; positive staining observed in these cells demonstrated the non-specific activity of this antibody (data not shown). Further, TNFα KO NB clones activated monocytes to the same extent as wild-type NB, suggesting that monocytes, and not NB cells, are the relevant source of mTNFα in the NB-monocyte axis. The results of the experiments encompassed in embodiments herein in which monocytes were treated with a TACE inhibitor, which blocks TNFα secretion and stabilizes mTNFα expression, further support the critical role of monocytic mTNFα in activation of monocytes by NB cells. Consistent with the observation of mTNFα-mediated reverse signaling in monocytes, the mTNFα isoform has been reported to function as both a ligand (forward signaling) and a receptor (reverse signaling) through a poorly understood mechanism, while in macrophages and myeloid cells it is thought to depend on TGFβ signaling (28). In tumor cells, mTNFα reverse signaling may directly result in constitutive NF-κB signaling (29, 30). While NB as a source of TNFα for monocyte activation was excluded, there remains the possibility that NB cells produce an undetectable amount of TNFα that is sufficient to stimulate growth in an autocrine fashion; such an effect could explain growth inhibition and induction of differentiation observed in TNFR1 and TNFR2 KO clones of several NB cell lines. Alternatively, this phenomenon could be explained by self-activation of NB TNFα receptors, which would require further investigation.

The essential role of TNFα in the NB-monocyte interaction is further evidenced by experiments with etanercept, an Fc-TNFR2 fusion protein that mimics and therefore competes with NB cell TNFR2 for binding to monocytic mTNFα. Strikingly, it was found that etanercept blocks physical aggregation of NB cells and monocytes, abrogates the pro-tumorigenic axis between monocytes and NB cells, and shuts down production of the full network of inflammatory cytokines. The prominent loss of tumorsphere formation following treatment with etanercept further supports the notion that TNFR2 initiates interaction between NB cells and monocytes. Several cytokines induced by this interaction—including IL-6, G-CSF, sTNFα, and IL-1βP—have been shown individually to promote tumor growth in numerous pre-clinical NB models and clinical studies (31-36).

In particular, the role of IL-6 in supporting NB growth, metastasis, and resistance to therapy has been investigated extensively (9, 31, 32). For example, it was previously demonstrated that tumor-supportive TAMs are a major source of IL-6 in primary NB tumors (9), and others have shown that IL-6 promotes NB cell proliferation and resistance to apoptosis in a manner that depends on STAT3 and ERK1/2 signaling (31, 32). Clinically, elevated levels of IL-6 in the serum and bone marrow of NB patients have been associated with lower event-free survival (37), and expression of IL-6R in MYCN-non-amplified NB tumors has been shown to correlate inversely with event-free survival (10). However, in an IL-6 KO transgenic NB model, absence of IL-6 did not impact tumor development and prevented neither STAT3 activation nor MYC upregulation (38), suggesting that IL-6 plays a redundant role in NB progression. Indeed, other STAT3-activating cytokines have been shown to contribute to NB growth; for example, G-CSF drives tumor growth and metastasis in mouse xenograft NB models by supporting NB cells with cancer stem cell properties (34, 35). Another recent study showed that NB cells, including primary tumor cells from patients, induce monocytes to produce sTNFα and IL-1β (36). These cytokines in turn induce NB cells to express ARG2, which catabolizes arginine and generates metabolic changes that favor NB growth and contribute to the immunosuppressive TME. The understanding that interaction of NB TNFR2 with monocytic mTNFα triggers an inflammatory reaction that results in production of multiple tumor-promoting cytokines, and that this reaction can be terminated by treatment with etanercept illuminates a promising therapeutic opportunity for high-risk NB patients.

The proof-of-concept therapeutic experiments demonstrate that etanercept also impacts tumor growth in vivo—NSG mice engrafted with human NB cells and primary human monocytes had smaller, less vascularized tumors when they were treated with etanercept compared to tumors from control mice. This disruption of the TME is consistent with the role of TAMs and the cytokines they produce in tumor angiogenesis, invasion, and metastasis in many types of cancer (39, 40). Gene expression analysis of the NB xenografts revealed that etanercept-treated tumors had decreased ability to respond to stress and hypoxia, as well as decreased expression of genes associated with the pro-survival MAPK pathway that is activated by TNFα, IL-6, IL-1β, and other cytokines released during the innate immune response (41). The RAS-MAPK pathway is also commonly impacted by gain-of-function mutations in many types of cancer (42); indeed, mutations in this pathway were recently shown to occur frequently in relapsed NB and to correlate with poor survival (43). This suggests that tumor-induced inflammation not only creates a TME that is conducive to tumor progression and activates pro-survival NF-κB and STATS signaling pathways, but can also enhance key cell-intrinsic oncogenic signaling in NB cells. Therefore, combining etanercept with small-molecule MEK inhibitors could be a promising therapeutic strategy for recurrent/resistant NB. It is also likely that by disrupting the immunosuppressive TME, etanercept could be effectively combined with immunotherapies including the current standard-of-care for high-risk NB, anti-GD2 monoclonal antibody dinutuximab (44), or with adoptive cell therapies such as chimeric antigen receptor (CAR)-T or CAR-NKT cells that are currently in early-stage clinical trials (45, 46). Overall, elucidation of this novel molecular feedback loop that underlies a tumor-initiated inflammation cascade will inform the design of new pathophysiology-driven therapeutic strategies for high-risk NB and other types of cancer.

### XIX. Example 19: Interaction Triggering Tumorigenic Inflammation

Tumor progression and resistance to therapy in children with neuroblastoma (NB), a common childhood cancer, are often associated with infiltration of macrophages that produce inflammatory cytokines. Over the last decade, several groups have demonstrated the role of individual pro-inflammatory cytokines including IL-6, G-CSF, soluble (s)TNFα, and IL-1β in supporting tumor growth and metastasis in murine NB models and in correlative clinical studies. However, the mechanism by which NB tumor-supportive inflammation is initiated and propagated has remained unknown, precluding rational therapeutic interventions. Certain embodiments herein demonstrate a previously unanticipated central role for TNFα in the crosstalk between NB and monocytic cells. Specifically, a novel positive feedback loop has been identified where NB cell TNFR2 reverse signals through membrane (m)TNFα expressed on monocytes, causing them to produce sTNFα that binds in turn to TNFR1 on NB cells and activates pro-survival NF-κB signaling. In addition to sTNFα, mTNFα-activated monocytes produce multiple cytokines including IL-6, G-CSF, and IL-1β. The results explain how these distinct effector cytokines work in concert to drive tumor progression. Importantly, it was found that targeting both mTNFα and sTNFα with FDA-approved etanercept blocks activation of monocytes by NB cells, shutting down the entire network of tumorigenic cytokines and inhibiting tumor growth in vivo.

Embodiments herein may represent a major conceptual advance in the field of cancer biology, revealing in intricate detail the mechanism by which tumor-supportive inflammation is initiated and propagated, as well as how it can be targeted for therapy in patients with high-risk NB. Considering the importance of the inflammatory tumor-microenvironment in tumor initiation, progression, and resistance to therapy in many types of cancer, the methods, compositions, systems, and embodiments encompassed herein are useful in many types of cancer.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method for treating neuroblastoma in an individual, comprising administering a therapeutically effective amount of one or more tumor necrosis factor (TNF) inhibitors to the individual.

2. The method of claim 1, wherein the TNF inhibitor comprises at least one small molecule, immunotherapy, cell therapy, peptide, peptide derivative, antibody, fusion protein, glycoprotein, nucleic acid, nucleic acid derivative, or a combination thereof.

3. The method of claim 1, wherein the TNF inhibitor comprises a TNFα inhibitor, a TNFR1 inhibitor, a TNFR2 inhibitor, or a combination thereof.

4. The method of claim 3, wherein the TNFα inhibitor comprises an inhibitor of soluble TNFα and/or membrane-bound TNFα.

5. The method of claim 1, wherein the TNF inhibitor comprises etanercept, infliximab, certolizumab, golimumab, adalimumab, thalidomide, lenalidomide, pomalidomide, a xanthine derivative, bupropion, 5-HT2A agonist hallucinogen, or a combination thereof.

6. The method of claim 1, wherein the TNF inhibitor comprises etanercept.

7. The method of claim 1, wherein the administration is intravenously, intradermally, transdermally, intrathecally, intraarterially, intraperitoneally, intranasally, intravaginally, intrarectally, topically, intramuscularly, subcutaneously, mucosally, orally, topically, locally, by inhalation, by injection, by infusion, by continuous infusion, via a catheter, or via a lavage.

8. The method of claim 6, wherein the etanercept is administered subcutaneously.

9. The method of claim 1, wherein the one or more TNF inhibitors is administered, either concurrently or sequentially, with a therapy for treating neuroblastoma.

10. The method of claim 1, wherein the individual is a human with neuroblastoma.

11. The method of claim 10, wherein the individual is a pediatric patient with neuroblastoma.

12. A method of reducing tumor-promoting inflammation in an individual, comprising contacting cells and/or extracellular fluid from the individual with a therapeutically effective amount of one or more TNF inhibitors.

13. The method of claim 12, wherein the TNF inhibitor comprises at least one small molecule, immunotherapy, cell therapy, peptide, peptide derivative, antibody, fusion protein, glycoprotein, nucleic acid, nucleic acid derivative, or a combination thereof.

14. The method of claim 12, wherein the TNF inhibitor comprises a TNFα inhibitor, a TNFR1 inhibitor, a TNFR2 inhibitor, or a combination thereof.

15. The method of claim 14, wherein the TNFα inhibitor comprises an inhibitor of soluble TNFα and/or membrane-bound TNFα.

16. The method of claim 12, wherein the TNF inhibitor comprises etanercept, infliximab, certolizumab, golimumab, adalimumab, thalidomide, lenalidomide, pomalidomide, a xanthine derivative, bupropion, 5-HT2A agonist hallucinogen, or a combination thereof.

17. The method of claim 12, wherein the TNF inhibitor comprises etanercept.

18. The method of claim 12, wherein reducing the tumor-promoting inflammation comprises reducing IL-6, soluble TNFα, and/or membrane-bound TNFα, and/or blocking NFκB and/or Stat3 signaling.

19. The method of claim 12, wherein the contacting occurs in vitro, in vivo, and/or ex vivo.

20. The method of claim 12, wherein the cells are capable of producing IL-6 and/or TNFα, and/or express a TNF and/or IL-6 receptor.

21. The method of claim 12, wherein the cells are cancerous.

22. The method of claim 12, wherein the cells comprise monocytes and/or macrophages

23. The method of claim 12, wherein the cells are neuroblastoma cells.

24. The method of claim 12, wherein the extracellular fluid comprises blood, plasma, serum, interstitial fluid, tumor interstitial fluid, cell culture fluid, or a combination thereof
